(19) United States
(12) Patent Application Publication
(10) Pub. No.: US 2013/0281890 A1
(43) Pub. Date: Oct. 24, 2013

(54) NEUROMODULATION DEVICES AND METHODS

(71) Applicant: David J. MISHELEVICH, Playa del Rey, CA (US)

(72) Inventor: David J. MISHELEVICH, Playa del Rey, CA (US)

(21) Appl. No.: 13/918,862

(22) Filed: Jun. 14, 2013

Related U.S. Application Data
(63) Continuation-in-part of application No. 12/958,411, filed on Dec. 2, 2010.
Application No. 12/958,411 is a continuation-in-part of application No. 12/940,052, filed on Nov. 5, 2010.
Application No. 12/940,052 is a continuation-in-part of application No. 13/007,626, filed on Jan. 15, 2011.
Application No. 13/007,626 is a continuation-in-part of application No. 13/200,903, filed on Jan. 15, 2011.
Application No. 13/200,903 is a continuation-in-part of application No. 13/694,327, filed on Jan. 16, 2011.
Application No. 13/694,327 is a continuation-in-part of application No. 13/694,328, filed on Jan. 16, 2011.
Application No. 13/694,328 is a continuation-in-part of application No. 13/098,473, filed on May 1, 2011.
Application No. 13/098,473 is a continuation-in-part of application No. 13/360,600, filed on Jan. 27, 2012.
Application No. 13/360,600 is a continuation-in-part of application No. 13/252,054, filed on Oct. 3, 2011.
Application No. 13/252,054 is a continuation-in-part of application No. 13/625,677, filed on Sep. 24, 2012.
Application No. 13/625,677 is a continuation-in-part of application No. 13/689,178, filed on Nov. 29, 2012.
Application No. 13/689,178 is a continuation-in-part of application No. 13/718,245, filed on Dec. 18, 2012.
Application No. 13/718,245 is a continuation-in-part of application No. 13/689,178, filed on Nov. 29, 2012.
(60) Provisional application No. 61/266,112, filed on Dec. 2, 2009.
(60) Provisional application No. 61/260,172, filed on Nov. 11, 2009.
(60) Provisional application No. 61/295,757, filed on Jan. 17, 2010.
(60) Provisional application No. 61/295,760, filed on Jan. 18, 2010.
(60) Provisional application No. 61/295,759, filed on Jan. 18, 2010.
(60) Provisional application No. 61/295,761, filed on Jan. 18, 2010.
(60) Provisional application No. 61/325,339, filed on Apr. 18, 2010.
(60) Provisional application No. 61/330,363, filed on May 2, 2010.
(60) Provisional application No. 61/436,607, filed on Jan. 27, 2011.
(60) Provisional application No. 61/389,280, filed on Oct. 4, 2010.
(60) Provisional application No. 61/538,934, filed on Sep. 25, 2011.
(60) Provisional application No. 61/564,856, filed on Nov. 29, 2011.
(60) Provisional application No. 61/564,856, filed on Nov. 29, 2011.
(60) Provisional application No. 61/577,095, filed on Dec. 19, 2011.
(60) Provisional application No. 61/666,825, filed on Jun. 30, 2012.

(51) Int. Cl. A61N 7/00

(52) U.S. Cl. CPC A61N 7/00
USPC 601/2; 607/3

(57) ABSTRACT

Disclosed are methods and systems for deep or superficial deep-brain stimulation using multiple therapeutic modalities, including up-regulation or down-regulation using ultrasound impacting one or multiple points in a neural circuit to produce Long-Term Potentiation (LTP) or Long-Term Depression (LTD). Also disclosed are: methods and systems for patient-feedback control of non-invasive deep brain or superficial neuromodulation; devices for producing shaped or steered ultrasound for non-invasive deep brain or superficial neuromodulation; methods and systems using intersecting ultrasound beams; non-invasive ultrasound-neuromodulation techniques to control the permeability of the blood-brain barrier; non-invasive neuromodulation of the spinal cord by ultrasound energy; methods and systems for non-invasive neuromodulation using ultrasound for evaluating the feasibility of neuromodulation treatment using non-ultrasound/ultrasound modalities; and method and systems for neuromodulation using ultrasound delivered in sessions.

### CROSS REFERENCE TO RELATED APPLICATION

This application is a continuation-in-part of U.S. patent application Ser. No. 12/958,411, filed Dec. 2, 2010, titled “MULTI-MODALITY NEUROMODULATION OF BRAIN TARGETS,” Publication No. US 2011-0130615 A1, which claims priority to U.S. Provisional Patent Application No. 61/266,112, filed Dec. 2, 2009, and titled entitled “MULTI-MODALITY NEUROMODULATION OF BRAIN TARGETS,” each of which is herein incorporated by reference in its entirety.

This application is a continuation-in-part of U.S. patent application Ser. No. 12/940,052, filed Nov. 5, 2010, titled “NEUROMODULATION OF DEEP-BRAIN TARGETS USING FOCUSED ULTRASOUND,” Publication No. US 2011-0112394 A1, which claims priority to U.S. Provisional Patent Application No. 61/260,172, filed Nov. 11, 2009, and titled “STIMULATION OF DEEP BRAIN TARGETS USING FOCUSED ULTRASOUND FILED,” and U.S. Provisional Patent Application No. 61/295,757 filed Jan. 17, 2010, and titled “NEUROMODULATION OF DEEP BRAIN TARGETS USING FOCUSED ULTRASOUND,” each of which is herein incorporated by reference in its entirety.

This application is a continuation-in-part of U.S. patent application Ser. No. 13/007,626, filed Jan. 15, 2011, titled “PATIENT FEEDBACK FOR CONTROL OF ULTRASOUND DEEP-BRAIN NEUROMODULATION,” Publication No. US 2011-0178442 A1, which claims priority to U.S. Provisional Patent Application No. 61/295,760, filed Jan. 18, 2010, and titled “PATIENT FEEDBACK FOR CONTROL OF ULTRASOUND FOR DEEP-BRAN NEUROMODULATION,” each of which is herein incorporated by reference in its entirety.

This application is a continuation-in-part of U.S. patent application Ser. No. 13/200,903, filed Jan. 15, 2011, titled “SHAPED AND STEERED ULTRASOUND FOR DEEP-BRAIN NEUROMODULATION,” Publication No. US 2012-0053391 A1, which claims priority to U.S. Provisional Patent Application No. 61/295,759, filed Jan. 18, 2010, and titled “SHAPED AND STEERED ULTRASOUND FOR DEEP-BRAIN NEUROMODULATION,” each of which is herein incorporated by reference in its entirety.

This application is a continuation-in-part of U.S. patent application Ser. No. 13/694,327, filed Jan. 16, 2011, titled “TREATMENT PLANNING FOR DEEP-BRAIN NEUROMODULATION,” Publication No. US 2013-0066350 A1, which claims priority to U.S. Provisional Patent Application No. 61/295,761, filed Jan. 18, 2010, and titled “TREATMENT PLANNING FOR DEEP-BRAIN NEUROMODULATION,” each of which is herein incorporated by reference in its entirety.

This application is a continuation-in-part of U.S. patent application Ser. No. 13/694,328, filed Jan. 16, 2011, titled “ULTRASOUND NEUROMODULATION OF THE BRAIN, NERVE ROOTS, AND PERIPHERAL NERVES,” Publication No. US 2013-0066239 A1, which claims priority to U.S. Provisional Patent Application No. 61/325,339, filed Apr. 18, 2010, and titled “ULTRASOUND NEUROMODULATION OF THE BRAIN, NERVE ROOTS, AND PERIPHERAL NERVES,” each of which is herein incorporated by reference in its entirety.

This application is a continuation-in-part of U.S. patent application Ser. No. 13/098,473, filed May 1, 2011, titled “ULTRASOUND MACRO-PULSE AND MICRO-PULSE SHAPES FOR NEUROMODULATION,” Publication No. US 2011-0270138 A1, which claims priority to U.S. Provisional Patent Application No. 61/330,363, filed May 2, 2010, and titled “ULTRASOUND MACRO-PULSE AND MICRO-PULSE SHAPES FOR NEUROMODULATION,” each of which is herein incorporated by reference in its entirety.

This application is a continuation-in-part of U.S. patent application Ser. No. 13/360,600, filed Jan. 27, 2012, titled “PATTERNED CONTROL OF ULTRASOUND FOR NEUROMODULATION,” Publication No. US 2012-0197163 A1, which claims priority to U.S. Provisional Patent Application No. 61/436,607, filed Jan. 27, 2011, and titled “PATTERNED CONTROL OF ULTRASOUND FOR NEUROMODULATION,” each of which is herein incorporated by reference in its entirety.

This application is a continuation-in-part of U.S. patent application Ser. No. 13/252,054, filed Oct. 3, 2011, titled “ULTRASOUND-INTERSECTING BEAMS FOR DEEP-BRAIN NEUROMODULATION,” Publication No. US 2012-0083719 A1, which claims priority to U.S. Provisional Patent Application No. 61/389,280, filed Oct. 4, 2010, and titled “ULTRASOUND-INTERSECTING BEAMS FOR DEEP-BRAIN NEUROMODULATION,” each of which is herein incorporated by reference in its entirety.

This application is a continuation-in-part of U.S. patent application Ser. No. 13/625,677, filed Sep. 24, 2012, titled “ULTRASOUND-NEUROMODULATION TECHNIQUES FOR CONTROL OF PERMEABILITY OF THE BLOOD-BRAIN BARRIERUS,” Publication No. US 2013-0079682 A1, which claims priority to U.S. Provisional Patent Application No. 61/538,934, filed Sep. 25, 2011, and titled ULTRASOUND-NEUROMODULATION TECHNIQUES FOR CONTROL OF PERMEABILITY OF THE BLOOD-BRAIN BARRIER,” each of which is herein incorporated by reference in its entirety.

This application is a continuation-in-part of U.S. patent application Ser. No. 13/689,178, filed Nov. 29, 2012, titled “ULTRASOUND NEUROMODULATION OF SPINAL CORD,” which claims priority to U.S. Provisional Patent Application No. 61/564,856, filed Nov. 29, 2011, and titled “ULTRASOUND NEUROMODULATION OF THE SPINAL CORD,” each of which is herein incorporated by reference in its entirety.

This application is a continuation-in-part of U.S. patent application Ser. No. 13/718,245, filed Dec. 18, 2012, titled “ULTRASOUND NEUROMODULATION FOR DIAGNOSIS AND OTHER-MODALITY PREPLANNING,” which is a continuation-in-part of U.S. patent application Ser. No. 13/689,178, filed Nov. 29, 2012, titled “ULTRASOUND NEUROMODULATION OF SPINAL CORD,” which claims priority to U.S. Provisional Application No. 61/564,856, filed Nov. 29, 2011, titled “ULTRASOUND NEUROMODULATION OF SPINAL CORD.” U.S. patent application Ser. No. 13/718,245 also claims priority to U.S. Provisional Patent Application No. 61/577,095, filed Dec. 19, 2011 and titled “ULTRASOUND NEUROMODULATION FOR DIAGNOSIS AND OTHER-MODALITY PREPLANNING,” each of which is herein incorporated by reference in its entirety

This application claims priority to U.S. Provisional Patent Application No. 61/666,825, filed Jun. 30, 2012, titled “ULTRASOUND NEUROMODULATION DELIVERED IN SESSIONS,” which is herein incorporated by reference in its entirety.

This application may be related to U.S. patent application Ser. No. 13/426,424, filed Mar. 21, 2012, titled “ULTRASOUND NEUROMODULATION TREATMENT OF DEPRESSION AND BIPOLAR DISORDER,” Publication No. US 2012-0283502 A1, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

Described herein are systems and methods for neuromodulation of one or more superficial- or deep-brain targets using more than one means of neuromodulation to up-regulate and/or down-regulate neural activity.

### BACKGROUND

It has been demonstrated that a variety of methods can be employed to neuromodulate superficial or deep brain neural structures. Examples are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, functional stimulation, or drugs. If neural activity is increased or excited, the neural structure is said to be up-regulated; if neural activated is decreased or inhibited, the neural structure is said to be down-regulated. Neural structures are usually assembled in circuits. For example, nuclei and tracts connecting them make up a neural circuit.

Deep Brain Stimulation (DBS) involves implanted electrodes placed within the brain. Typically connecting leads are run down to another part of the body, such as the abdomen where they are connected to the DBS programmer (e.g., Mayberg, H S, Lozano A M, Voon V, McNeely H E, Seminowicz D, Hamani C, Schwalb J M, and S H Kennedy, “Deep brain stimulation for treatment-resistant depression”. Neuron. 45(5):651-60, Mar. 3, 2005).

Transcranial Magnetic Stimulation (TMS) involves electromagnet coils which are powered by brief stimulator pulses (e.g., George M S, Wassermann E M, Williams W, et al., “Changes in mood and hormone levels after rapid-rate transcranial magnetic stimulation of the prefrontal cortex,” J Neuropsychiatry Clin Neuro 1996; 8:172-180; Mishelevich and Schneider, “Trajectory-Based Deep-Brain Stereotactic Transcranial Magnetic Stimulation,” International Application Number PCT/US2007/010262, International Publication Number WO 2007/130308, Nov. 15, 2007).

Ultrasound stimulation is accomplished with focused transducers (e.g., Bystritsky, “Methods for Modifying Electrical Currents in Neuronal Circuits,” U.S. Pat. No. 7,283,861, Oct. 16, 2007).

Radiosurgery involves permanent change to neural structures by applying focused ionizing radiation in such a way that tissue and thus function are modified but without destroying tissue. A quantity of 60 to 80 grey is typically applied at rates on the order of 5 Gy per minute (e.g., Schneider, Adler, Borchers, “Radiosurgical Neuromodulation Devices, Systems, and Methods for Treatment of Behavioral Disorders by External Application of Ionizing Radiation,” U.S. patent application Ser. No. 12/261,347, Publication No.” US2009/0114849, May 7, 2009).

Transcranial Direct Current Stimulation (tDCS) uses electrode pads external to the scalp that depolarize or hyperpolarize neural membranes on the underlying cortex (e.g., Nitsche, M A, and W. Paulus, “Excitability changes induced in the human motor cortex by weak transcranial direct current stimulation,” J. Physiology, 527.3, 633-639, 2000).

Radio-Frequency (RF) stimulation utilizes RF energy as opposed to ultrasound (e.g., Deisseroth & Schneider, “Device and Method for Non-Invasive Neuromodulation,” U.S. patent application Ser. No. 12/263,026, Pub. No.: US2009/0112133. Apr. 30, 2009).

Vagus nerve stimulation involves a programmer in the upper left chest, under the clavicle, with leads wrapped around the vagus nerve with brain stimulation occurring by the vagus connections to brain structures (e.g., George, M., Sackheim, A J, Rush, et al., “Vagus Nerve Stimulation: A New Tool for Brain Research and Therapy,” Biological Psychiatry, 47, 287-295, 2000). Multiple mechanisms have been proposed for the Cyberonics Vagus Nerve Stimulation system for the modulation of mood. These include alteration of norepinephrine release by projections of solitary tract to the locus coeruleus, elevated levels of inhibitory GABA related to vagal stimulation and inhibition of aberrant cortical activity by the reticular activating system (Ghanem T, Early S V, “Vagal nerve stimulator implantation: an otolaryngologist's perspective,” Otolaryngol Head Neck Surg 2006; 135(1):46-51).

Optical stimulation involves methods for stimulating target cells using a photosensitive protein that allows the target cells to be stimulated in response to light (e.g., Zhang, Deisseroth, Mishelevich, and Schneider, “System for Optical Stimulation of Target Cells,” PCT/US2008/050627, International Publication Number WO 2008/089003, Jul. 24, 2008).

Functional stimulation can be accomplished by voluntary movement, induction of sensory input (e.g., pain or pressure) or electrical such as median nerve stimulation (Sailer, Alexandra, G. F. Molnar, D. I. Cunic and Robert Chen, “Effects of peripheral sensory input on cortical inhibition in humans,” Journal of Physiology, 544.2:617-629, 2002).

Drugs can be used for central nervous system effects as well.

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. Down regulation means that the firing rate of the neural target has its firing rate decreased and thus is inhibited and up regulation means that the firing rate of the neural target has its firing rate increased and thus is excited. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2): 179-92. and S. J. Norton, “Can ultrasound be used to stimulate nerve tissue?,” BioMedical Engineering OnLine 2003, 2: 6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42° C. (say in the range of 39 to 42° C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels which resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound mediated opening of calcium channels was also observed by Tyler and colleagues. The above approach is incorporated in a patent application submitted by Tyler (Tyler, William, James P., PCT/US2009/050560, WO 2010/009141, published Jan. 21, 2011).

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap placed over the skull to affect a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med, Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as typically below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

An alternative approach is described by Deisseroth and Schneider (U.S. patent application Ser. No. 12/263,026 published as US 2009/0112133 A1, Apr. 30, 2009) in which modification of neural transmission patterns between neural structures and/or regions is described using sound (including use of a curved transducer and a lens) or RF. The impact of Long-Term Potentiation (LTP) and Long-Term Depression (LTD) for durable effects is emphasized. It is noted that sound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2):275-83 and Clement G T, Hynynen K (2002) A non-invasive method for focusing ultrasound through the human skull. Phys Med Biol 47: 1219-1236.). Ultrasound can be focused to 0.5 to 2 mm as compared to TMS that can be focused to 1 cm at best.

One or a plurality of neural elements can be neuromodulated.

As mentioned, potential application of ultrasonic therapy of deep-brain structures has been covered previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009).

Transcranial Magnetic Stimulation (TMS) has been used for characterization of the motor system. TMS stimulation of the motor cortex is employed to see the motor response in the periphery. The response can be in alternative ways such as Motor Evoked Potentials (MEPs) or measurement of mechanical output. One application is the measurement of conduction time from central to peripheral loci, which can have diagnostic significance. Another is the demonstration of the degree of functional connectivity between the loci. Stimulation more distally such as in the spinal cord nerve roots or the spinal cord itself to measure connectivity from the spinal cord to the periphery. Irrespective of the point of stimulation with the central nervous system, an application is the monitoring of the level of anesthesia present.

While motor-system functions performed using TMS are valuable, they use expensive units, typically costing on the order of $50,000 in 2010 that are large, take a relatively high power, require cooling of the electromagnet stimulation coils, and may be noisy. It would be highly beneficial to be able to perform the same functions using lower-cost stimulation mechanism.

Potential application of ultrasonic therapy of deep-brain structures has been covered previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). It was noted that monophasic ultrasound pulses are more effective than biphasic ones.

Patent applications have been filed addressing neuromodulation of deep-brain targets (Bystritsky, “Methods for modifying electrical currents in neuronal circuits,” U.S. Pat. No. 7,283,861, Oct. 16, 2007 and Deisseroth, K. and M. B. Schneider, “Device and method for non-invasive neuromodulation,” U.S. patent application Ser. No. 12/263,026 published as US 2009/0112133 A1, Apr. 30, 2009).

While the ultrasonic frequencies for neural stimulation are known, it would be preferable to use macro- and micro-pulse shapes optimized for neuromodulation.

Targeting can be done with one or more of known external landmarks, an atlas-based approach (e.g., Tailarach or other atlas used in neurosurgery) or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Cingulate Gyms, are elongated and will be more effectively served with an elongated ultrasound field at the target.

It would be preferable to not only stimulate single or multiple targets synchronously, but to have patterns applied both to a single ultrasound transducer and to the stimulation relationships among multiple such transducers.

As mentioned, it has been demonstrated that focused ultrasound directed at neural structures can stimulate those structures. If neural activity is increased or excited, the neural structure is up regulated; if neural activated is decreased or inhibited, the neural structure is down regulated. Preliminary clinical work by universities (Ben-Gurion University and the University of Rome) using Brainsway Transcranial Magnetic Stimulation (TMS) systems has shown that deep-brain neuromodulation can open up the blood-brain barrier to allow more effective penetration of drugs (e.g., for the treatment of malignant tumors). Ultrasound would be more effective for this purpose because of its higher resolution and thus more specificity. The equipment also costs less and can be portable for use in a variety of settings, including within the home of the patient.

Because of the utility of ultrasound in the neuromodulation of deep-brain structures, application of those techniques to alteration of the permeability of the blood-brain barrier is both logical and desirable even though the target is the blood-brain barrier and not necessarily involving the neuromodulation of the neural target itself.

The power needed for stimulation of the spinal cord is significantly less than needed for deep-brain neuromodulation. Alternative mechanisms for the effects of ultrasound may be discovered as well. In fact, multiple mechanisms may come into play, but, in any case, this would not effect this invention.

Other approaches for delivering focused ultrasound have also been proposed. Bystritsky (U.S. Pat. No. 7,283,861) describes the delivery of focused ultrasound pulses (FUP) produced by multiple ultrasound transducers (said preferably to number in the range of 300 to 1000) arranged in a cap place over the skull to provide a multi-beam output. These transducers are coordinated by a computer and used in conjunction with an imaging system. The user interacts with the computer to direct the FUP to the desired point in the brain, sees where the stimulation actually occurred by viewing the image, and can adjust the position of the FUP accordingly. A position of focus is obtained by adjusting the phases and amplitudes of the ultrasound. The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 500 Hz.) are inhibitory. High frequencies (defined as being in the range of 500 Hz to 5 MHz) are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging

Methods and systems for delivering ultrasound energy to neural targets with mechanical perturbation are described in applicant's earlier patent publications including US2011/0208094; US2011/0190668; and US2011/0270138.

The treatment of neuropathic pain has been demonstrated using electrical spinal cord stimulation (SCS) using electrodes to suppress hyperexcitability of the neurons via alteration of dorsal horn neurochemistry including the release of serotonin, Substance P, and GABA. For treatment of ischemic pain, it has been suggested that the oxygen supply may berestored via sympathetic stimulation and/or vasodilation.

Although it has been demonstrated that focused ultrasound directed at neural structures can stimulate those structures, the prior methods and apparatus have lead to less than ideal results in at least some instances.

If neural activity is increased or excited, the neural structure is up regulated; if neural activated is decreased or inhibited, the neural structure is down regulated. Neural structures are usually assembled in circuits. For example, nuclei and tracts connecting them make up a circuit.

The effect of ultrasound on neural activity appears to be at least two fold. Firstly, increasing temperature will increase neural activity. Secondly, mechanical perturbation appears to be related to the opening of ion channels related to neural activity.

With regards to increasing temperature, an increase up to 42 degrees C. (say in the range of 39 to 42 degrees C.) locally for short time periods will increase neural activity in a way that one can do so repeatedly and be safe. For clinical uses, one needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel.

As mentioned above, with regards to mechanical perturbation, an explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)), in which publication voltage gating of sodium channels in neural membranes was demonstrated. Pulsed ultrasound was found to cause mechanical opening of the sodium channels that resulted in the generation of action potentials. Their stimulation is described as Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm2 upper limit established by the U.S. Food and Drug Administration (FDA) for womb-scanning sonograms; thus such devices should be safe to use on patients. Ultrasound impact to open calcium channels has also been suggested. Tyler incorporated this approach in two patent applications he submitted (Tyler, William, James P., PCT/US2009/050560, WO 2010/009141, “Methods and Devices for Modulating Cellular Activity Using Ultrasound,” published 2011-01-21 and “Devices and Methods for Modulating Brain Activity,” PCT/US2010/055527, WO 2011/057028, published 2011-05-12). Alternative mechanisms for the effects of ultrasound may be discovered as well. In fact, multiple mechanisms may come into play.

Approaches to date of delivering focused ultrasound vary, and the clinical results and predictability can be less than ideal in at least some instances. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output. The position of focus may be obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits.

Deisseroth and Schneider (U.S. patent application Ser. No. 12/263,026 published as US 2009/0112133 A1, Apr. 30, 2009) describe an alternative approach in which modifications of neural transmission patterns between neural structures and/or regions are described using ultrasound (including use of a curved transducer and a lens) or RF. The impact of Long-Term Potentiation (LTP) and Long-Term Depression (LTD) for durable effects is emphasized. It is noted that ultrasound produces stimulation by both thermal and mechanical impacts.

Many patients suffer from diseases and conditions that may be less than ideally treated. For example, patient conditions having similar symptoms can make it difficult to determine the underlying cause of the patient's symptoms. Also, at least some therapies may provide less than ideal results in at least some instances, and it would be helpful to use presently available therapies more effectively.

Because of the utility of ultrasound in the neuromodulation of neurological structures such as deep-brain structures, it would be both beneficial and desirable to provide improved diagnosis of patient conditions and improved treatment planning. Further, because of the utility of ultrasound in the neuromodulation of deep-brain structures and the need for flexibility in delivery of the energy in different circumstances considering the given condition for which the neuromodulation is being applied and the specific patient, it is both logical and desirable to apply the neuromodulation in sessions.

### SUMMARY OF THE DISCLOSURE

In general, described herein are systems, devices and methods, including software, hardware, firmware, and the like, for neuromodulation. This disclosure is broken up into twelve parts or sections, summarized below, which may be understood individually, and also in context with one or more other parts. Thus, although this disclosure is divided into different parts or sections illustrating a variety of different devices, systems and methods, any of the information contained in one or more of the other sections may be applied to any of the other sections, individually or collectively. Alternatively, each section may be considered independent of the other sections.

For example, described herein are systems and methods for Ultrasound Neuromodulation including one or more ultrasound sources for neuromodulation of target deep brain regions to up-regulate or down-regulate neural activity.

Also described herein are systems and methods for control of Ultrasonic Stimulation including one or a plurality ultrasound sources for neuromodulation of target deep brain regions to up-regulate or down-regulated neural activity.

Also described herein are systems and methods for Ultrasound Stimulation including one or a plurality of ultrasound sources for stimulation of target deep brain regions to up-regulate or down-regulated neural activity.

Also described herein are systems and methods for treatment planning for ultrasound neuromodulation and other treatment modalities for up-regulation or down-regulation of neural activity.

Also described herein are systems and methods for Ultrasound Neuromodulation of the occipital nerve and related neural structures.

Also described herein are systems and methods for ultrasound neuromodulation of the brain and other neural structures.

Also described herein are systems and methods for Ultrasound Neuromodulation including one or a plurality of ultrasound sources for stimulation of target deep brain regions to up-regulate or down-regulate neural activity.

Also described herein are systems and methods for Ultrasound Stimulation including one or a plurality of ultrasound sources for stimulation of target deep brain regions to up-regulate or down-regulate neural activity.

Also described herein are systems and methods for using ultrasound-neuromodulation techniques for the treatment of medical conditions.

Also described herein are methods and systems for neuromodulation and more particularly to methods and systems for neuromodulation of a patient's spinal cord for treatment of pain and other conditions.

Also describe herein are systems and methods for neuromodulation and more particularly to systems and methods for diagnosis and treatment with ultrasound.

### Summary of Part I: Multi-Modality Neuromodulation of Brain Targets

In some variations, is the purpose of this invention to provide methods and systems for non-invasive deep brain or superficial stimulation using multiple modalities simultaneously or on an interleaved basis. This approach is particularly of benefit because impacting multiple points in a neural circuit to produce Long-Term Potentiation (LTP) or Long-Term Depression (LTD). Multiple modalities considered are deep-brain stimulators (DBS) with implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation (VNS), functional stimulation, and drugs. Note that VNS is representative of other implanted modalities where nerves located outside the cranium are stimulated and these other implanted modalities are covered by this invention. An example is stimulation of the sphenopalatine ganglion to abort a migraine headache.

For example, described herein are methods of modulating deep-brain targets using multiple therapeutic modalities, the method comprising: applying a plurality of therapeutic modalities to a deep-brain target, applying power to each of the on-line therapeutic modalities via a control circuit thereby neuromodulating the activity of the deep brain target regions, and working in coordination with the off-line therapeutic modalities.

The therapeutic modalities are selected from the group may consist of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

In some variations, a therapy is selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, functional stimulation, and drugs is combined one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation other-implant stimulation, functional stimulation, drugs.

The disorder may be treated by neuromodulation, the method comprising modulating the activity of one target brain region or simultaneously modulating the activity of two or more target brain regions, wherein the target brain regions are selected from the group consisting of NeoCortex, any of the subregions of the Pre-Frontal Cortex, Orbito-Frontal Cortex (OFC), Cingulate Genu, subregions of the Cingulate Gyrus, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

In some variations, the disorder treated is selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, epilepsy.

In some variations, the multi-modality therapy is applied for the purpose selected from the group consisting of cognitive enhancement, hedonic stimulation, enhancement of neural plasticity, improvement in wakefulness, brain mapping, diagnostic applications, and other research functions.

In some variations, the one or a plurality of targets are hit by a plurality of therapeutic modalities.

In some variations, a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring.

In some variations, the output is on-line, real time where neuromodulation parameters are changed immediately under direct control of the Treatment Planning and Control System.

In some variations, the on-line, real-time neuromodulators are selected from the group consisting of ultrasound transducers, TMS stimulators.

In some variations, the output is on-line prescriptive where neuromodulation parameters are directly set in programmers and the effect is both reversible and seen immediately.

In some variations, the on-line, prescriptive neuromodulators are selected from the group consisting of on-line, real-time programmable DBS programmers, Vagus Nerve Stimulation programmers, neuromodulators with similar characteristics to DBS programmers, Vagus Nerve Stimulation programmers, other-implant programmers.

In some variations, the output is off-line prescriptive adjustable where instructions are generated for users to adjust programmers and the effect is reversible but the effect is seen at a later time after the programmers have been so adjusted.

In some variations, the off-line, prescriptive adjustable neuromodulators are selected from the group consisting of off-line prescriptive adjustable DBS programmers, Vagus Nerve Stimulation programmers, other-implant programmers, neuromodulators with similar characteristics to DBS programmers, Vagus Nerve Stimulation programmers other-implant programmers.

In some variations, the output is off-line prescriptive permanent where neuromodulation parameters are instructions are generated for users to adjust parameters and the effect is not reversible and the effect is seen at a later time after the change has been made.

In some variations, the off-line, prescriptive permanent neuromodulators are selected from the group consisting of radiosurgery, neuromodulators with characteristics similar to radiosurgery.

In some variations, the treatment planning and control system varies, as applicable, a plurality of elements selected from the group consisting of direction of energy emission, intensity, pulse-train duration, session durations, numbers of sessions, frequency, phase, firing patterns, number of sessions, relationship to other controlled modalities.

In some variations, real-time modalities are applied simultaneously.

In some variations, real-time modalities are applied sequentially.

In some variations, multiple indications are treated simultaneously or sequentially.

In some variations, the multiple conditions have one or more common targets.

In some variations, the multiple conditions have no common targets.

Also described herein are methods of modulating deep-brain targets using multiple therapeutic modalities for the treatment of pain, the method comprising: applying down-regulation via ultrasound to the Dorsal Anterior Cingulate Gyrus, applying down-regulation via ultrasound to the Cingulate Genu, applying down-regulation via Transcranial Magnetic Stimulation to the Insula, applying down-regulation via ultrasound to the Caudate Nucleus, and applying down-regulation via Deep Brain Stimulation of the Thalamus.

In some variations, a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

In some variations, alternative targets in an applicable neural circuit are substituted.

Also described herein are methods of modulating deep-brain targets using multiple therapeutic modalities for the treatment of depression, the method comprising: applying down-regulation via ultrasound to the Orbito-Frontal Cortex, applying up-regulation via ultrasound to the Dorsal Anterior Cingulate Gyrus, applying down-regulation via ultrasound to the Subgenu Cingulate, applying down-regulation via ultrasound to the Cingulate Genu, applying up-regulation via Transcranial Magnetic Stimulation to the right Insula, applying down-regulation via Transcranial Magnetic Stimulation to the left Insula, applying up-regulation via Deep Brain Stimulation to the Nucleus Accumbens, applying up-regulation via ultrasound to the Caudate Nucleus, applying down-regulation via radiosurgery of the Amygdala, and applying down-regulation via Deep Brain Stimulation of the Thalamus.

In some variations, a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

In some variations, alternative targets in an applicable neural circuit are substituted.

Also described herein are methods of modulating deep-brain targets using multiple therapeutic modalities for the treatment of addiction, the method comprising: applying down-regulation via ultrasound to the Orbito-Frontal Cortex, applying up-regulation via ultrasound to the Dorsal Anterior Cingulate Gyrus, applying down-regulation via Transcranial Magnetic Stimulation to the Insula, applying down-regulation via radiosurgery of the Nucleus Accumbens, and applying down-regulation via Deep Brain Stimulation of the Globus Pallidus.

In some variations, a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

In some variations, alternative targets in an applicable neural circuit are substituted.

Also described herein are methods of modulating deep-brain targets using multiple therapeutic modalities for the treatment of obesity, the method comprising: applying down-regulation via Transcranial Magnetic Stimulation of the Orbito-Frontal Gyrus, applying down-regulation via ultrasound to the Hypothalamus, applying down-regulation via Transcranial Magnetic Stimulation to the Insula, and applying down-regulation via radiosurgery of the Lateral Hypothalamus.

In some variations, a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

In some variations, alternative targets in an applicable neural circuit are substituted.

Also described herein are methods of modulating deep-brain targets using multiple therapeutic modalities for the treatment of epilepsy, the method comprising: applying down-regulation via Transcranial Magnetic Stimulation of the Temporal Lobe, applying down-regulation via radiosurgery of the Amygdala, applying down-regulation via ultrasound to the Hippocampus, applying up-regulation via Vagus Nerve Stimulation of the Thalamus, and applying down-regulation via Deep Brain Stimulation of the Cerebellum.

In some variations, a therapy selected from the group consisting of implanted deep-brain stimulation (DBS) using implanted electrodes, Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, and drugs is replaced by one or more therapies selected from the group consisting of are implanted deep-brain stimulators (DBS), Transcranial Magnetic Stimulation (TMS), transcranial Direct Current Stimulation (tDCS), implanted optical stimulation, focused ultrasound, radiosurgery, Radio-Frequency (RF) stimulation, vagus nerve stimulation, other-implant stimulation, functional stimulation, drugs.

In some variations, alternative targets in an applicable neural circuit are substituted.

Thu, disclosed are methods and systems and methods for deep or superficial deep-brain stimulation using multiple therapeutic modalities. These impact multiple points in a neural circuit or one or multiple points in multiple neural circuits to produce Long-Term Potentiation (LTP) or Long-Term Depression (LTD) to treat indications such as neurologic and psychiatric conditions. Modality examples are implanted deep-brain stimulators (DBS), Transcranial Magnetic Stimulation (TMS), transcranial Direct Current Stimulation (tDCS), implanted optical stimulation, focused ultrasound, RF stimulation, vagus nerve stimulation, other-implant stimulation, functional stimulation, and drugs. Some targets may be up-regulated and others down-regulated. Coordinated control is provided, as applicable, for control of the direction of the energy emission, intensity, session duration, frequency, pulse-train duration, phase, and numbers of sessions, if and as applicable, for neurormodulation of neural targets. Use of ancillary monitoring or imaging to provide feedback may be applied.

### Summary of Part II: Neuromodulation of Deep-Brain Targets Using Focused Ultrasound

It is the purpose of this invention to provide methods and systems for non-invasive deep brain or superficial neuromodulation using ultrasound impacting one or multiple points in a neural circuit to produce acute effects on Long-Term Potentiation (LTP) or Long-Term Depression (LTD). Sonic transducers are positioned by spinning them around the head on a track with under control of direction of the energy emission, control of intensity for up-regulation or down-regulation, and control of frequency and phase for focusing on neural targets. The transducer may also rotate while it is moving around the track to enhance ultrasound targeting and delivery. Alternatively the ultrasound transducers may be fixed to the track. Use of ancillary monitoring or imaging to provide feedback is optional. In embodiments were concurrent imaging is to be done, the device of the invention is to be constructed of non-ferrous material. The apparatus can also be optionally covered by a shell.

As mentioned, targeting can be done with one or more of known external landmarks, an atlas-based approach (e.g., Tailarach or other atlas used in neurosurgery) or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Cingulate Gyrus, are elongated and will be more effectively served with an elongated ultrasound field at the target.

For example, described herein are methods of neuromodulating one or a plurality of deep-brain targets using ultrasound stimulation, the method comprising: aiming one or a plurality of ultrasound transducers at one or a plurality of deep-brain targets, applying power to each of the ultrasound transducers via a control circuit thereby neuromodulating the activity of the deep brain target region, moving one or a plurality of transducers around a track surrounding the mammal's head.

In some variations, the method further comprises identifying a deep-brain target.

In some variations, the method further comprises where neuromodulation of a plurality of targets is selected from the group consisting of up-regulating all neuronal targets, down-regulating all neuronal targets, up-regulating one or a plurality of neuronal targets and down-regulating the other targets.

In some variations, the step of aiming comprising orienting the ultrasound transducer and focusing the ultrasound so that it hits the target.

In some variations, the acoustic ultrasound frequency is in the range of 0.3 MHz to 0.8 MHz.

In some variations, the power applied is selected from group consisting of less than 180 mW/cm.sup.2 and greater than 180 mW/cm.sup.2 but less than that causing tissue damage.

In some variations, a stimulation frequency of 300 Hz or lower is applied for inhibition of neural activity.

In some variations, the stimulation frequency is in the range of 500 Hz to 5 MHz for excitation.

In some variations, the focus area of the pulsed ultrasound is selected from the group consisting of 0.5 to 500 mm in diameter and 500 to 1500 mm in diameter.

In some variations, the number of ultrasound transducers is between 1 and 25.

In some variations, the disorder is treated by neuromodulation, wherein the target brain regions are selected from the group consisting of NeoCortex, any of the subregions of the Pre-Frontal Cortex, Orbito-Frontal Cortex (OFC), Cingulate Genu, subregions of the Cingulate Gyrus, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

In some variations, the disorder treated is selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

In some variations, the ultrasound is applied for the purpose selected from the group consisting of cognitive enhancement, hedonic stimulation, enhancement of neural plasticity, improvement in wakefulness, brain mapping, diagnostic applications, and other research functions.

In some variations, mechanical perturbations are applied radially or axially to move the ultrasound transducers.

In some variations, a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

In some variations, ultrasound therapy is combined with one or more therapies selected from the group consisting of Radio-Frequency (RF) therapy, Transcranial Magnetic Stimulation (TMS), transcranial Direct Current Stimulation (tDCS), Deep Brain Stimulation (DBS) using implanted electrodes.

In some variations, one or a plurality of ultrasound transducers moving around a track surrounding the mammal's had are rotated as they go around the track to maintain focus for a longer period of time.

In some variations, the position of one or a plurality of ultrasound transducers are mounted on the track surrounding the mammal's head in a fixed position.

In some variations, there are contradictory effects relative to clinical indications, the method comprising: a. identifying other targets in the neural circuits that impact those clinical indications that are not in common, and b. up-regulating or down-regulating one or a plurality of those targets, whereby the contradictory effects are minimized.

In some variations, ultrasound therapy is replaced with one or more therapies selected from the group consisting of Radio-Frequency (RF) therapy, Transcranial Magnetic Stimulation (TMS), transcranial Direct Current Stimulation (tDCS), Deep Brain Stimulation (DBS) using implanted electrodes.

Thus, disclosed are methods and systems for non-invasive deep brain or superficial neuromodulation for up-regulation or down-regulation using ultrasound impacting one or multiple points in a neural circuit to produce Long-Term Potentiation (LTP) or Long-Term Depression (LTD) to treat indications such as neurologic and psychiatric conditions. Ultrasound transducers are positioned by spinning them around the head on a track, as well as individually rotated or not, with control of direction of the energy emission, intensity, frequency, and phase/intensity relationships to targeting and accomplishing up-regulation and/or down-regulation. Alternatively the ultrasound transducers may be at fixed locations on the track. Use of ancillary monitoring or imaging to provide is optional.

### Summary of Part III: Patient Feedback for Control of Ultrasound Deep-Brain Neuromodulation

It is the purpose of this invention to provide methods and systems and methods for patient feedback control of non-invasive deep brain or superficial neuromodulation using ultrasound impacting one or multiple points in a neural circuit to produce acute effects and, with application in multiple sessions, Long-Term Potentiation (LTP) or Long-Term Depression (LTD). One or more of ultrasound transducer positioning, frequency, intensity, and phase/intensity relationships are changed through feedback from the patient to optimize the patient experience through up-regulation or down regulation. Examples are decreases in acute pain or tremor due to more effective impact on the neural targets.

For example, described herein are methods of modulating a deep-brain targets using ultrasound neuromodulation, the method comprising: a mechanism for aiming one or a plurality of ultrasound transducers at one or more a deep-brain targets; applying power to each of the ultrasound transducers via a control circuit thereby modulating the activity of the deep brain target region; providing a mechanism for feedback from the patient based on the acute sensory or motor conditions of the patient; and using that feedback to control one or more parameters to maximize the desired effect.

In some variations, the method further comprises neuromodulation in a manner selected from the group of up-regulation, down-regulation.

In some variations, the means of control is orienting one or a plurality of ultrasound transducers.

In some variations, the means of control is adjusting the pulse frequency of one or a plurality of ultrasound transducers.

In some variations, the means of control is adjusting the phase/intensity relationships within and among the plurality of ultrasound transducers.

In some variations, the means of control is adjusting the intensity relationships within an ultrasound transducer or among a plurality of ultrasound transducers.

In some variations, the means of control is adjusting the fire patterns within an ultrasound transducer or among a plurality of ultrasound transducers.

In some variations, the means of control is adjusting the dynamic sweeps of a dynamic ultrasound transducer or a plurality of dynamic ultrasound transducers.

In some variations, the acoustic ultrasound frequency is in the range of 0.3 MHz to 0.8 MHz.

In some variations, the power applied is less than 180 mW/cm².

In some variations, the power applied is greater than 180 mW/cm² but less than that causing tissue damage.

In some variations, a stimulation frequency for of 300 Hz or lower is applied for inhibition of neural activity.

In some variations, the stimulation frequency for excitation is in the range of 500 Hz to 5 MHz.

In some variations, the focus area of the pulsed ultrasound is 0.5 to 1500 mm in diameter.

In some variations, one effect is used as a surrogate for another effect.

In some variations, the first effect is acute pain and the second effect is chronic pain.

In some variations, a disorder is treated by neural neuromodulation, the method comprising modulating the activity of one target brain region or simultaneously modulating the activity of a plurality target brain regions, wherein the target brain regions are selected from the group consisting of NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

In some variations, the disorder treated is selected from the group consisting of pain, Parkinson's Disease, depression, bipolar disorder, tinnitus, addiction, OCD, Tourette's Syndrome, ticks, cognitive enhancement, hedonic stimulation, diagnostic applications, and research functions.

In some variations, Transcranial Magnetic Stimulation coils are used in place or ultrasound transducers.

In some variations, the feedback control of ultrasound transducers is combined with the application, with or without feedback control, of one or more other modalities selected from the group of deep-brain stimulators (DBS) using implanted electrodes, Transcranial Magnetic Stimulation (TMS), transcranial Direct Current Stimulation (tDCS), implanted optical stimulation, stereotactic radiosurgery, Radio-Frequency (RF) stimulation, vagus nerve stimulation, or functional stimulation.

Thus, disclosed are methods and systems and methods for patient-feedback control of non-invasive deep brain or superficial neuromodulation using sound impacting one or multiple points in a neural circuit to produce acute effects and, with application in multiple sessions, Long-Term Potentiation (LTP) or Long-Term Depression (LTD) to treat indications such as neurologic and psychiatric conditions. One or more of sonic transducer positioning, intensity, frequency, dynamic sweeps, phase/intensity relationships, and firing patterns are changed through feedback from the patient to optimize patient experience through up-regulation or down regulation. Examples are decreases in acute pain or tremor due to more effective impact on the neural targets.

### Summary of Part IV: Shaped and Steered Ultrasound for Deep-Brain Neuromodulation

It is the purpose of this invention to provide a device for producing shaped or steered ultrasound for non-invasive deep brain or superficial stimulation impacting one or a plurality of points in a neural circuit to produce acute effects or Long-Term Potentiation (LTP) or Long-Term Depression (LTD) using up-regulation or down-regulation.

For example, described herein are ultrasound transducers for neuromodulation of a deep-brain target comprising: a. an ultrasound-generation array with a curvature matched to the depth of the target, and b. a shape matched to the shape of the target, whereby said ultrasound transducer neuromodulates the targeted neural structures producing regulation selected from the group consisting of up-regulation and down-regulation.

In some variations, the ultrasound transducer is elongated to match an elongated target.

In some variations, the ultrasound transducer is a hemispheric cup shaped to match a point target.

In some variations, a plurality of ultrasound transducers are employed to neuromodulate targets selected from the group consisting of multiple targets in a single neural circuit and multiple targets in multiple neural circuits.

In some variations, one or plurality of ultrasound transducers are used with one or a plurality of controlled elements selected from the group consisting of direction of the energy emission, intensity, frequency, firing patterns, and phase/intensity relationships for beam steering and focusing on neural targets.

Also described herein are ultrasound transducers for neuromodulation of a deep-brain target comprising: a. an ultrasound-generation array, and b. a separate lens shape matched to the depth and shape of the target, whereby said ultrasound transducer neuromodulates the targeted neural structures producing regulation selected from the group consisting of up-regulation and down-regulation.

In some variations, the separate lens used in conjunction with an ultrasound-generating transducer array used in conjunction with the Transcranial Magnetic Stimulation electromagnet has an attachment selected from the group consisting of the bonded to the ultrasound-generating transducer array and not bonded to the ultrasound-generating transducer array.

In some variations, the separate lens used in conjunction with the ultrasound generator is interchangeable.

In some variations, the separate lens is elongated to match an elongated target.

In some variations, the separate ultrasound lens is a hemispheric cup shaped to match a point target.

Also described herein are ultrasound transducers for neuromodulation of a deep-brain target comprising: a. a flat ultrasound-generation array, b. an ultrasound controller generating varying the phase/intensity relationships to steer and shape the ultrasound beam, whereby said ultrasound transducer neuromodulates the targeted neural structures producing regulation selected from the group consisting of up-regulation and down-regulation.

In some variations, the ultrasound transducer has a curved ultrasound-generation array instead of a flat ultrasound-generation array.

In some variations, one or plurality of ultrasound transducers are used with one or a plurality of controlled elements selected from the group consisting of direction of the energy emission, intensity, frequency, firing patterns, and phase/intensity relationships for beam steering and focusing on neural targets.

Also described herein are systems for neuromodulation of a deep-brain target comprising: a. an ultrasound-generation array with a curvature and shaped matched to the depth and shape of the target, and b. a Transcranial Magnetic Stimulation electromagnet, whereby said combination ultrasound transducer and Transcranial Magnetic Stimulation electromagnet neuromodulates the targeted neural structures producing regulation selected from the group consisting of up-regulation and down-regulation.

In some variations, the separate lens used in conjunction with an ultrasound-generating transducer array used in conjunction with the Transcranial Magnetic Stimulation electromagnet has an attachment selected from the group consisting of the bonded to the ultrasound-generating transducer array and not bonded to the ultrasound-generating transducer array.

In some variations, the separate lens used in conjunction with the ultrasound-generating array that is used in conjunction with the Transcranial Magnetic Stimulation electromagnet is interchangeable.

In some variations, a plurality of combination ultrasound-generating transducer arrays and Transcranial Magnetic Stimulation electromagnets are employed to neuromodulate targets selected from the group consisting of multiple targets in a neural circuit and multiple targets in multiple neural circuits.

In some variations, the combination ultrasound-generating transducer arrays and Transcranial Magnetic Stimulation electromagnets are used with control for the ultrasound-generating transducer arrays of one or a plurality of control elements selected from the group consisting of direction of the energy emission, control of intensity, control of frequency for regulation selected from the group consisting of up-regulation and down-regulation, and control of phase/intensity relationships for beam steering and focusing on neural targets

In some variations, the control for the Transcranial Magnetic Stimulation are one or a plurality of control elements selected from the group consisting of intensity, frequency, pulse shape, and timing patterns of the stimulation of the Transcranial Magnetic Stimulation electromagnets.

In some variations, the combination of a Transcranial Magnetic Stimulation stimulation means and a coaxial ultrasound transducer array aimed at a neural target increases the neuromodulation of the target to a greater degree than obtainable by either means used alone.

Thus, disclosed are devices for producing shaped or steered ultrasound for non-invasive deep brain or superficial neuromodulation impacting one or a plurality of points in a neural circuit. Depending on the application this can produce short-term effects (as in the treatment of post-surgical pain) or long-term effects in terms of Long-Term Potentiation (LTP) or Long-Term Depression (LTD) to treat indications such as neurologic and psychiatric conditions. The ultrasound transducers are used with control of direction of the energy emission, control of intensity, control of frequency for up-regulation or down-regulation, and control of phase/intensity relationships for focusing on neural targets.

### Summary of Part V: Treatment Planning for Deep-Brain Neuromodulation

The invention provides methods and systems for treatment planning for non-invasive deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Effectiveness of the application of ultrasound and other non-invasive, non-reversible modalities producing deep-brain neuromodulation such as Transcranial Magnetic Stimulation (TMS), transcranial Direct Current Stimulation (tDCS), Radio-Frequency (RF), or functional stimulation can be improved with treatment planning Treatment-plan recommendations for the application of non-reversible and/or invasive modalities such as Deep Brain Stimulation (DBS), stereotactic radiosurgery, optical stimulation, Sphenopalatine Ganglion or other localized stimulation, vagus nerve Stimulation (VNS), or future means of neuromodulation can be included.

Ultrasound transducers or other energy sources are positioned and the anticipated effects on up-regulation and/or down-regulation of their direction of energy emission, intensity, frequency, and phase/intensity relationships, dynamic-sweep configuration, and timing patterns mapped onto treatment-planning targets. The maps of treatment-planning targets onto which the mapping occurs can be atlas (e.g., Tailarach Atlas) based or image (e.g., fMRI or PET) based. Maps may be representative and applied directly or scaled for the patient or may be specific to the patient.

While rough targeting can be done with one or more of known external landmarks, or the landmarks combined with an atlas-based approach (e.g., Tailarach or other atlas used in neurosurgery) or imaging (e.g., fMRI or Positron Emission Tomography), explicit treatment planning adds benefit.

For example, described herein are methods for treatment planning for neuromodulation of deep-brain targets using ultrasound neuromodulation, the method comprising: setting up sets of applications and supported transducer configurations with associated capabilities, executing treatment-planning sessions including setting parameters for the session, system recommendations and user acceptance of changes to applications, targets, up- or down-regulation, stimulation frequencies, iterating through set of applications; iterating through set of targets; iterating through and applying in designated order one or more variables selected from the group consisting of position, intensity, firing-timing pattern, phase/intensity relationships, dynamic sweeps; presenting treatment plan to user who accepts or changes; whereby the treatment to be delivered is tailored to the patient.

In some variations, the one or plurality of treatment modalities are selected from the group consisting of ultrasound, Deep Brain Stimulation, stereotactic radiosurgery, optical stimulation, Sphenopalatine Ganglion stimulation, other localized stimulation, vagus nerve stimulation, and future means of neuromodulation.

In some variations, the maps of treatment-planning targets onto which the mapping are selected from the group consisting of atlas based or image based.

In some variations, the maps are selected from the group consisting of specific to the patient, representative and applied directly, and representative where scaled for the patient.

In some variations, the one or a plurality of target brain regions involved in the treatment plan are selected from the group consisting of NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, and any of the tracts between the brain targets.

In some variations, the one or plurality of disorders for which treatment is planned are selected from the group consisting of: addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

In some variations, the one or a plurality of application for which treatment is planned are selected from the group consisting of cognitive enhancement, hedonic stimulation, enhancement of neural plasticity, improvement in wakefulness, brain mapping, diagnostic applications, and research functions.

Also described herein are systems for treatment planning for neuromodulation of deep-brain targets using ultrasound neuromodulation, the method comprising: setting up sets of applications and supported transducer configurations with associated capabilities, executing treatment-planning sessions including setting parameters for the session, system recommendations and user acceptance of changes to applications, targets, up- or down-regulation, stimulation frequencies, iterating through set of applications; iterating through set of targets; iterating through and applying in designated order one or more variables selected from the group consisting of position, intensity, firing-timing pattern, phase/intensity relationships, dynamic sweeps; presenting treatment plan to user who accepts or changes; whereby the treatment to be delivered is tailored to the patient.

In some variations, the one or plurality of treatment modalities are selected from the group consisting of ultrasound, Deep Brain Stimulation, stereotactic radiosurgery, optical stimulation, Sphenopalatine Ganglion stimulation, other localized stimulation, vagus nerve stimulation, and future means of neuromodulation.

In some variations, the maps of treatment-planning targets onto which the mapping are selected from the group consisting of atlas based or image based.

In some variations, the maps are selected from the group consisting of specific to the patient, representative and applied directly, and representative where scaled for the patient.

In some variations, the one or a plurality of target brain regions involved in the treatment plan are selected from the group consisting of NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, and any of the tracts between the brain targets.

In some variations, the one or plurality of disorders for which treatment is planned are selected from the group consisting of addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

In some variations, the one or a plurality of application for which treatment is planned are selected from the group consisting of: cognitive enhancement, hedonic stimulation, enhancement of neural plasticity, improvement in wakefulness, brain mapping, diagnostic applications, and research functions.

Thus, disclosed are methods and systems for treatment planning for deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Ultrasound transducers or other energy sources are positioned and the anticipated effects on up-regulation and/or down-regulation of their direction of energy emission, intensity, frequency, firing/timing pattern, and phase/intensity relationships mapped onto the recommended treatment-planning targets. The maps of treatment-planning targets onto which the mapping occurs can be atlas (e.g., Tailarach Atlas) based or image (e.g., fMRI or PET) based. Atlas and imaged-based maps may be representative and applied directly or scaled for the patient or may be specific to the patient.

### Summary of Part VI: Ultrasound Neuromodulation of the Brain, Nerve Roots, and Peripheral Nerves

It is the purpose of this invention to provide methods and systems and methods for ultrasound stimulation of the cortex, nerve roots, and peripheral nerves, and noting or recording muscle responses to clinically assess motor function. In addition, just like Transcranial Magnetic Stimulation, ultrasound neuromodulation can be used to treat depression by stimulating cortex and indirectly impacting deeper centers such as the cingulate gyms through the connections from the superficial cortex to the appropriate deeper centers. Ultrasound can also be used to hit those deeper targets directly. Positron Emission Tomography (PET) or fMRI imaging can be used to detect which areas of the brain are impacted. Compared to Transcranial Magnetic Stimulation, Ultrasound Stimulation systems cost significantly less and do not require significant cooling.

For example, described herein are systems of non-invasively neuromodulating the brain using ultrasound stimulation, the system comprising: aiming an ultrasound transducer at superficial cortex, applying pulsed power to said ultrasound transducer via a control circuit thereby neuromodulating the target, whereby results are selected from the group consisting of functional and diagnostic.

In some variations, the plurality of control elements is selected from the group consisting of intensity, frequency, pulse duration, and firing pattern.

In some variations, the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

In some variations, the level ultrasound stimulation is used to assess the excitability of the cortex.

Also described herein are system for non-invasively neuromodulating the brain using ultrasound stimulation, the system comprising: aiming an ultrasound transducer at a neural target, applying pulsed power to said ultrasound transducer via a control circuit thereby stimulating the target, placement of one or a plurality of sensors at a distance from the target, whereby results are selected from the group consisting of diagnostic and monitoring.

In some variations, the plurality of control elements is selected from the group consisting of intensity, frequency, pulse duration, and firing pattern.

In some variations, the time from stimulation to the time of detection is measured at a sensor where the sensor is placed a location selected from the group consisting of spinal-cord nerve root, peripheral nerve and muscle.

In some variations, the system is used for determination of conduction velocity.

In some variations, the system is used for monitoring of the level of anesthesia.

In some variations, the system is used for monitoring of neural function related to spinal cord surgery.

Also described herein are methods of non-invasively neuromodulating the brain using ultrasound stimulation, the method comprising: aiming an ultrasound transducer at superficial cortex, applying pulsed power to said ultrasound transducer via a control circuit thereby neuromodulating the target, whereby results are selected from the group consisting of functional and diagnostic.

In some variations, the plurality of control elements is selected from the group consisting of intensity, frequency, pulse duration, and firing pattern.

In some variations, the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

In some variations, the level ultrasound stimulation is used to assess the excitability of the cortex.

Also described herein are methods of non-invasively neuromodulating the brain using ultrasound stimulation, the system comprising: aiming an ultrasound transducer at a neural target, applying pulsed power to said ultrasound transducer via a control circuit thereby stimulating the target, placement of one or a plurality of sensors at a distance from the target, whereby results are selected from the group consisting of diagnostic and monitoring.

In some variations, the plurality of control elements is selected from the group consisting of intensity, frequency, pulse duration, and firing pattern.

In some variations, the time from stimulation to the time of detection is measured at a sensor where the sensor is placed a location selected from the group consisting of spinal-cord nerve root, peripheral nerve and muscle.

In some variations, the system is used for determination of conduction velocity.

In some variations, the system is used for monitoring of the level of anesthesia.

In some variations, the system is used for monitoring of neural function related to spinal cord surgery.

Thus, disclosed are methods and systems for non-invasive ultrasound neuromodulation of superficial cortex of the brain or stimulation of nerve roots or peripheral nerves. Such stimulation is used for such purposes as determination of motor threshold, demonstrating whether connectivity to peripheral nerves or motor neurons exists and performing nerve conduction-speed studies. Neuromodulation of the brain allows treatment of conditions such as depression via stimulating superficial neural structures that have connections to deeper involved centers. Imaging is optional.

### Summary of Part VII: Ultrasound Macro-Pulse and Micro-Pulse Shapes for Neuromodulation

It is one purpose of this invention to provide methods and systems and methods for optimizing the macro- and micro-pulse shapes used for ultrasound neuromodulation of the brain and other neural structures. Ultrasound neuromodulation is accomplished superimposing pulse trains on the base ultrasound carrier. For example, pulses spaced at 1 Hz of 250 μsec in duration may be superimposed on an ultrasound carrier of 500 kHz. Macro-pulse shaping refers to the overall shaping of the individual pulses delivered at so many Hz (e.g., the pulses appearing at 1 Hz). Micro-pulse shaping refers to the shaping of the individual constituent waveforms in the carrier (e.g., 500 kHz). Either the macro-pulse shapes or the micro-pulse shapes can be sine waves, square waves, triangular waves, or arbitrarily shaped waves. Neither needs to consistent, that is all being the same shape (e.g., all sine waves); heterogeneous mixtures are permitted (e.g., sine waves mixed with square waves) either within the macro or micro or between the macro and micro. Functional output and/or Positron Emission Tomography (PET) or fMRI imaging can judge the results. In addition, the effect on a readily observable function such as stimulation of the palm and assessing the impact on finger movements can be done and the effect of changing of the macro-pulse and/or micro-pulse characteristics observed.

For example, described herein are systems of non-invasively stimulating neural structures such as the brain using ultrasound stimulation, the system comprising: aiming an ultrasound transducer at the selected neural target, macro-shaping the pulse outline of the tone burst, applying pulsed power to said ultrasound transducer via a control circuit thereby whereby the neural structure is neuromodulated.

In some variations, the macro-pulse shape is selected from the group consisting of sine wave, square wave, triangular wave, and arbitrary wave.

In some variations, the macro pulses are selected from the group consisting of homogeneous and heterogeneous.

In some variations, the macro-pulse shape is made up of micro-pulse shapes selected from the group consisting of sine wave, square wave, triangular wave, and arbitrary wave.

In some variations, the micro pulses are selected from the group consisting of homogeneous and heterogeneous.

In some variations, the plurality of control elements is selected from the group consisting of intensity, frequency, pulse duration, and firing pattern.

In some variations, system further comprises focusing the sound field of an ultrasound transducer at the target nerves neuromodulating the activity of the target in a manner selected from the group of up-regulation and down-regulation.

In some variations, the configuration of ultrasound power is selected from the group consisting of monophasic and biphasic.

In some variations, the mechanism for focus of the ultrasound is selected from the group of fixed ultrasound array, flat ultrasound array with lens, non-flat ultrasound array with lens, flat ultrasound array with controlled phase and intensity relationships, and ultrasound non-flat array with controlled phase and intensity relationships.

In some variations, the neuromodulation results in a durable effect selected from the group consisting of Long-Term Potentiation and Long-Term Depression.

In some variations, the disorder treated is selected from the group consisting of addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy.

In some variations, the disorder treated is applied to the group consisting of cognitive enhancement, hedonic stimulation, enhancement of neural plasticity, improvement in wakefulness, brain mapping, diagnostic applications, and research functions.

In some variations, the invention is applied to globally depress neural activity as in the early treatment of head trauma or other insults to the brain.

In some variations, the efficacy of the macro-pulse neuromodulation is judged via an imaging mechanism selected from the group consisting of fMRI, Positron Emission Tomography, and other.

In some variations, the efficacy of the micro-pulse neuromodulation is judged via an imaging mechanism selected from the group consisting of fMRI, Positron Emission Tomography, and other.

In some variations, the effectiveness of macro-pulse neuromodulation is judged via stimulating motor cortex and assessing the magnitude of motor evoked potentials.

In some variations, the effectiveness of micro-pulse neuromodulation is judged via stimulating motor cortex and assessing the magnitude of motor evoked potentials.

In some variations, the effectiveness of macro-pulse neuromodulation is judged by stimulation the palm and assessing the impact of finger movements.

In some variations, the effectiveness of micro-pulse neuromodulation is judged by stimulation the palm and assessing the impact of finger movements.

In some variations, the Transcranial Magnetic Stimulation pulses rather than ultrasound pulses are shaped.

Thus, disclosed are methods and systems for non-invasive ultrasound stimulation of neural structures, whether the central nervous systems (such as the brain), nerve roots, or peripheral nerves using macro- and micro-pulse shaping. Which macro-pulse and micro-pulse shapes are most effect depends on the target. This can be assessed either by functional results (e.g., doing motor cortex stimulation and seeing which macro- and micro-pulse shape combination causes the greatest motor response) or by imaging (e.g., PET of fMRI) results.

### Summary of Part VIII: Patterned Control of Ultrasound for Neuromodulation

It is one purpose of this invention to provide an ultrasound device delivering enhanced non-invasive superficial or deep-brain neuromodulation using pulse patterns impacting one or a plurality of points in a neural circuit to produce acute effects or Long-Term Potentiation (LTP) or Long-Term Depression (LTD) using up-regulation or down-regulation. Multiple points in a neural circuit can all up regulated, all down regulated or there can be a mixture. Typically LTP is obtained by up-regulation obtained through neuromodulation and LTD obtained by down-regulation obtained through neuromodulation. Two different targets may have different optimal frequency stimulations (even if both up-regulated and down-regulated).

In this invention, this is achieved by individually controlling the pulse pattern applied to each of the ultrasound transducers generating ultrasound beams impacting individual targets. The pulse patterns can be applied to individual ultrasound transducers hitting individual targets or sets of transducers applying ultrasound neuromodulation on a given target using non-intersecting or intersecting ultrasound beams. Pulse patterns can vary in one or both of timing or intensity. Timing patterns may vary either in frequency or inter-pulse or inter-train intervals (e.g., one pulse followed by two pulses with a shorter inter-pulse interval and repeat) for each individual ultrasound transducer.

To assess the efficacy of the patterned neuromodulation, ancillary monitoring or imaging may be employed.

For example, described herein are methods for ultrasound neuromodulation of one or a plurality of deep-brain targets comprising: a. Providing one or a plurality of ultrasound transducers; b. Aiming the beams of said ultrasound transducers at one or a plurality of applicable neural targets; c. modulating the ultrasound transducers with patterned stimulation, whereby the one or a plurality of neural targets are each neuromodulated producing regulation selected from the group consisting of up-regulation and down-regulation.

In some variations, the variation is of one or a plurality selected from the group consisting of inter-pulse intervals and inter-train intervals.

In some variations, the pulse-burst trains are selected from the group consisting of fixed and varied.

In some variations, the inter-pulse-train intervals are selected from the group consisting of fixed and varied.

In some variations, the applied intensity pattern is selected from the group consisting of fixed and varied.

In some variations, the pattern applied is selected from the group consisting of random, theta-burst stimulation.

In some variations, the control system used for control of the patterns is selected from one or a plurality of inputs selected from the group consisting of user input, feedback from imaging system, feedback from functional monitor, and patient input.

In some variations, the relationship among applied frequency pattern, applied timing pattern, and applied intensity pattern is selected from the group consisting of independently varied, dependently varied, independently fixed, and dependently fixed.

In some variations, the pattern is varied during the course of neuromodulation.

In some variations, the effect of patterned ultrasonic neuromodulation is selected from one or more of the group consisting of acute effect, Long-Term Potentiation and Long-Term Depression.

In some variations, the applied pattern is selected from the group of synchronous with all ultrasound transducers using the same pattern and asynchronous with not all ultrasound transducers using the same pattern.

In some variations, the locations of the targets are selected from the group consisting of in the same neural circuit and in different neural circuits.

In some variations, the use of multiple ultrasound transducers is selected from one or a plurality of the group consisting of neuromodulation of the same target and neuromodulation of different targets.

In some variations, the pattern applied in used to avoid side effects elicited by neuromodulation of one or a plurality of structures selected from the group consisting of unintended structures and structures that need to be protected from neuromodulation.

In some variations, the applied pattern is selected from the group of where all targets receive the same pattern and all targets do not receive the same pattern.

In some variations, one set of applied patterns applied to a given neural circuit to provide treatment for one condition and an alternative set of applied patterns is applied to that neural circuit to provide treatment for another condition.

In some variations, one treated condition is the manic phase of bipolar disorder and the other treated condition is the depressive phase of bipolar disorder.

In some variations, the manic phase is treated with neuromodulation causing down-regulation and the depressive phase is treated with neuromodulation causing up-regulation.

Thus, disclosed are methods and devices for ultrasound-mediated non-invasive deep brain neuromodulation impacting one or a plurality of points in a neural circuit using patterned inputs. These are applicable whether the ultrasound beams intersect at the targets or not. Depending on the application, this can produce short-term effects (as in the treatment of post-surgical pain) or long-term effects in terms of Long-Term Potentiation (LTP) or Long-Term Depression (LTD) to treat indications such as neurologic and psychiatric conditions. The ultrasound transducers are used with control of frequency, firing pattern, and intensity to produce up-regulation or down-regulation.

### Summary of Part IX: Ultrasound-Intersecting Beams for Deep-Brain Neuromodulation

It is the purpose of this invention to provide an ultrasound device delivering enhanced non-invasive deep brain or superficial deep-brain neuromodulation impacting one or a plurality of points in a neural circuit to produce acute effects or Long-Term Potentiation (LTP) or Long-Term Depression (LTD) using up-regulation or down-regulation.

For example, described herein are methods for ultrasound neuromodulation of one or a plurality of deep-brain targets comprising: a. attaching a plurality of ultrasound transducers to a positioning frame, and b. aiming the beams from the ultrasound transducers so said beams intersect at the one or plurality of targets, whereby the combination of said ultrasound beams neuromodulates the targeted neural structures producing one or a plurality of regulations selected from the group consisting of up-regulation and down-regulation.

In some variations, the width of the ultrasound transducer and resultant beam are matched to the size of the target.

In some variations, a plurality of ultrasound transducers is employed to neuromodulate multiple targets in multiple neural circuits.

In some variations, one or a plurality of ultrasound transducers is used with control of selected from the group consisting of direction of the energy emission, intensity, frequency (carrier frequency and/or neuromodulation frequency), pulse duration, pulse pattern, and phase/intensity relationships to targeting.

In some variations, one or plurality of targets is up regulated and one or a plurality of targets is down regulated.

In some variations, one or a plurality of targets is hit with a single ultrasound beam.

In some variations, a combination of a plurality of ultrasound transducers and Transcranial Magnetic Stimulation electromagnets is employed to neuromodulate one or a plurality of targets in one or a plurality of neural circuits.

In some variations, ultrasound therapy is combined with or replaced by one of more therapies selected from the group consisting of Transcranial Magnetic Stimulation (TMS), transcranial Direct Current Stimulation (tDCS), Deep-Brain Stimulation (DBS) using implanted electrodes, application of optogenetics, radiosurgery, Radio-Frequency (RF) therapy, behavioral therapy, and medications.

In some variations, the effect is selected from one or more of the group consisting of acute effect, Long-Term Potentiation, Long-Term Depression.

Also described herein are devices for ultrasound neuromodulation of one or a plurality of deep-brain targets comprising: a. attaching a plurality of ultrasound transducers to a positioning frame, and b. aiming the beams from the ultrasound transducers so said beams intersect at the one or plurality of targets, whereby the combination of said ultrasound beams neuromodulates the targeted neural structures producing one or a plurality of regulations selected from the group consisting of up-regulation and down-regulation.

In some variations, the width of the ultrasound transducer and resultant beam are matched to the size of the target.

In some variations, a plurality of ultrasound transducers is employed to neuromodulate multiple targets in multiple neural circuits.

In some variations, one or a plurality of ultrasound transducers is used with control of selected from the group consisting of direction of the energy emission, intensity, frequency (carrier frequency and/or neuromodulation frequency), pulse duration, pulse pattern, and phase/intensity relationships to targeting.

In some variations, one or plurality of targets is up regulated and one or a plurality of targets is down regulated.

In some variations, a plurality of targets is hit with a single ultrasound beam.

In some variations, a combination of a plurality of combination ultrasound transducer and Transcranial Magnetic Stimulation electromagnets is employed to neuromodulate one or a plurality of targets in one or a plurality of neural circuits.

In some variations, ultrasound therapy is combined with or replaced by one of more therapies selected from the group consisting of Transcranial Magnetic Stimulation (TMS), transcranial Direct Current Stimulation (tDCS), Deep-Brain Stimulation (DBS) using implanted electrodes, application of optogenetics, radiosurgery, Radio-Frequency (RF) therapy, behavioral therapy, and medications.

In some variations, the effect is selected from one or more of the group consisting of acute effect, Long-Term Potentiation, Long-Term Depression.

Thus, disclosed are methods and devices for ultrasound-mediated non-invasive deep brain neuromodulation impacting one or a plurality of points in a neural circuit using intersecting ultrasound beams. Depending on the application, this can produce short-term effects (as in the treatment of post-surgical pain) or long-term effects in terms of Long-Term Potentiation (LTP) or Long-Term Depression (LTD) to treat indications such as neurologic and psychiatric conditions. Multiple beams intersect and summate at one or a plurality of targets. The ultrasound transducers are used with control of direction of the energy emission, intensity, frequency (carrier frequency and/or neuromodulation frequency), pulse duration, pulse pattern, and phase/intensity relationships to targeting and accomplishing up-regulation and/or down-regulation.

### Summary of Part X: Ultrasound-Neuromodulation Techniques for Control of Permeability of the Blood-Brain Barrierus

It is the purpose of this invention to provide methods and systems using non-invasive ultrasound-neuromodulation techniques to selectively alter the permeability of the blood-brain barrier (either brain or spinal cord). Early work at Ben-Gurion University and the University of Rome using Brainsway in Transcranial Magnetic Stimulation (TMS) systems has shown that deep-brain neuromodulation techniques can alter the permeability of the blood-brain barrier to allow more effective penetration of drugs (e.g., for the treatment of malignant tumors). Tumors to which opening of the blood-brain barrier using other techniques has been applied are gliomas, CNS lymphoma and metastatic cancer to the brain. The equipment employed in the current invention also costs less and can be portable for use in a variety of settings, including within the home of the patient.

Such neuromodulation can produce acute effects or Long-Term Potentiation (LTP) or Long-Term Depression (LTD). Included is control of direction of the energy emission, intensity, frequency (carrier and/or neuromodulation frequency), pulse duration, firing pattern, and phase/intensity relationships for beam steering and focusing on targets and accomplishing up-regulation and/or down-regulation. Use of ancillary monitoring or imaging to provide feedback is optional. In embodiments where concurrent imaging is performed, the device of the invention is constructed of non-ferrous material.

Multiple targets can be neuromodulated singly or in groups to control the permeability of the blood-brain barrier. To accomplish the treatment, in some cases the neural targets will be up regulated and in some cases down regulated, depending on the given target. The targeting can be done with one or more of known external landmarks, an atlas-based approach or imaging (e.g., fMRI or Positron Emission Tomography).

While ultrasound can be focused down to a diameter on the order of one to a few millimeters (depending on the frequency), whether such a tight focus is required depends on the conformation of the target.

For example, described herein are methods for altering a permeability of a blood-brain barrier in a patient, the method comprising: aiming at least one ultrasound transducer at least one target in a brain or a spinal cord of a human or animal, and energizing at least one transducer to deliver pulsed ultrasound energy to the at least one target, wherein permeability of the blood-brain barrier in the vicinity of the target is altered.

In some variations, the transducer is controlled to deliver ultrasound pulsed power that increases the permeability of the blood-brain barrier.

In some variations, the method further comprises administering a drug to the patient wherein the effectiveness of the drug is enhanced by increased penetration of that drug into the target because of the increase in permeability of the blood-brain barrier.

In some variations, the transducer is controlled to deliver ultrasound pulsed power which decreases the permeability of the blood-brain barrier.

In some variations, the method further comprises administering a drug to the patient wherein the side effects of the drug are reduced due to decreased penetration of the drug into the target because of the decrease in permeability of the blood-brain barrier.

In some variations, a target is selected to have permeability to a drug increased to improve the effectiveness of the drug.

In some variations, a target is selected to have permeability to a drug decreased to protect the target and decrease the side effects of the drug.

In some variations, the ultrasound further provides coincident neuromodulation of a neural target.

In some variations, the neuromodulation comprises up-regulation.

In some variations, the neuromodulation comprises down-regulation.

In some variations, the neuromodulation induces Long-Term Depression.

In some variations, the neuromodulation induces Long-Term Potentiation.

In some variations, aiming comprises aiming a plurality of ultrasonic transducers to produce beams which intersect at a target.

In some variations, said at least one of ultrasound transducers delivers a defocused beam to alter the permeability of large volumes of a target in a brain.

In some variations, the ultrasound energy has a frequency in the range of 0.3 MHz to 0.8 MHz.

In some variations, the ultrasound energy is delivered at a power greater than 20 mW/cm² at a target tissue.

In some variations, the ultrasound energy is delivered at a power less than that causing tissue damage.

In some variations, the ultrasound energy has a stimulation frequency of lower than 500 Hz for inhibition of neural activity.

In some variations, the ultrasound energy has a pulse duration in the range from 0.1 to 20 msec repeated at frequencies of 2 Hz or lower for down regulation.

In some variations, the ultrasound energy has a stimulation frequency for excitation in the range of 500 Hz to 5 MHz.

In some variations, the ultrasound energy has a pulse duration in the range from 0.1 to 20 msec repeated at frequencies higher than 2 Hz for up regulation.

In some variations, the ultrasound has a focus area diameter in the range from 0.5 to 150 mm.

In some variations, the method further comprises applying mechanical perturbations radially or axially to move the ultrasound transducers.

Thus, disclosed are methods and systems and methods employing non-invasive ultrasound-neuromodulation techniques to control the permeability of the blood-brain barrier. For example, such an alteration can permit increased penetration of a medication to increase its therapeutic effect. The neuromodulation can produce acute or long-term effects. The latter occur through Long-Term Depression (LTD) and Long-Term Potentiation (LTP) via training. Included is control of direction of the energy emission, intensity, frequency (carrier and/or neuromodulation frequency), pulse duration, firing pattern, and phase/intensity relationships for beam steering and focusing on targets and accomplishing up-regulation and/or down-regulation.

### Summary of Part XI: Ultrasound Neuromodulation of Spinal Cord

One purpose of this invention to provide methods and systems for neuromodulation of the spinal cord to treat certain types of pain. Such applicable conditions are non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), and refractory ischemic pain (e.g., angina). For pain treatment, the ultrasound energy is targeted to the dorsal column of the spinal cord. In certain embodiments which employ ultrasound neuromodulation, pain is replaced by tingling parathesia. In certain embodiments ultrasound neuromodulation stimulates pain inhibition pathways and can produce acute or long-term effects. The latter can be achieved through long-term potentiation (LTP) or long-term depression (LTD) via training.

The ultrasound energy may be directed at the same target regions in the spinal cord that have been targeted by electrical spinal cord stimulation. For example, for sciatic pain (typically dermatome level L5-S1), ultrasound stimulation can be directed at T10. For angina, the ultrasound energy can be directed at the lower cervical and upper thoracic region. For the abdominal/visceral pain, the ultrasound can be directed at T5-7. Acute and chronic vasculitis can be treated and associated pain by stimulation of regions of the spinal cord as taught in the literature with regard to SCS (Raso, R. and T. Deer, “Spinal Cord Stimulation in the Treatment of Acute and Chronic Vasculitis: Clinical Discussion and Synopsis of the Literature,” Neuromodulation 14:225-228, 2011).

In addition to pain treatment, ultrasound treatment of the spinal cord according to the present invention can treat other conditions such as refractory overactive bladder (e.g., urgency/frequency and urge incontinence) via sacral neuromodulation (Kacker R. and A. K. Das, “Selection of ideal candidates for neuromodulation in refractory overactive bladder,” Current Urology Reports, 11(6):372-378, November 2010) or stimulation of a neurogenic bladder to cause emptying.

Another clinical application of the ultrasound treatments of the present invention comprises the reduction of pain caused by functional bowel disorders such as GI visceral pain and irritable bowel syndrome where myeloperoxidase activity is decreased, inflammation is suppressed, and abdominal relax contractions are inhibited. Suitable target regions in the spinal cord are taught in U.S. Pat. No. 7,251,529.

The present invention further includes control of focus, direction, intensity, frequency (carrier frequency and/or amplitude modulation frequency), pulse duration, pulse pattern, and phase/intensity relationships of the ultrasound energy as well as accomplishing up-regulation and/or down-regulation of the target region of the spinal cord. Use of ancillary monitoring or imaging to provide feedback is optional. In embodiments where concurrent imaging is performed, the device of the invention may be constructed of non-ferrous material.

The specific targets and/or whether the given target is up regulated or down regulated, can depend on the individual patient and relationships of up regulation and down regulation among targets, and the patterns of stimulation applied to the targets. While ultrasound can be focused down to a diameter on the order of one to a few millimeters (depending on the frequency), whether such a tight focus is required depends on the conformation of the neural target.

In a first aspect of the present invention, a method to alleviate a disease condition comprises aiming at least one ultrasound transducer at a target region of a patient's spinal cord. Pulsed power is applied to the transducer to deliver pulsed ultrasound energy to the target region. The disease condition is usually pain where the target region in the spinal cord is typically within the dorsal column. In specific embodiments, the ultrasound transducer is configured to deliver ultrasound energy having an elongated tubular focus aligned with an axis of the spinal cord. Optionally, the ultrasound will be focused where the focus may optionally be mechanically perturbed to enhanced the stimulatory effect of the energy.

In other specific aspects of the methods of the present invention, aiming may comprise aiming a plurality of ultrasonic transducers whose beams intersect at or over the target region. The aiming may alternatively comprise steering a phased array to scan a beam along a segment of the spinal cord. The pulsed ultrasound may provide up-regulation of the target region, e.g. where the ultrasound energy has a modulation frequency of 500 Hz or higher, a pulse duration from 0.1 msec to 20 msec, and a repetition frequency of 2 Hz or higher. Alternatively, the pulsed ultrasound may provide down-regulation of the target region, e.g. where the ultrasound energy has a modulation frequency of 500 Hz or less, a pulse duration from 0.1 msec to 20 msec, and a repetition frequency of 2 Hz or less. In still other specific aspects of the methods of the present invention, the ultrasound energy provides acute, long-term potentiation of the target region. Alternatively, the ultrasound energy may provide acute, long-term depression of the target region. The methods may further comprise the patient providing feedback as well providing a concurrent therapy selected from the group consisting of transcranial magnetic stimulation (TMS), electrical spinal cord stimulation (SCS), and medication.

The pain disease condition being treated may be selected from the group consisting of non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), refractory pain due to ischemia (e.g. angina), acute vasculitis, chronic vasculitis, hyperactive bladder, and neurogenic bladder.

Dorsal lateral lower motor neurons are associated with the lateral corticospinal tract. Ventromedial lower motor neurons are associated with the anterior corticospinal tract. In an embodiment of the current invention, ultrasound neuromodulation exciting of those motor neurons or their associated tracts results in contractions of the connected muscles. Thus in some embodiments, the ultrasound energy can be employed to restore motor neuron function.

In a second aspect of the present invention, apparatus for delivering ultrasound energy to a target region of a patient's spinal cord comprises an ultrasound transducer assembly and control circuitry and/or supporting structure for delivering ultrasound energy from the transducer assembly to the target region of the spinal cord. The ultrasound energy delivery control circuitry and/or supporting structure preferably focuses the ultrasound along a tubular target region aligned with an axis of the spinal cord. The transducer may comprise an elongated transducer having an active surface formed over a partial tubular groove for focusing the ultrasound energy along the tubular target region. The transducer body may consist of a single piezoelectric element or alternatively may include an array of individual transducer elements, e.g. arranged as a phased array for focusing the energy in the tubular focus or other desired focus geometry. The ultrasound transducer may be supported or controlled to mechanically perturb the ultrasound energy, e.g. the ultrasound transducers may be moved to apply mechanical perturbations radially and/or axially. In specifically preferred aspects, the ultrasound transducer and the energy delivery means may be configured to deliver ultrasound energy to the patient's dorsal column for the treatment of pain.

In still other aspects of the present invention, the ultrasound transducer and the energy delivery structure may be configured to deliver ultrasound energy to up-regulate or down-regulate the target region. The ultrasound transducer and the energy delivery control and support structure may be configured to deliver ultrasound energy with a modulation frequency of 500 Hz or less, a pulse duration from 0.1 msec to 20 msec, and a repetition frequency of 2 Hz or less to down regulate the target region. Alternatively the ultrasound transducer and the energy delivery control and support structure may be configured to deliver ultrasound energy with a modulation frequency of 500 Hz or higher, a pulse duration from 0.1 msec to 20 msec, and a repetition frequency of 2 Hz or higher to up regulate the target region.

Apparatus of the present invention may be further configured to deliver ultrasound energy that provides long-term potentiation of the target region long-term depression of the target region. Apparatus may further comprise a patient feedback mechanism and may further be combined with system elements for delivering transcranial magnetic stimulation (TMS), electrical spinal cord stimulation (SCS).

For example, described herein are methods to alleviate a disease condition, the method comprising: aiming at least one ultrasound transducer at a target region of a patient's spinal cord, and applying pulsed power to the transducer to deliver pulsed ultrasound energy to the target region.

In some variations, the disease condition is pain and the target region comprises the dorsal column.

In some variations, the ultrasound transducer is configured to deliver ultrasound energy having an elongated tubular focus aligned with an axis of the spinal cord.

In some variations, the method further comprises mechanically perturbing the ultrasound energy.

In some variations, aiming comprises aiming a plurality of ultrasonic transducers whose beams intersect at or over the target region.

In some variations, aiming comprises steering an ultrasound beam from a phased ultrasound array.

In some variations, the pulsed ultrasound provides up-regulation of the target region.

In some variations, the ultrasound energy has a modulation frequency of 500 Hz or higher, a pulse duration from 0.1 msec to 20 msec, and a repetition frequency of 2 Hz or higher.

In some variations, the pulsed ultrasound provides down-regulation of the target region.

In some variations, the ultrasound energy has a modulation frequency of 500 Hz or less, a pulse duration from 0.1 msec to 20 msec, and a repetition frequency of 2 Hz or less.

In some variations, ultrasound energy provides acute, long-term potentiation of the target region.

In some variations, ultrasound energy provides acute, long-term depression of the target region.

In some variations, the disease treated is selected from the group consisting of non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), refractory pain due to ischemia (e.g. angina), acute vasculitis, chronic vasculitis, hyperactive bladder, and neurogenic bladder.

In some variations, the pulsed ultrasound energy produces motor neurons.

In some variations, the method further comprises the patient providing feedback.

In some variations, the method further comprises providing a concurrent therapy selected from the group consisting of transcranial magnetic stimulation (TMS), electrical spinal cord stimulation (SCS), and medication.

Also described herein are Apparatuses for delivering ultrasound energy to a target region of a patient's spinal cord, said apparatus comprising: an ultrasound transducer assembly, and means for delivering ultrasound energy from the transducer assembly to the target region of the spinal cord.

In some variations, the ultrasound energy deliver means focuses the ultrasound along a tubular target region aligned with an axis of the spinal cord.

In some variations, the transducer comprises an elongated transducer having an active surface formed over a partial tubular groove for focusing the ultrasound energy along the tubular target region.

In some variations, the transducer body consists of a single piezoelectric element.

In some variations, the transducer comprises a phased array having a length and width which configure to a segment of a spinal cord.

In some variations, the means for delivering ultrasound energy from the transducer assembly to the target region of the spinal cord is configured to mechanically perturb the ultrasound energy.

In some variations, the ultrasound transducers are moved to apply mechanical perturbations radially and/or axially.

In some variations, the ultrasound transducer and the energy delivery means are configured to deliver ultrasound energy to the patient's dorsal column for the treatment of pain.

In some variations, the ultrasound transducer and the energy delivery means are configured to deliver ultrasound energy to up-regulate the target region.

In some variations, the ultrasound transducer and the energy delivery means are configured to deliver ultrasound energy to down-regulate the target region.

In some variations, the ultrasound transducer and the energy delivery means are configured to deliver ultrasound energy with a modulation frequency of 500 Hz or less, a pulse duration from 0.1 msec to 20 msec, and a repetition frequency of 2 Hz or less to down regulate the target region.

In some variations, the ultrasound transducer and the energy delivery means are configured to deliver ultrasound energy with a modulation frequency of 500 Hz or higher, a pulse duration from 0.1 msec to 20 msec, and a repetition frequency of 2 Hz or higher to up regulate the target region.

In some variations, the ultrasound transducer and the energy delivery means are configured to deliver ultrasound energy which provides long-term potentiation of the target region.

In some variations, the ultrasound transducer and the energy delivery means are configured to deliver ultrasound energy which provides long-term depression of the target region.

In some variations, the apparatus further comprises a patient feedback mechanism.

In some variations, the apparatus further comprises a means for delivering transcranial magnetic stimulation (TMS) or electrical spinal cord stimulation (SCS).

Thus, described are methods and systems for non-invasive neuromodulation of the spinal cord utilize a transducer to deliver pulsed ultrasound energy to up regulate or down regulate neural targets for the treatment of pain and other disease conditions. The systems provide control of direction of the energy emission, intensity, frequency, pulse duration, pulse pattern, mechanical perturbation, and phase/intensity relationships to achieve up regulation and/or down regulation. One embodiment focuses an elongate tubular ultrasound beam which can be aligned with a target region of the spinal cord.

### Summary of Part XII: Ultrasound Neuromodulation for Diagnosis and Other-Modality Preplanning

The embodiments described herein provide improved methods and systems for patient diagnosis or patient treatment planning. The systems and methods may provide non-invasive neuromodulation using ultrasound for diagnosis or treatment of the patient. The systems and methods can be well suited for diagnosing one or more conditions of the patient from among a plurality of possible conditions having one or more similar symptoms. The treatment planning may comprise pre-treatment planning based on ultrasonic assessment with focused ultrasonic pulses directed to one or more target locations of the patient. Based on the evaluation of symptoms or other outcomes in response to targeting a location with ultrasound, the patient treatment at the target location can be confirmed before the patient is treated.

In a first aspect, embodiments provide a method of neuromodulation of a patient. A pulsed ultrasound is provided to one or more neural targets. A neural disorder is identified or treatment is planned for the neural disorder based on a response of the one or more neural targets to the pulsed ultrasound.

In another aspect, embodiments provide a system for neuromodulation. The system comprises circuitry coupled to one or more ultrasound transducers to provide pulsed ultrasound to one or more neural targets. A processor is coupled to the circuitry. The processor is configured to identify a neural disorder or plan for treatment of the neural disorder based on a response of the one or more neural targets to the pulsed ultrasound.

The ultrasound pulses as described herein can be used in many ways. The pulses can be used at one or more sessions to diagnose the patient, confirm subsequent treatment, or treat the patient, and combinations thereof. The pulses can be shaped in one or more ways, and can be shaped with macro pulse shaping, amplitude modulation of the pulses, and combinations thereof, for example.

In many embodiments, the amplitude modulation frequency of lower than 500 Hz is applied for inhibition of neural activity. The amplitude modulation frequency of lower than 500 Hz can be divided into pulses 0.1 to 20 msec. repeated at frequencies of 2 Hz or lower for down regulation. The amplitude modulation frequency for excitation can be in the range of 500 Hz to 5 MHz. The amplitude modulation frequency of 500 Hz or higher may be divided into pulses 0.1 to 20 msec. repeated at frequencies higher than 2 Hz for up regulation.

In many embodiments, the spinal cord can be treated. Target regions in the spinal cord which can be treated using the ultrasound neuromodulation protocols of the present invention comprise the same locations targeted by electrical SCS electrodes for the same conditions being treated, e.g., a lower cervical-upper thoracic target region for angina, a T5-7 target region for abdominal/visceral pain, and a T10 target region for sciatic pain. Ultrasound neuromodulation in accordance with the present invention can stimulate pain inhibition pathways that in turn can produce acute and/or long-term effects. Other clinical applications of ultrasound neuromodulation of the spinal cord include non-invasive assessment of neuromodulation at a particular target region in a patient's spinal cord prior to implanting an electrode for electrical spinal cord stimulation for pain or other conditions.

In many embodiments the ultrasound neuromodulation of the target may include non-invasive assessment of neuromodulation at a particular target neural region in a patient prior to implanting an electrode for electrical stimulation for pain or other conditions as described herein.

In many embodiments, the feasibility of using Deep Brain Stimulation (DBS) is determined for treatment of depression and to test whether depression symptoms can be mitigated with stimulation of the Cingulate Genu. Dramatic results may occur in some patients (e.g., description as having “lifted the void”). Such results, however, may not occur, so neuromodulation of the Cingulate Genu with ultrasound and determining the patient's response can identify those who would benefit from DBS of that target so as to confirm treatment of the Cingulate Genu target.

In many embodiments, the target site for DBS for the treatment of motor symptoms (e.g., bradykinesia, stiffness, tremor) of Parkinson's Disease (PD) comprises the Subthalamic Nucleus (STN). Stimulation of the STN may well have side effects (e.g., problems with speech, swallowing, weakness, cramping, double vision) because sensitive structures are close to it. An alternative target for the treatment of Parkinson's Disease is the Globus Pallidus interna (GPi) which can be effective in motor symptoms as well as dystonia (e.g., posturing and painful cramping). Which of these two targets will overall be best for a given patient depends on that patient and can be determined based on the patient response to DBS. Stimulation of either the GPi or STN improves many features of advanced PD, and even though STN stimulation can be effective, stimulation of the GPi can be an appropriate DBS target to determine whether the STN or GPi should be treated.

In many embodiments, the target comprises the Ventral Intermediate Nucleus of the Thalamus (Vim), which is related to motor symptoms such as essential tremor. In some embodiments, patients with tremor as their dominant symptom benefit from Vim stimulation even though other symptoms are not ameliorated, since such stimulation can deliver the best “motor result.”

In many embodiments, DBS is used on both the STN and the Vim on the same side, such that a plurality of target sites is confirmed and treated.

In many embodiments, ultrasound neuromodulation is used to select the best target for the given patient with the given condition based on testing the results of stimulating different targets. DBS stimulation of each of the potential Parkinson's Disease targets may elicit side effects that are patient specific, for example targets comprising one or more of STN, GPi, or Vim. Alternatively or in combination, ultrasound neuromodulation of the spinal cord can be used to assess whether pain has been relieved and to evaluate the potential effectiveness of or parameters for Spinal Cord Stimulation (SCS) using invasive electrode stimulation.

In many embodiments related to diagnosis and preplanning, patient feedback can be used to adjust ultrasound neuromodulation parameters for at least some conditions as described herein. In some embodiments, ultrasound neuromodulation can be used to retrain neural pathways over time, such that the patient can be treated without constant stimulation of DBS.

Alternatively or in combination with preplanning, ultrasound neuromodulation can be used to diagnosis the patient. In many embodiments, an accurate diagnosis may be difficult with prior methods and apparatus because of the way the disorder manifests itself. In many embodiments, diagnostic the methods and apparatus as described herein provide differentiation between the tremor of Parkinson's Disease and essential tremor. In many embodiments, the tremor of Parkinson's Disease typically occurs at rest and essential tremor does not or is accentuated by movement. An area of confusion is that some patients with Parkinson's Disease have tremor at rest as well.

The methods and apparatus as described herein provide a higher probability of getting the correct diagnosis and can differentiate between essential tremor and the tremor of Parkinson's Disease, such that the patient can be provided with proper treatment. The drug treatments are different for Parkinson's disease and essential tremor. The treatment of Parkinson's Disease in accordance with embodiments comprises treatment with one or more of levodopa, dopamine agonists, MAO-B inhibitors, and other drugs such as amantadine and anticholinergics. The treatment of essential tremor comprises one or more of beta blockers, propranolol, antiepileptic agents, primidone, or gabapentin. The higher probability of getting the right diagnosis can be beneficial with respect to drug treatment in a number of people with essential tremor who may also suffer fear of public situations. In at least some embodiments, medicines used to treat essential tremor may also increase a person's risk of becoming depressed. Embodiments as described herein can improve surgical treatments, as pallidotomy or thalamotomy can be used for either Parkinson's Disease or essential tremor but pallidotomy is generally not effective for essential tremor. The diagnostic methods and apparatus can differentiate between Parkinson's disease and essential tremor, for example when imaging by one or more of CT or MRI scans is insufficient to make a diagnosis. Many embodiments provide the ability to allow the correct selection of therapies selected from among one or more of surgical, neuromodulation, or drug therapies.

While ultrasound neuromodulation can produce acute effects or Long-Term Potentiation (LTP) or Long-Term Depression (LTD), the acute effects are used in many embodiments as described herein. The embodiments as described herein provide control of direction of the energy emission, intensity, frequency (carrier frequency and/or neuromodulation frequency), pulse duration, pulse pattern, and phase/intensity relationships to targeting and accomplishing up-regulation and/or down-regulation. Ancillary monitoring or imaging to provide feedback can be optionally and beneficially combined with the ultrasonic systems and methods as described herein. In many embodiments where concurrent imaging is performed, such as MRI imaging, the systems and methods may comprise non-ferrous material.

In many embodiments, single or multiple targets in groups can be neuromodulated to evaluate the feasibility of treatment and to preplan treatment using neuromodulation modalities, which may comprise non-ultrasonic or ultrasonic modalities, for example. To accomplish this evaluation, in some embodiments the neural targets will be up regulated and in some embodiments down regulated, and combinations thereof, depending on the identified neural target under evaluation. In many embodiments, the targets can be identified by one or more of PET imaging, fMRI imaging, clinical response to Deep-Brain Stimulation (DBS), or Transcranial Magnetic Stimulation (TMS).

In many embodiments, the identified targets depend on the patient and the relationships among the targets of the patient. In some embodiments, multiple neuromodulation targets will be bilateral and in other embodiments ipsilateral or contralateral. The specific targets identified and/or whether the given target is up regulated or down regulated, can depend upon the individual patient and the relationships of up regulation and down regulation among targets, and the patterns of stimulation applied to the targets identified for the patient.

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and in terms of the cost of administering the therapy.

While ultrasound can be focused down to a diameter on the order of one to a few millimeters (depending on the frequency), whether such a tight focus is required depends on the configuration of the neural target. In order to determine feasibility or preplan treatment by an invasive neuromodulation modality a non-invasive mechanism must be used. Among non-invasive methods, ultrasound neuromodulation is more focused than Transcranial Magnetic Stimulation so it inherently offers more capability to demonstrate the feasibility of and preplan treatment planning for invasive and in many cases highly focused neuromodulation modalities such as Deep-Brain Stimulation (DBS).

For example, described herein are methods of neuromodulation of a patient, the method comprising: providing pulsed ultrasound to one or more neural targets of a neural disorder; and identifying the neural disorder or planning for treatment of the neural disorder based on a response of the one or more neural targets to the pulsed ultrasound.

In some variations, planning for treatment of the neural disorder comprises determining parameters of the pulsed ultrasound in order to confirm a neuromodulation therapy in order to treat the neural disorder based on a response of the one or more neural targets to the parameters.

In some variations, planning for treatment comprises preplanning for a neuromodulation therapy comprising one or more of surgical, invasive neuromodulation, non-invasive neuromodulation, behavioral therapy, or drugs.

In some variations, patient feedback is used to adjust symptoms selected from the group of pain, depression, tremor, voiding from neurogenic bladder; and wherein the symptoms are adjusted based on the one or more neural targets and parameters of the pulsed ultrasound.

In some variations, the identifying the neural disorder comprising differentiating between the tremor of Parkinson's Disease and essential tremor.

In some variations, the planning for treatment comprises identifying a response to neuromodulation of the Cingulate Genu for the purpose of treating depression.

In some variations, planning for treatment comprises identifying a response to neuromodulation of the spinal cord for the purpose of reducing pain.

In some variations, the one or more targets are neuromodulated in a manner selected from the group consisting of ipsilateral neurmodulation, contralateral neuromodulation, and bilateral neuromodulation.

In some variations, one or more energy sources is used to treat the neural disorder, the one or more energy sources selected from the group consisting of Transcranial Magnetic Stimulation (TMS) and transcranial Direct Current Stimulation (tDCS).

In some variations, a feedback mechanism is applied, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, and a subjective patient response.

Also described herein are systems for neuromodulation, the system comprising: circuitry coupled to one or more ultrasound transducers to provide pulsed ultrasound to one or more neural targets; a processor coupled to the circuitry, the processor configured to identify a neural disorder or plan for treatment of the neural disorder based on a response of the one or more neural targets to the pulsed ultrasound.

In some variations, the processor comprises instructions to plan for treatment of the neural disorder, including determining parameters of the pulsed ultrasound in order to confirm a neuromodulation therapy in order to treat the neural disorder based on a response of the one or more neural targets to the parameters.

In some variations, the processor comprises instructions to plan for treatment, including preplanning for a neuromodulation therapy comprising one or more of surgical, invasive neuromodulation, non-invasive neuromodulation, behavioral therapy, or drugs.

In some variations, the processor comprises instructions to receive patient feedback in order to adjust symptoms selected from the group of pain, depression, tremor, voiding from neurogenic bladder; and wherein the symptoms are adjusted based on the one or more neural targets and parameters of the pulsed ultrasound.

In some variations, the processor comprises instructions to identify the neural disorder comprising differentiating between the tremor of Parkinson's Disease and essential tremor.

In some variations, the processor comprises instructions to plan for treatment, including identifying a response to neuromodulation of the Cingulate Genu for the purpose of treating depression.

In some variations, the processor comprises instructions to plan for treatment, including identifying a response to neuromodulation of the spinal cord for the purpose of reducing pain.

In some variations, the processor comprises instructions to neuromodulate the one or more targets in a manner selected from the group consisting of ipsilateral neurmodulation, contralateral neuromodulation, and bilateral neuromodulation.

In some variations, the processor comprises instruction to preplan for treatment based on one or more energy sources which is used to treat the neural disorder, the one or more energy sources selected from the group consisting of Transcranial Magnetic Stimulation (TMS) and transcranial Direct Current Stimulation (tDCS).

In some variations, the processor system comprises instructions of an applied feedback mechanism, wherein the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, and a subjective patient response.

In some variations, the processor system comprises instructions to pre-plan for treatment of the neural disorder and wherein the neural disorder comprises one or more of depression, Parkinson's disease, essential tremor, bipolar disorder or spinal cord pain and wherein the target site evaluated prior to treatment comprises one or more of a Cingulate Genu, DBS, STN, GPi, Vim, Nucleus accumbens, Area 25 of subcallosal cingulate, one or more levels of a spinal column, white matter or ganglia.

In some variations, the processor system comprises instructions to diagnose the neural disorder and wherein a symptom of the neural disorder comprises one or more of depression, tremor, bipolar behavior or pain and wherein the target site evaluated comprises one or more of Cingulate Genu, DBS, STN, GPi, Vim, Nucleus accumbens, area of 25 of subcallosal cingulate, one or more levels of the spinal column, whiter matter or ganglia.

Thus, disclosed are methods and systems for non-invasive neuromodulation using ultrasound for diagnosis to evaluate the feasibility of and preplan neuromodulation treatment using other modalities. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, mechanical perturbation, and phase/intensity relationships to targeting and accomplishing up regulation and/or down regulation.

### Summary of Part XIII: Planning and Using Sessions of Ultrasound for Neuromodulation

Also disclosed are systems and methods for non-invasive neuromodulation using ultrasound delivered in sessions. Examples of session types include periodic over extended time, periodic over compressed time, and continuous. Maintenance sessions are either periodic maintenance sessions or as-needed maintenance tune-up sessions. The neuromodulation can produce acute or long-term effects. The latter occur through Long-Term Depression (LTD) and Long-Term Potentiation (LTP) via training. Included is control of direction of the energy emission, intensity, frequency, pulse duration, pulse pattern, and phase/intensity relationships to targeting and accomplishing up regulation and/or down regulation.

It is the purpose of some variations of the inventions described herein to provide methods and systems for non-invasive neuromodulation using ultrasound delivered in sessions. This is important because different conditions and patients need different treatment regimens. Examples of session types include periodic over extended time, periodic over compressed time, and continuous. Periodic sessions over extended time typically means a single session of length on the order of 30 to 60 minutes repeated daily or five days per week over a four to six weeks. Other lengths of session or number of weeks of neuromodulation are applicable, such as session lengths up to 2.5 hours and number of weeks ranging from one to eight. Period sessions over compressed time typically means a single session of length on the order of 30 to 60 minutes repeated during awake hours with inter-session times of 30 minutes to 60 minutes over one to two days. Other inter-session times such as 15 minutes to three hours and days of compressed therapy such as one to five days are applicable.

In addition, considerations include both periodic maintenance sessions and/or as-needed maintenance tune-up sessions. Maintenance categories are Maintenance Post Completion of Original Treatment at Fixed Intervals and Maintenance Post Completion of Original Treatment with As-Needed Maintenance Tune-Ups. An example of the former are with one or more 50-minutes sessions during week 2 of months four and eight, and of the latter is one or more 50-minute sessions during week 7 because a tune up is needed at that time as indicated by return of symptoms. Sessions using ultrasound neuromodulation are not just applicable to deep-brain neuromodulation. Size and cost of the ultrasound neuromodulation equipment in many circumstances may make it impractical to deliver the energy continuously. An example of an exception is the case where patient being treated is comatose and the energy can be delivered continuously. Another example is the control of hypertension during a hypertensive crisis and the patient cooperates by remaining relative stationary. Of course, for configurations (e.g., superficial targets) requiring less power and fewer ultrasound transducers, ambulatory use is practical (continuous neuromodulation or otherwise). Ultrasound neuromodulation can produce acute effects or Long-Term Potentiation (LTP) or Long-Term Depression (LTD). Included is control of direction of the energy emission, intensity, frequency (carrier frequency and/or neuromodulation frequency), pulse duration, pulse pattern, and phase/intensity relationships to targeting and accomplishing up-regulation and/or down-regulation. Use of ancillary monitoring or imaging to provide feedback is optional. In embodiments where concurrent imaging is performed, the device of the invention is constructed of non-ferrous material.

Sessions can be applied to the following conditions, but not limited to them: Depression and Bipolar Disorder, pain, addiction, tinnitus, motor disorders, epilepsy, stroke, Reticular Activating System, Traumatic Brain Injury & Concussion, Tourette's Syndrome, Alzheimer's Disease, Anxiety Disorder, Obsessive Compulsive Disorder, Cognitive Enhancement, Autism, Obesity, Eating Disorders, Attention Deficit Hyperactivity Disorder, Post-Traumatic Stress Disorder, Schizophrenia, GI Motility, Orgasmatron, Compulsive Sexual Behavior, Spheno-Palatine Ganglion, Occiput, and Spinal Cord Stimulation.

Any target is applicable. Multiple targets can be neuromodulated singly or in groups. To accomplish the treatment, in some cases the neural targets will be up regulated and in some cases down regulated, depending on the given neural target. Targets have been identified by such methods as PET imaging, fMRI imaging, and clinical response to Deep-Brain Stimulation (DBS) or Transcranial Magnetic Stimulation (TMS). Targets depend on specific patients and relationships among the targets. In some cases neuromodulation will be bilateral and in others unilateral. The specific targets and/or whether the given target is up regulated or down regulated, can depend on the individual patient and relationships of up regulation and down regulation among targets, and the patterns of stimulation applied to the targets. The effectiveness of the neuromodulation will depend on session characteristics in terms of how frequently and how long the neuromodulation is applied.

Transcranial Magnetic Stimulation is typically delivered in the periodic over extended time mode (e.g., the Neuronetics recommended protocol is 5 days per week, 40 to 50 minutes per day, for six weeks). There are studies underway for accelerated treatment (periodic over compressed time). An example is the Veteran's Administration Trial (clinicaltrials.gov ID NCT00248768) whose purpose is to determinate if accelerated rTMS (repetitive Transcranial Magnetic Stimulation) treatment over 1.5 days is effective for ameliorating depression in Parkinson's disease. The rTMS Treatments consist of 1000 total pulses at 10 Hz and 100% motor threshold administered hourly for 1.5 days, totaling 15 sessions. Of course, 1.5 days is significantly shorter than four to six weeks. Positive results for the trial were reported (Holtzheimer P E 3rd, McDonald W M, Mufti M, Kelley M E, Quinn S, Corso G, and C M Epstein, “Accelerated repetitive transcranial magnetic stimulation for treatment-resistant depression,” Depress Anxiety. 2010 October; 27(10):960-3). Continuous stimulation is not practical with TMS because of the cost and size of the equipment required. As to maintenance therapy, approaches vary, but post-maintenance can range from periodic (even beginning short term like once per week beginning just after the end of the initial treatment) to on an as-needed basis (e.g., can involve two to 10 treatments delivered when symptoms return (e.g., 6 months to two years after initial treatment)).

The targeting can be done with one or more of known external landmarks, an atlas-based approach or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target.

For example, described herein are methods of deep-brain neuromodulation using ultrasound stimulation, the method comprising: aiming one or a plurality of ultrasound transducer at one or a plurality of neural targets related to the condition being treated, and applying pulsed power to the ultrasound transducer via a control circuit, whereby the ultrasound neuromodulation is delivered in sessions.

In some variations, the length of session is between 15 minutes and two and a half hours.

In some variations, the type of session is selected from the group consisting of periodic over extended time, periodic over compressed time, and continuous.

In some variations, the extended time involves daily sessions daily or five days per week over a period of one to six weeks.

In some variations, the compressed time is one to five days.

In some variations, the compressed time included inter-session time between 15 minutes to three hours.

In some variations, the maintenance mode is selected from the group consisting of maintenance post-completion of original treatment at fixed intervals and maintenance post-completion of original treatment with as-needed maintenance tune-ups.

The method may further comprise aiming an ultrasound transducer neuromodulating neural targets in a manner selected from the group of up-regulation, down-regulation.

In some variations, the effect is chosen from the group consisting of acute, Long-Term Potentiation, and Long-Term Depression.

In some variations, sessions are applied for the treatment of Depression and Bipolar Disorder.

In some variations, ultrasonic-transducer neuromodulation is targeted to one or a plurality targets selected from the group consisting of the Orbito-Frontal Cortex (OFC), Anterior Cingulate Cortex (ACC), and Insula.

In some variations, sessions are applied to one or more conditions selected from the group consisting of but not limited to Depression and Bipolar Disorder, pain, addiction, tinnitus, motor disorders, epilepsy, stroke, Reticular Activating System, Traumatic Brain Injury & Concussion, Tourette's Syndrome, Alzheimer's Disease, Anxiety Disorder, Obsessive Compulsive Disorder, Cognitive Enhancement, Autism, Obesity, Eating Disorders, Attention Deficit Hyperactivity Disorder, Post-Traumatic Stress Disorder, Schizophrenia, GI Motility, Orgasmatron, Compulsive Sexual Behavior, Spheno-Palatine Ganglion, Occiput, and Spinal Cord Stimulation.

In some variations, a single ultrasonic transducer aimed at a given target is replaced by a plurality of ultrasonic transducers whose beams intersect at that target.

In some variations, a feedback mechanism is applied, where the feedback mechanism is selected from the group consisting of functional Magnetic Resonance Imaging (fMRI), Positive Emission Tomography (PET) imaging, video-electroencephalogram (V-EEG), acoustic monitoring, thermal monitoring, patient.

In some variations, ultrasound therapy is combined with or replaced by one or more therapies selected from the group consisting of Transcranial Magnetic Stimulation (TMS), deep-brain stimulation (DBS), application of optogenetics, radiosurgery, Radio-Frequency (RF) therapy, behavioral therapy, and medications.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the characteristics of the various neuromodulation modalities.

FIG. 2 is a table of Indications versus Targets.

FIG. 3 shows a table for Therapeutic-Modality Combinations for Selected Indications.

FIG. 4 shows the physical layout of the combination of therapeutic modalities for the treatment of pain.

FIG. 5 shows the physical layout of the combination of therapeutic modalities for the treatment of depression.

FIG. 6 shows the physical layout of the combination of therapeutic modalities for the treatment of addiction.

FIG. 7 shows the physical layout of the combination of therapeutic modalities for the treatment of obesity.

FIG. 8 shows the physical layout of the combination of therapeutic modalities for the treatment of epilepsy.

FIG. 9 shows a block diagram of the treatment planning and control system.

FIG. 10 illustrates the flow of the treatment planning and control system.

FIGS. 11A-11C show top and frontal views of the track around the head on which transducers run.

FIGS. 12A-12C illustrate the frontal and side views of an example of the transducer with its hemispheric ultrasound array.

FIG. 13 shows an alternative embodiment in which the transducer is rotated while it is going around the track.

FIG. 14 illustrates an embodiment in which the apparatus is enclosed within a shell.

FIG. 15 shows a block diagram of the control circuit.

FIG. 16 illustrates a simplified neural circuit for addiction.

FIG. 17 illustrates targeting multiple targets in a neural circuit for addiction.

FIG. 18 demonstrates using a patient-specific holder to fix the transducers relative to the target.

FIG. 19 shows an embodiment where the transducers can be moved in and out for patient-specific targeting.

FIG. 20 shows a control mechanism in which the patient controls delivery parameters to optimize delivery impact.

FIG. 21 illustrates a set of neural targets that are to be down-regulated using ultrasound neuromodulation under patient-feedback control to adjust acute pain.

FIG. 22 shows a block diagram of the feedback control algorithm.

FIGS. 23A-23B shows an ultrasound transducer array configured to produce an elongated pencil-shaped focused field.

FIG. 24 illustrates the elongated ultrasound transducer array with sound conduction medium.

FIG. 25 illustrates the neural-circuit diagram for addiction.

FIG. 26 shows physical target layout for addiction.

FIGS. 27A-27C demonstrate two ultrasound transducer arrays with different radii.

FIGS. 28A-28C demonstrate flat transducer array with interchangeable lenses.

FIGS. 29A-29B show a linear ultrasound phased array with steered-beam linearly moving field.

FIGS. 30A-30B demonstrates the combination of ultrasound transducer with TMS Coil.

FIG. 31 shows a control block diagram.

FIG. 32 shows a block diagram of the treatment planning

FIG. 33 illustrates a configuration of exemplar deep-brain targets.

FIG. 34 shows a diagram of a treatment plan with an ultrasound configuration mapped onto the target configuration.

FIG. 35 illustrates the treatment-planning algorithm.

FIG. 36 shows ultrasound transducers and EMG sensors at various portions of the nervous system.

FIGS. 37A-37D show a diagram of the ultrasound sensor, ultrasound conduction medium, ultrasound field, and the target.

FIG. 38 shows a block diagram of the control circuit.

FIGS. 39A-39D show diagrams of macro-pulse shaping.

FIGS. 40A-40C show diagrams of micro-pulse shaping.

FIG. 41 shows a block diagram of the system for generating the output incorporating macro- and micro-pulse shaping.

FIGS. 42A-42F illustrate a table of neuromodulation patterns.

FIG. 43 shows a block diagram of neural circuit in the brain for addiction.

FIG. 44 illustrates four ultrasound transducers targeting four targets in the neural addiction circuit including the Orbito-Frontal Cortex (OFC), the Dorsal Anterior Cingulate Gyms (DACG), the Insula, and the Nucleus Accumbens.

FIG. 45 illustrates the neural circuit allowing alternative effects depending on whether the circuit is up regulated or down regulated.

FIG. 46 shows a block diagram of the mechanism for controlling the multiple ultrasound beams.

FIG. 47 shows a flat ultrasound transducer producing a parallel beam.

FIG. 48 shows three flat ultrasound transducers using global ultrasound conduction medium with beams intersecting on a Dorsal Anterior Cingulate Gyms (DACG) target.

FIG. 49 shows three flat ultrasound transducers using individual ultrasound conduction media with beams intersecting on a Dorsal Anterior Cingulate Gyms (DACG) target.

FIG. 50 shows two sets of flat ultrasound transducers using global ultrasound conduction medium with beams intersecting on Dorsal Anterior Cingulate Gyms (DACG) and Insula targets.

FIG. 51 shows a block diagram of the mechanism for controlling the multiple ultrasound beams.

FIG. 52 shows exemplar blood-brain barrier targets on which ultrasound is focused.

FIG. 53 shows a block diagram of the control circuit.

FIG. 54 shows ultrasound-transducer targeting of the spinal cord from the perspective view of the spinal column.

FIG. 55 shows ultrasound-transducer targeting of the spinal cord from the cross-section view of the spinal column.

FIGS. 56A-56C illustrate shaping of the ultrasound field.

FIGS. 57A and 57B show the mechanism for mechanical perturbation and examples the resultant ultrasound field shapes.

FIG. 58 shows a block diagram of the control circuit.

FIG. 59 illustrates a block diagram for a mechanism providing patient feedback for adjustment of the characteristics of the neuromodulation.

FIG. 60 shows ultrasound-transducer targeting of the STN and the GPi to test the feasibility of using DBS for treatment of Parkinson's Disease, in accordance with embodiments;

FIG. 61 shows targeting of the Cingulate Genu to test the feasibility of using DBS for the treatment of Depression, in accordance with embodiments;

FIG. 62 demonstrates ultrasound neuromodulation of the spinal cord to test the feasibility of using Spinal-Cord Stimulation (SCS) for the treatment of neuropathic or ischemic pain, in accordance with embodiments;

FIGS. 63A and 63B show the mechanism for mechanical perturbation and examples the resultant ultrasound field shapes, in accordance with embodiments;

FIG. 64 shows a block diagram of the control circuit, in accordance with embodiments;

FIG. 65 shows a block diagram of feedback control circuit, in accordance with embodiments;

FIG. 66 illustrates a method and steps for pre-planning, in accordance with embodiments;

FIG. 67 illustrates a method and steps for diagnosis, in accordance with embodiments; and

FIG. 68 shows an apparatus to one or more of diagnose or treat the patient, in accordance with embodiments.

FIGS. 69A-69E show a diagram of exemplar session types for both initial treatment and maintenance sessions.

FIG. 70 shows ultrasonic-transducer targeting of the Orbito-Frontal Cortex (OFC), Anterior Cingulate Cortex (ACC), and Insula for the treatment of depression and bipolar disorder.

FIG. 71 shows a block diagram of the control circuit.

### DETAILED DESCRIPTION

Described herein are methods, systems, and devices of neuromodulation. Each of the twelve sections below describes different aspects, devices, methods, and systems directed to neuromodulation and associated techniques. References to “the invention” may refer to one of the various inventions described herein; elements of one inventions need not be incorporated or necessary for other inventions.

### Part I: Multi-Modality Neuromodulation of Brain Targets

It is the purpose of some of the inventions described to provide methods and systems and methods for deep brain or superficial stimulation using multiple therapeutic modalities to impact one or multiple points in a neural circuit to produce Long-Term Potentiation (LTP) or Long-Term Depression (LTD). Some of the modalities (e.g., TMS) will cause training or retraining to bring about long-term change. Radiosurgery (or a surgical ablation) on the other hand will cause a permanent effect and DBS must remain applied or the effect will terminate. Such permanent changes usually will result in down-regulation. Another consideration is that in some cases one does not need a terribly long-term effect such as the application of one or more reversible non-invasive modalities for treatment of an acute condition such as acute pain related to a dental procedure or outpatient surgery.

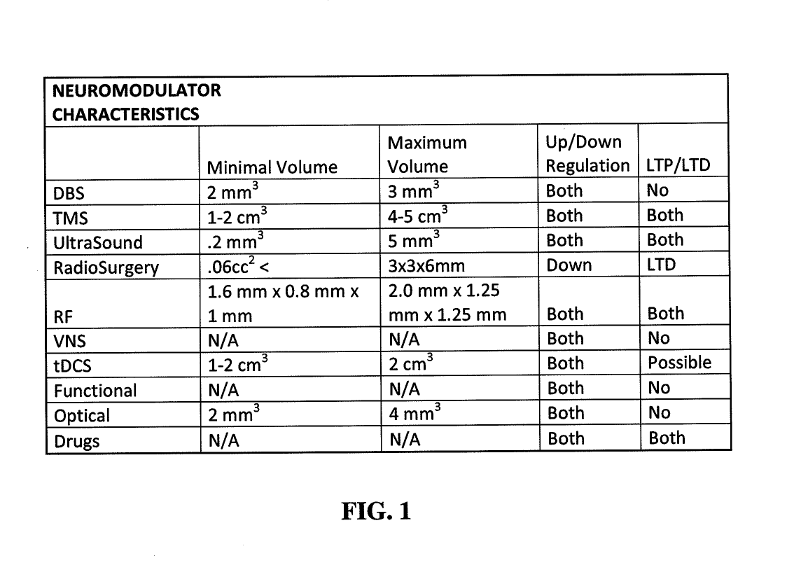

FIG. 1 shows the characteristics of the various neuromodulation modalities. The values for the parameters are approximate and not meant to be absolute. Which treatment modality is to be used in what position for what target depends on such factors as the size of the target (e.g., ultrasound can be focused to 0.5 to 2 mm³ while TMS can be limited to 1-2 cm³ at best), target accessibility, the presence of critical neural structures for which stimulation is to be avoided in proximity to the target, whether side effects will be elicited, local characteristics of the neural tissue (e.g., tDCS can only be used on superficial targets, DBS is not applicable to structures like the Insula that have a high degree of vascularity), whether up or up regulation is to be performed, whether Long-Term Potentiation (LTP) or Long-Term Depression (LTD) is desired, and whether there is physically enough room for the physical combination of neuromodulation elements. Another critical element is whether an invasive modality (e.g., DBS, VNS, optical) is acceptable or not. It is to be noted that radiosurgery can only down-regulate. A fundamental consideration of this invention that a given target may best targeted by one or a set of modalities. For example, a long structure like the DACG may be amenable to deep-brain TMS stimulation while a relatively small target such as the Nucleus Accumbens may be best targeted by DBS. Another consideration is that as the overall clinical therapeutic approach develops, one or more additional modalities may be considered at the point where one or more modalities are already in place. The principles of this invention are important and the invention is not limited to the currently available modalities, because existing techniques will be improved, new techniques will be discovered, and additional targets for given indications will be identified.

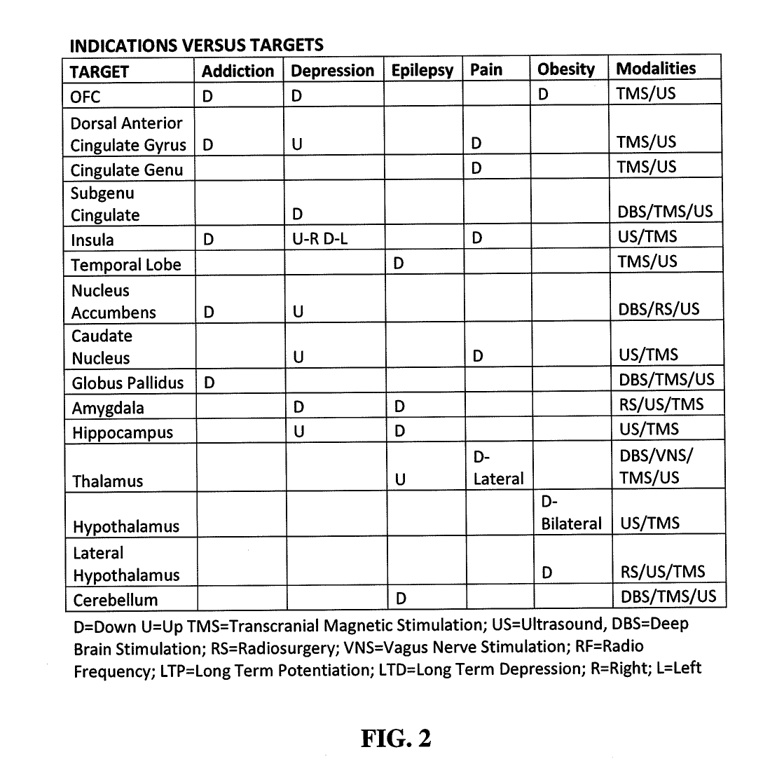

FIG. 2 is a table of Indications versus Targets. Many of these are shown on brainmaps.com. Not all targets for each indication is listed, only the main ones according to current understanding. As additional knowledge is discovered targets or which modality is or modalities are preferable may change. Not all the targets listed need to be hit for treatment to be effective. The entries in each of the indication columns represent either down-regulation (D) or up-regulation (U) for that given target for that indication. Not all targets will be regulated one way or the other for all indications. For example, the Dorsal Anterior Cingulate Gyrus (DACG) is up-regulated for depression and down-regulated for addiction and pain. Likely modalities are listed in the last column of the table. While there may be some preference for the order listed for a given modality according to one judgment the order is by no means mandatory. In some cases, the most effective combination may even be patient specific. In addition, it is possible that other modalities could be used effectively either instead of, or perhaps in addition to a listed modality. Depending on the target set, it may be that using a single modality may also work. An important consideration is that even though many targets are available, in practice one would not necessarily choose to hit all the targets but might well choose a subset. In some cases, there may be too many targets to permit all too be targeted so choices will need to be made. In other cases, it might be possible to set up a combined mechanism to hit all the targets, but it may be too expensive to do so relative to additional benefit to be obtained. In any case, new targets may be discovered as more knowledge is developed.

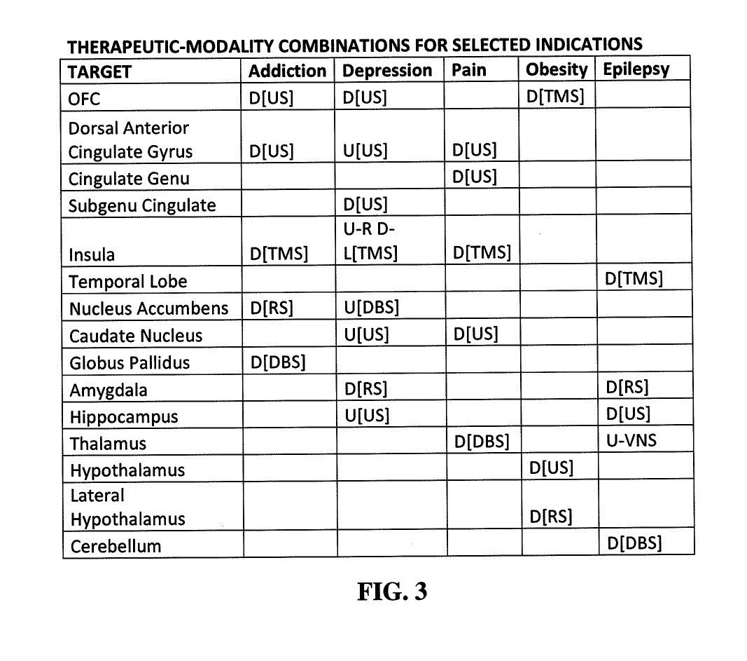

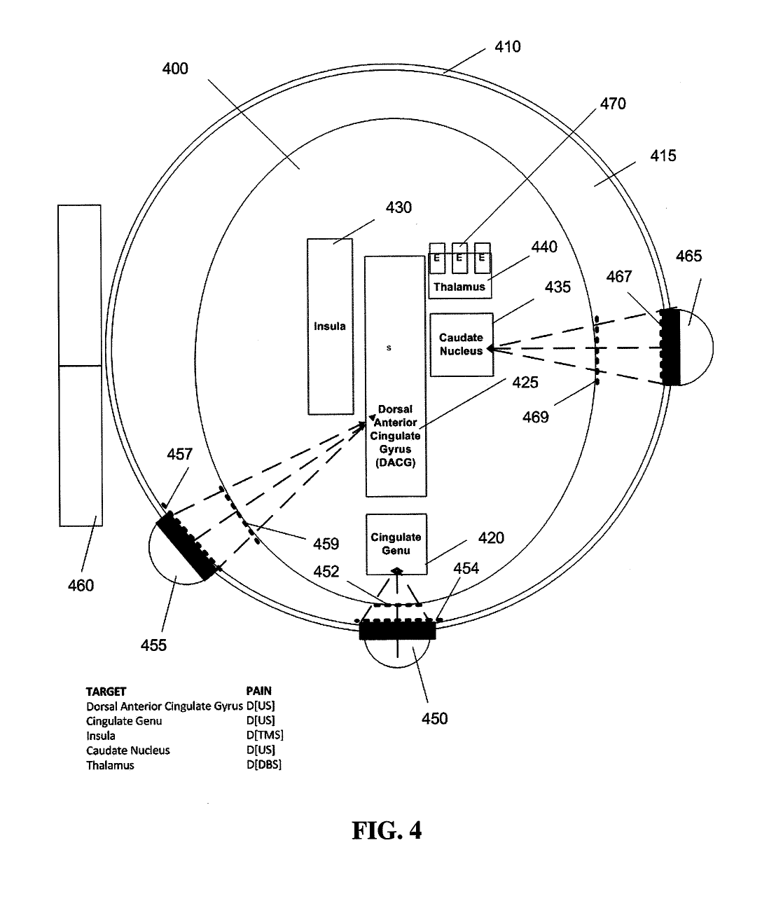

FIG. 3 shows a table for Therapeutic-Modality Combinations for Selected Indications. These represent one combination for each of the five covered indications, pain, depression, addiction, obesity, and epilepsy. The entries in each of the indication columns represent either down-regulation (D) or up-regulation (U) for that given target for that indication plus the particular therapeutic modality to be used. As shown in the diagrams for each seen in FIGS. 4 through 8, an important consideration is the physical space required for each of the energy sources. In some cases moving them off to a different plane and/or orientation may allow tighter packing.

FIG. 4 shows the physical layout of the combination of therapeutic modalities as listed in the table of FIG. 3 for the treatment of pain. The entries from that table just for pain are shown in the lower left-hand corner of the figure for reference. A frame 410 for holding energy sources surrounds head 400. The targets Cingulate Genu 420 neuromodulated by ultrasound transducer 450, Dorsal Anterior Cingulate Gyrus (DACG) 425 neuromodulated by ultrasound transducer 455, Insula 430 neuromodulated by TMS coil 460, Caudate Nucleus 435 neuromodulated by ultrasound source 465, and Thalamus 440 neuromodulated by DBS stimulating electrodes 470 are illustrated. In the case of ultrasonic transducers, the space between frame 410 and head 400 is filled with an ultrasonic conduction medium 415 such as Dermasol from California Medical Innovations with the interfaces between the head and the ultrasonic conduction medium and the ultrasonic medium and the ultrasound transducer are provided by layers of ultrasonic conduction gel, 452 and 454 for ultrasound transducer 450, 457 and 459 for ultrasound transducer 455, and 467 and 469 for ultrasound transducer 465. Note that while specific modalities for the targets are given, appropriate substitutions (i.e., target appropriate to modality, modality physically will fit with the mechanism for the other targets, etc.) can be made. Also, alternative targets to treat a given indication may be appropriate. The preceding points, while included on this section of pain, apply to the indications covered in the following paragraphs and other indications as well. For any of the indications the positions and orientations of the energy sources are set according to the particular needs of the targets and physical configuration. In another embodiment, more than one modality can be used to hit a single target to increase the effect. For example, both ultrasound and TMS could be used to simultaneously or sequentially hit the Dorsal Anterior Cingulate Gyms.

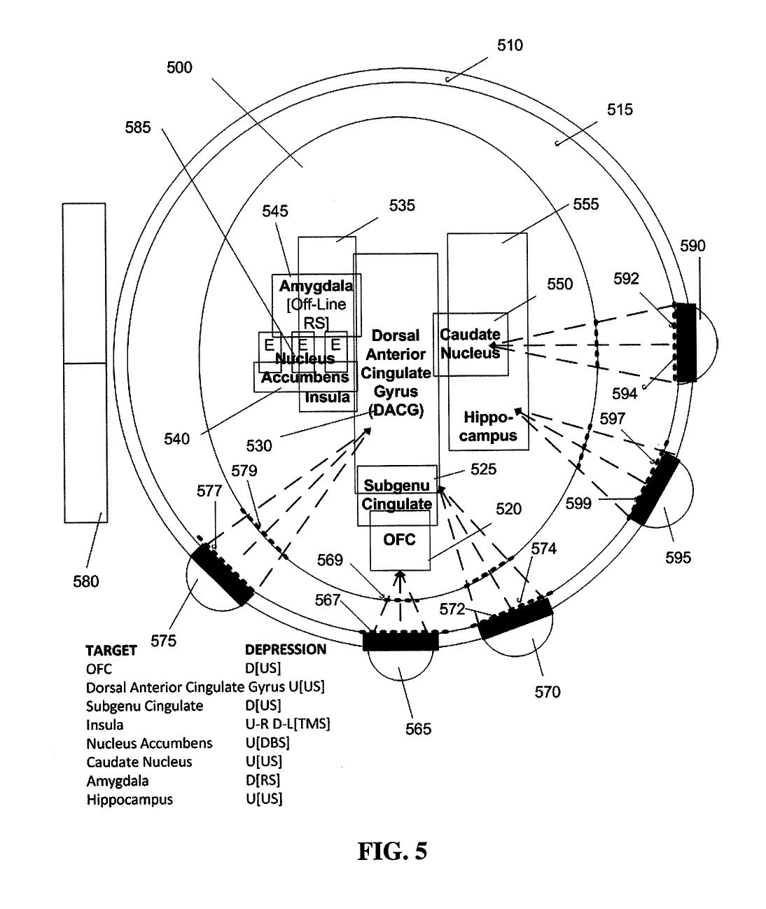

FIG. 5 shows the physical layout of the combination of therapeutic modalities as listed in the table of FIG. 3 for the treatment of depression. The entries from that table just for depression are shown in the lower left-hand corner of the figure for reference. A frame 510 for holding energy sources surrounds head 500. The targets OFC 520 neuromodulated by ultrasound transducer 565, Subgenu Cingulate 525 neuromodulated by ultrasound transducer 570, Dorsal Anterior Cingulate Gyrus (DACG) 530 neuromodulated by ultrasound transducer 575, Insula 535 neuromodulated by TMS coil 580, Nucleus Accumbens 540 neuromodulated by DBS stimulating electrodes 585, Amygdala 545 down-regulated by off-line radiosurgery, Caudate Nucleus 550 neuromodulated by ultrasound source 590, and Hippocampus 555 neuromodulated by ultrasound transducer 595 are illustrated. In the case of ultrasonic transducers, the space between frame 510 and head 500 is filled with an ultrasonic conduction medium 515 such as Dermasol from California Medical Innovations with the interfaces between the head and the ultrasonic conduction medium and the ultrasonic medium and the ultrasound transducer are provided by a layer of ultrasonic conduction gel, 567 and 569 for ultrasound transducer 565, 572 and 574 for ultrasound transducer 570, 577 and 579 for ultrasound transducer 575, and 592 and 594 for ultrasound transducer 590, and 597 and 599 for ultrasound transducer 595. A consideration is that embodiments with alternative configurations (e.g., one or multiple fewer targets) can work as well. It is to be noted that one would expect that additional targets will be discovered as more knowledge is gained so future additions or replacements are expected.

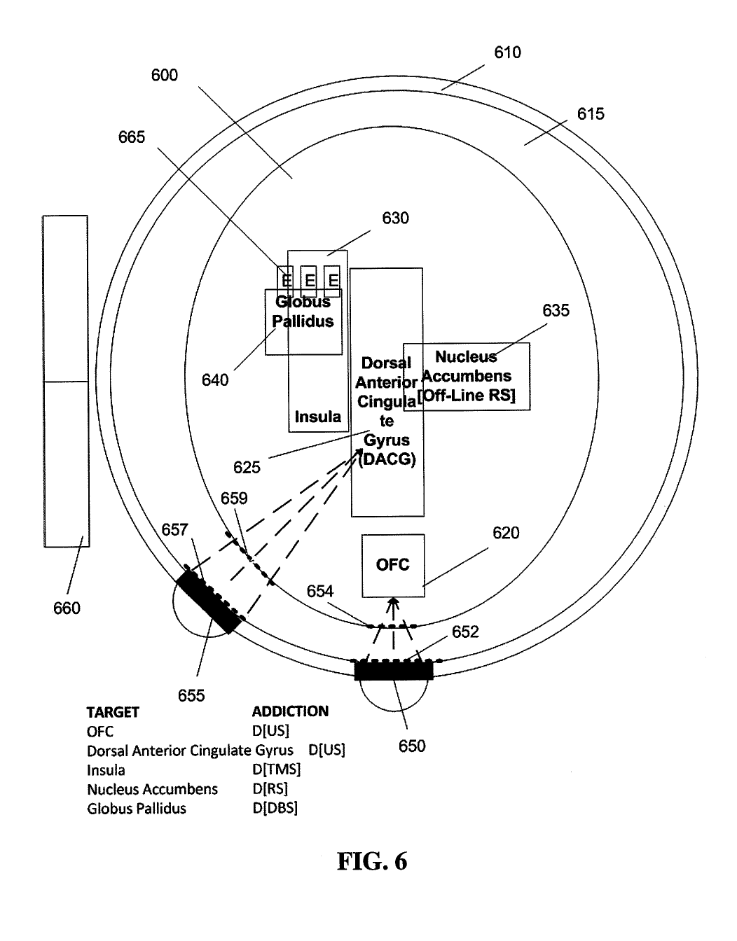

FIG. 6 shows the physical layout of the combination of therapeutic modalities as listed in the table of FIG. 3 for the treatment of addiction. The entries from that table just for addiction are shown in the lower left-hand corner of the figure for reference. A frame 610 for holding energy sources surrounds head 600. The targets OFC 620 neuromodulated by ultrasound transducer 650, Dorsal Anterior Cingulate Gyrus (DACG) 625 neuromodulated by ultrasound transducer 655, Insula 630 neuromodulated by TMS coil 660, Nucleus Accumbens 635 down-regulated by off-line radiosurgery, and Globus Pallidus 640 neuromodulated by DBS stimulating electrodes 665 are illustrated. In the case of ultrasonic transducers, the space between frame 610 and head 600 is filled with an ultrasonic conduction medium 615 such as Dermasol from California Medical Innovations with the interfaces between the head and the ultrasonic conduction medium and the ultrasonic medium and the ultrasound transducer are provided by a layer of ultrasonic conduction gel, 652 and 654 for ultrasound transducer 650, and 657 and 659 for ultrasound transducer 655. Note that in addiction that there are subgroups like smoking vs. drugs for which targets can vary.

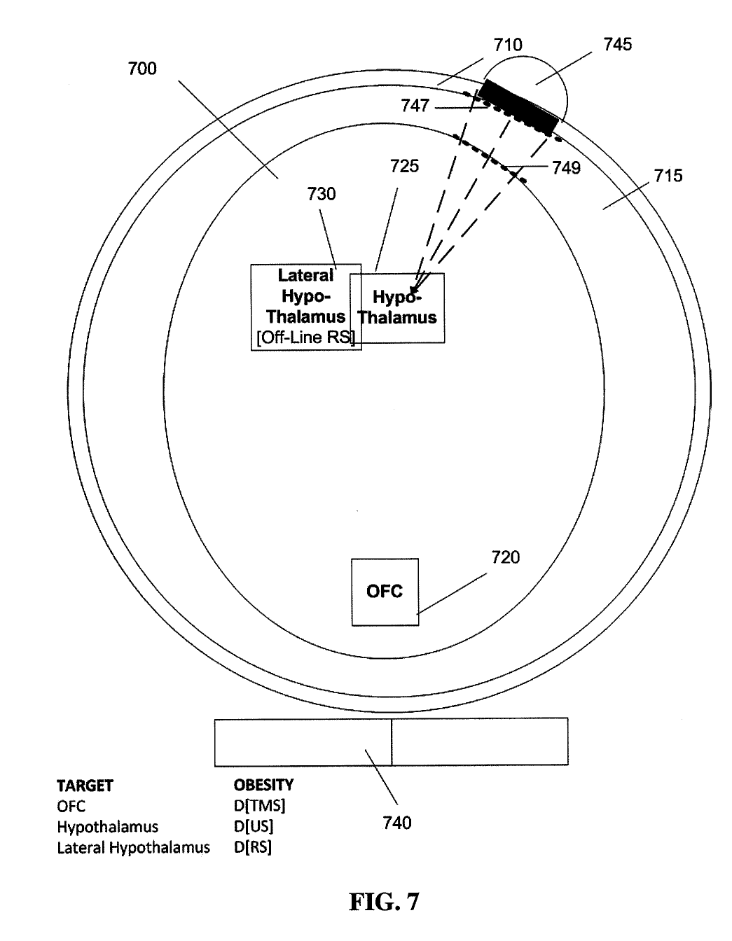

FIG. 7 shows the physical layout of the combination of therapeutic modalities as listed in the table of FIG. 3 for the treatment of obesity. The entries from that table just for obesity are shown in the lower left-hand corner of the figure for reference. A frame 710 for holding energy sources surrounds head 700. The targets OFC 720 neuromodulated by TMS coil 740, Hypothalamus 725 neuromodulated by ultrasound source 745, and Lateral Hypothalamus 730 down-regulated by off-line radiosurgery are illustrated. In the case of ultrasonic transducers, the space between frame 710 and head 700 is filled with an ultrasonic conduction medium 715 such as Dermasol from California Medical Innovations with the interfaces between the head and the ultrasonic conduction medium and the ultrasonic medium and the ultrasound transducer are provided by a layer of ultrasonic conduction gel, 747 and 749 for ultrasound transducer 745.

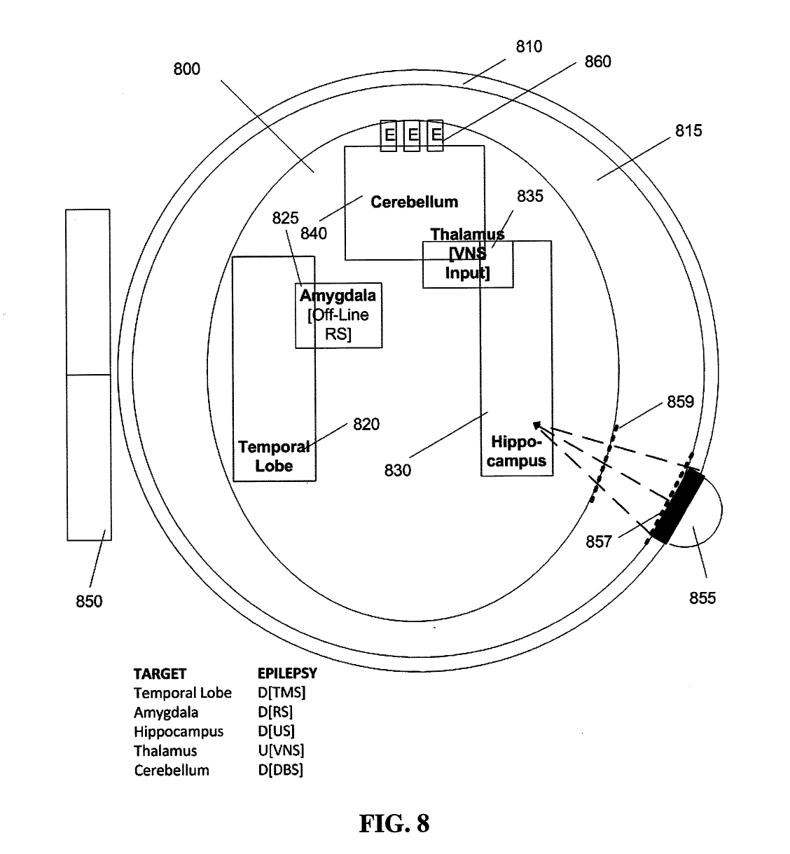

FIG. 8 shows the physical layout of the combination of therapeutic modalities as listed in the table of FIG. 3 for the treatment of epilepsy. The entries from that table just for epilepsy are shown in the lower left-hand corner of the figure for reference. A frame 810 for holding energy sources surrounds head 800. Targets Temporal Lobe 820 neuromodulated by TMS coil 850, Amygdala 825 down-regulated by off-line radiosurgery, Hippocampus 830 neuromodulated by ultrasound source 855, Thalamus 835 neuromodulated by VNS, and Cerebellum 840 neuromodulated by DBS stimulating electrodes 860 are illustrated. In the case of ultrasonic transducers, the space between frame 810 and head 800 is filled with an ultrasonic conduction medium 815 such as Dermasol from California Medical Innovations with the interfaces between the head and the ultrasonic conduction medium and the ultrasonic medium and the ultrasound transducer are provided by a layer of ultrasonic conduction gel, 857 and 859 for ultrasound transducer 855.

Note that where bilateral targets for any indication exist, both sides could be stimulated in other embodiments if the neuromodulation elements can be physically accommodated. Some embodiments may incorporate sequential rather than simultaneous application of on-line, real-time modalities such as ultrasound and TMS. In still other embodiments, multiple indications can be treated simultaneously or sequentially.

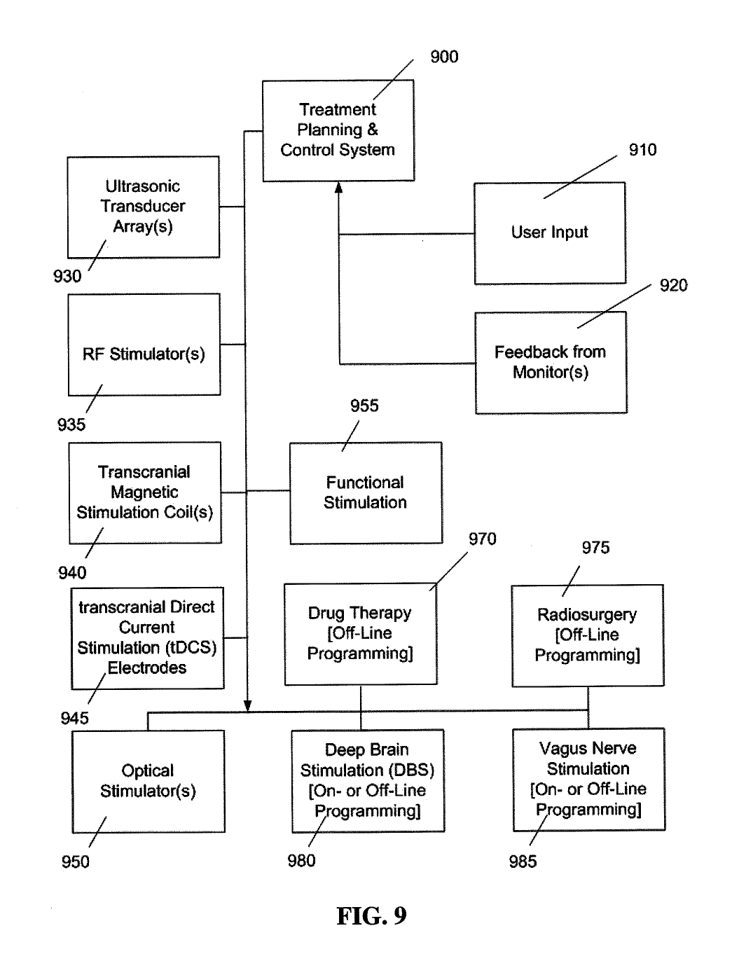

The targeting can be done with one or more of known external landmarks, an atlas-based approach (e.g., Tailarach or other atlas used in neurosurgery) or imaging. The imaging can be done as a one-time set-up or at each session although not using imaging or using it sparingly is a benefit, both functionally and the cost of administering the therapy, over approaches like Bystritsky (U.S. Pat. No. 7,283,861) which teaches consistent concurrent imaging. A block diagram is shown in FIG. 9 that depicts the Treatment Planning and Control System that has inputs from the user and monitoring systems (e.g., energy levels for one or more therapeutic modalities and imaging) and outputs to the various modalities. The treatment planning and control system varies, as applicable, the direction of energy emission, intensity, session duration, frequency, pulse-train duration, phase, firing patterns, numbers of sessions, and relationship to other controlled modalities. Use of ancillary monitoring or imaging to provide feedback is optional. Treatment Planning and Control System 900 receives input from User Input 910 and Feedback from Monitor(s) 920 and provides control output (either real-time or instructions for programming) to Transducer Array(s) 930, RF Stimulator(s) 935, Transcranial Magnetic Stimulation Coil(s) 940, transcranial Direct Current Stimulation (tDCS) Electrodes 945, Optical Simulator(s) 950, Functional Stimulation 955, Drug Therapy 970 [Off-Line Programming], Radiosurgery 975 [Off-Line Programming], Deep Brain Stimulation (DBS) 980 [On- or Off-Line Programming], and Vagus Nerve Stimulation (VNS) 985 [On- or Off-Line Programming] There are four categories of output modalities: [0037] a) on-line-real-time where neuromodulation parameters are changed immediately under direct control of the Treatment Planning and Control System (e.g., ultrasound transducers or TMS stimulators), [0038] b) on-line-prescriptive where neuromodulation parameters are directly set in programmers (e.g., DBS or Vagus Nerve Stimulation programmers) and the effect is both reversible and seen immediately, [0039] c) off-line-prescriptive-adjustable where instructions are generated for users to adjust drug dosages or adjust programmers and the effect is reversible but the effect is seen at a later time after the programmers (e.g., DBS or Vagus Nerve Stimulation programmers) have been so adjusted, and [0040] d) off-line-prescriptive-permanent where neuromodulation parameters are instructions are generated for users to adjust parameters and the effect is not reversible (e.g., radiosurgery) and the effect is seen at a later time after the change has been made. Examples of types of control exercised are positioning transducers, controlling pulse frequencies, session durations, numbers of sessions, pulse-train duration, firing patterns, and coordinating firing so that hitting of multiple targets in the neural circuit using firing patterns is done with optimal effects. In addition, in some cases, firing patterns (Mishelevich, D. J. and M. B. Schneider, “Firing Patterns for Deep Brain Transcranial Magnetic Stimulation,” PCT Patent Application PCT/US2008/073751, published as WIPO Patent Application WO/2009/026386) can be used where multiple energy sources of the same or different types are impacting a single target. This strategy can be used to avoid over-stimulating neural tissues between an energy source and the target to avoid undesirable side effects such as seizures. Positioning of neuromodulators and their settings may be patient specific in terms of (a) the actual position(s) of the target(s), (b) the neuromodulation parameters for the targets, and (c) the functional interactions among the targets. In some case performing imaging or other monitoring, may help in determining adjustments to be made, whether those adjustments are made manually or automatically.

In some cases, an off-line procedure will have already been permanently done (e.g., radiosurgery) and for that modality what occurred would only appear as an input. Control will involve such aspects such as the firing patterns that are employed in each of the applicable modalities, the pattern of stimulation among the employed modalities, and whether simultaneous or sequential neuromodulation is employed (including off-line modalities which will automatically mean sequential neuromodulation is done, if any of the therapeutic modalities in the combination are applied in real-time).

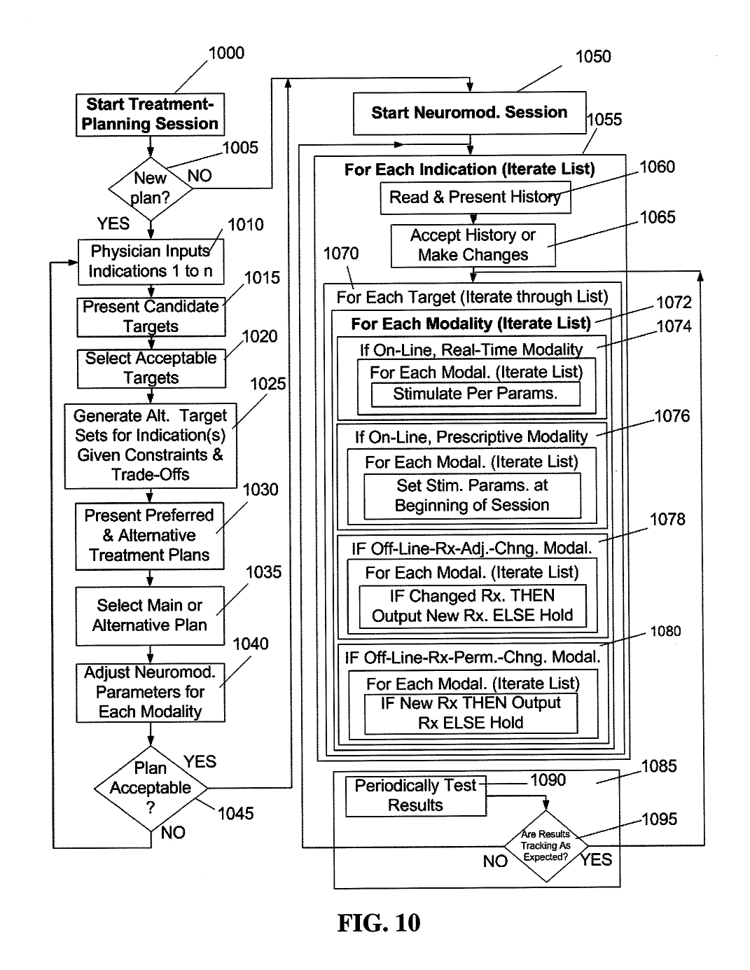

FIG. 10 illustrates the flow for the Treatment Planning and Control System. Just after the start of the Treatment-Planning Session 1000, a branch 1005 occurs which depending on whether this is a new plan (for a new patient) proceeds (if the result is yes) to the physician putting in the indications to be treated 1010 or proceeds (if the result is no) to the start of the Neuromodulation Session 1050.

The flow for the development of the new plan is for in 1010 the physician to input the desired indications followed by the presentation of candidate targets to the physician in 1015. There may be only a single indication. The physician selects the acceptable targets in 1020 and then the system generated alternative target sets associated with the selected indication(s) in 1025 given that physical constraints are satisfied. Trade-offs are given in terms of risk, anticipated relative benefits, possible side effects, and other factors. The resultant preferred treatment plan plus alternative plans are presented to the physician in 1030 and the physician makes the selection of what is to be done in 1035 and adjusts the neuromodulation parameters for each of the modalities in 1040. A branch 1045 follows related to whether the resultant plan is acceptable to the physician. If the answer is no, then the process is repeated with the physician again inputting the desired indications in 1010. If the answer is yes and the results plan is acceptable, then the Neuromodulation Session is started in 1050.

The Neuromodulation Session consists of iterating through each of the designated indications in 1055. For each indication, the system reads and presents the history in 1060 and the physician in 1065 accepts the historical values or makes changes. Then in 1070 the system iterates through each of the designated targets and, then within target, in 1072, the system iterates through each of the appropriate modalities. The actions depend on the category of the modality. If the case involves an On-Line, Real-Time Modality in 1074, the modalities are iterated through, and the given modality is stimulated according to the parameter set. If the case involves an On-Line Prescriptive Modality 1076, then for each of the modalities, the stimulation parameters are set in the given programmer at the beginning of the session. Not all programmers can be automatically set by another system such as the Multi-Modality Treatment-Planning and Control system of the invention, so this mechanism may not be available. In any case if such a modality (e.g., DNS or VNS) can be controlled in this way, the set stimulation will usually continue after the On-Line, Real-Time Modalities such as TMS or Ultrasound session is complete. If the case involves an Off-Line-Prescriptive-Adjustable-Change Modality 1078, then for each of the modalities the stimulation parameters for the programmer are changed if there is new prescription or held if there is not. Finally, if the case involves an Off-Line-Prescriptive-Change Modality, then for each of the modalities if there now is a prescription, the prescription is output; otherwise the prescription is held. There may be more than one such a modality of that type (e.g., two or more radiosurgery modalities), each related to a different target.

An evaluation of the results occurs in 1085. Periodically (either within a neuromodulation session or days, weeks, months, or perhaps even years apart) the functional results are tested in 1090. A branch 1095 is executed related to whether the results are tracking as expected. If the answer is no, then the flow returns to 1055 and each of the indications is iterated through including reading and presenting the history 1060 with physician accepting the historical parameter sets or altering them in 1065 prior to executing the overall program in 1070. If the answer is yes, then no parameter-set changes are required and the flow returns directly to executing the overall program in 1070.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity which can have benefits, for example, in the early treatment of head trauma or other insults to the brain.

A key aspect of the invention described above is that multiple conditions may be treated at the same time. This can be because the indications to be treated share a single target (e.g., the Dorsal Anterior Cingulate Gyrus (DACG) is down regulated in the treatment of both addiction and pain), or multiple targets in multiple circuit are neuromodulated. The treatment of multiple conditions is likely to become increasingly important as the average age of a given population increases. For example when stroke is being treated, in some cases, it will be practical to treat another condition as well. In treating indications with a common target, one most consider whether that target is neuromodulated in the same direction for both conditions. Otherwise, if for one condition the target is to be up-regulated and for the other condition the target is to be down-regulated, there is a conflict.

All of the embodiments above are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD). In addition, this approach can decrease the number of treatment sessions required for a demonstrated effect and to sustain a long-term effect. Follow-up tune-up sessions at one or more later times may be required.

The various embodiments described above are provided by way of illustration only and should not be construed to limit the invention(s) described above. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part II: Neuromodulation of Deep-Brain Targets Using Focused Ultrasound

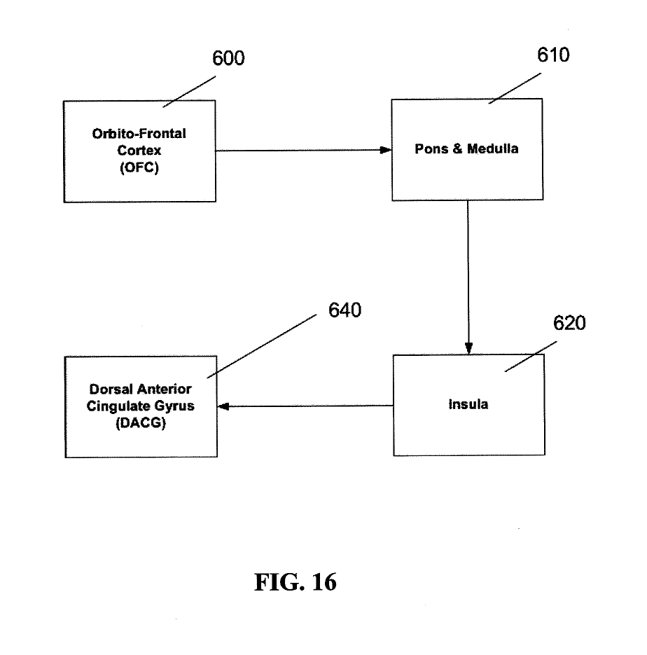

It is the purpose of some of the inventions described herein to provide methods and systems and methods for deep brain or superficial neuromodulation using ultrasound impacting one or multiple points in a neural circuit to produce acute effects or Long-Term Potentiation (LTP) or Long-Term Depression (LTD). For example, FIG. 16 illustrates the neural circuit for addiction.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down-regulated to achieve the desired effect, it may be preferable to up-regulate its reciprocal more-easily-accessed or safer reciprocal target instead. The frequency range allows penetration through the skull balanced with good neural-tissue absorption. In other embodiments, ultrasound therapy is combined with therapy using other neuromodulation devices (e.g., Transcranial Magnetic Stimulation (TMS), transcranial Direct Current Stimulation (tDCS), and/or Deep Brain Stimulation (DBS) using implanted electrodes). In other embodiments, ultrasound therapy is replaced with one or more therapies selected from one or more modalities of Radio-Frequency (RF) therapy, Transcranial Magnetic Stimulation (TMS), transcranial Direct Current Stimulation (tDCS), or Deep Brain Stimulation (DBS) using implanted electrodes.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 MHz, the value would be 2 mm Thus in the range of 0.4 MHz to 0.7 MHz, for this transducer, the spot sizes will be on the order of 5 mm at the low frequency and 2.8 mm at the high frequency.

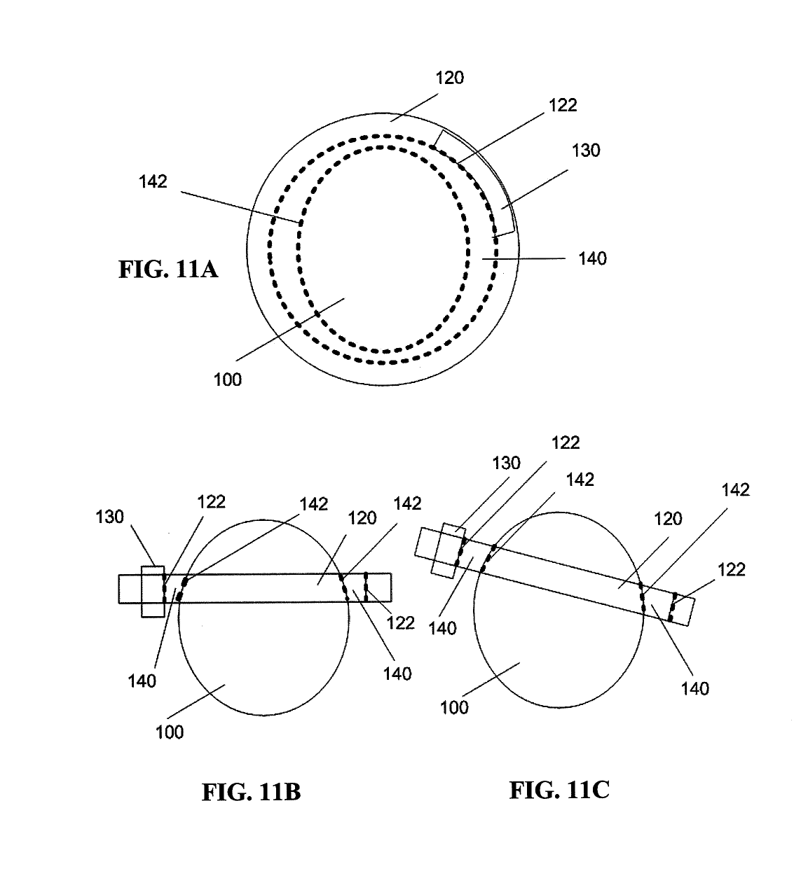

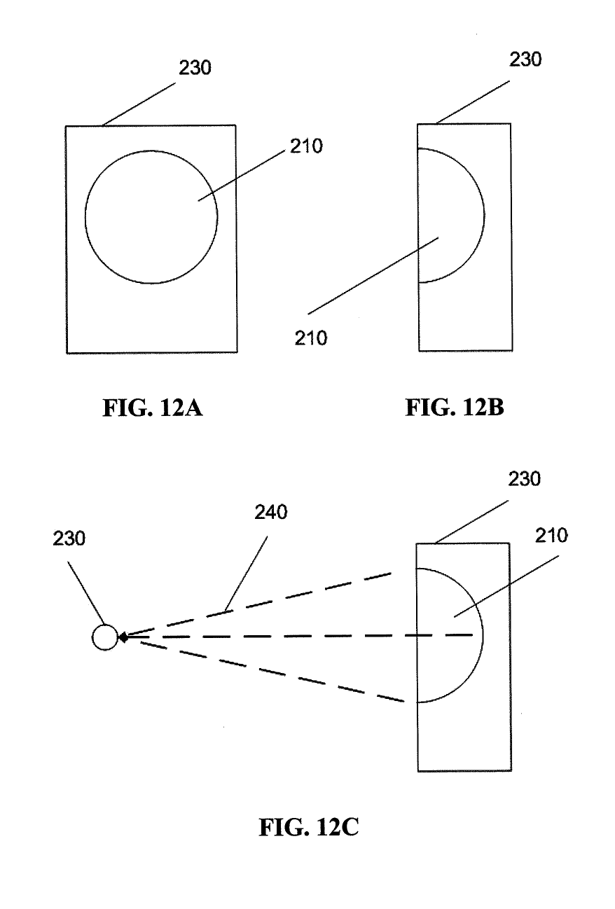

FIG. 11A shows the top view of one embodiment in which a track 120 surrounding human or animal head 100. Riding around track 120 is ultrasound transducer 130. In this embodiment, the face of transducer 130 always faces head 100. Track 120 includes rails for electrical connections to the ultrasound transducers 130. Transducer 130 can ride above the track 120, on the inside of the track 120, or below the track 120. In the latter case, the patient would have less of the apparatus covering their face. In some embodiments, more than one transducer 130 can ride on track 120. For the ultrasound to be effectively transmitted to and through the skull and to brain targets, coupling must be put into place. Ultrasound transmission medium (e.g., silicone oil in a containment pouch) 140 is interposed with one mechanical interface to the ultrasound transducer 130 (completed by a layer of ultrasound transmission gel 122) and the other mechanical interface to the head 100 (completed by a layer of ultrasound transmission gel 142). FIG. 11B shows the frontal view FIG. 11A for the case where transducer 130 is riding on the inside of track 120. The sound-conduction path between ultrasound transducer 130 and head 100 by conductive-gel layer 122, sound-conduction medium 140 and conductive-gel layer 142. FIG. 11C illustrates the situation where track 120 is tilted to allow better positioning for some targets or sets of targets if more than one neural structure is targeted in a given configuration. Again, ultrasound transmission medium 140 is interposed with one mechanical interface to the ultrasound transducer 130 (completed by a layer of ultrasound transmission gel 122) and the other mechanical interface to the head 100 (completed by a layer of ultrasound transmission gel 142). The depth of the point where the ultrasound is focused depends on the shape of the transducer and setting of the phase and amplitude relationships of the elements of the ultrasound transducer array discussed in relation to FIGS. 12A-12C. In another embodiment, a non-beam-steered-array ultrasound transducer can be used with the transducer only activated when it is correctly positioned to effectively aim at the target. As noted previously, in any case, the ultrasound transducer must be coupled to the head by an ultrasound transmission medium, including gel, if appropriate for effective ultrasound transmission can occur.

In another embodiment of the configuration shown in FIGS. 11A-11C, instead of the transducer or transducers 130 riding around on the track 120, they may fixed in place at a given location or locations on the track suitable to hit the desired target(s). In this case, in an alternative embodiment, a non-beam-steered-array ultrasound transducer can be used. Again, ultrasound transmission medium must be used for energy coupling.

FIGS. 12A-12C show the face of transducer 230 with an array of ultrasound transducers distributed over the face of transducer array assembly 210. FIG. 12A shows the front of the transducer as would face the target and FIG. 12B shows a side view. Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon in the U.S. is another custom-transducer supplier and Blatek is another. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations. FIG. 12C illustrates the ultrasound field represented by dashed lines 240 striking target neural structure 230 with the control of phase and amplitude producing the focus.

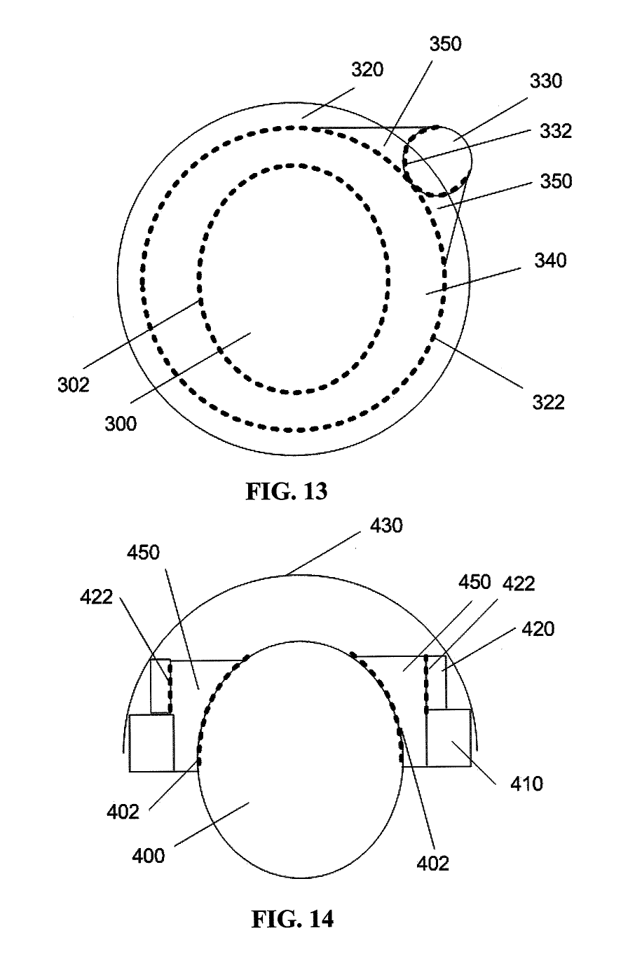

FIG. 13 illustrates an alternative embodiment where track 320 surrounds head 300 now has a transducer 330 whose face can be rotated so it can be aimed towards the intended target(s) rather than always facing perpendicularly to the head. Track 320 includes rails for electrical connections to the sound transducers 330. As transducer 330 reaches a given point on track 300, transducer 330 can be rotated toward the target(s). Again, in some embodiments, more than one transducer 330 can ride on track 320. For the ultrasound to be effectively transmitted to and through the skull and to brain targets, coupling must be put into place. Ultrasound transmission medium 340 is interposed with one mechanical interface to the ultrasound transducer 332 (completed by a layer of ultrasound transmission gel 322) and the other mechanical interface to the head 300 (completed by a layer of ultrasound transmission gel 302). For the rotating element 330, completion of the coupling is achieved with transmission coupling medium 350 is in place (completed by a layer of ultrasound transmission gel 322). In another embodiment, one or more transducers 330 can be fixed in position on track 320, but one or more of transducers 330 can still be rotated to it can be aimed towards the target. Such rotation can either allow sweeping over an elongated target or can periodically alternatively aimed toward each of more than one target. In some embodiments, one or more transducers fixed in position on the track are not rotated. The transducer arrays incorporated in transducer 130 in FIGS. 11A-11C and 330 in FIG. 13 can both of the form of FIGS. 12A-12C or other suitable configuration. In addition the tracks in the configurations shown in FIGS. 11A-C, FIG. 13 and their alternative embodiments can be raised and lowered vertically as required for optimal targeting. The track can be tilted side to side, front to back, diagonal, or in any direction according to the targeting need. The tracks can be tilted back and forth according to the targeting need. Also there may be transducer carriers containing a plurality of transducers so the combination can target more than one target simultaneously. Other embodiments may be smaller versions covering only a portion of the skull with the ability to target fewer (simultaneously) or perhaps only one target that can be used both in an increased number of clinical settings or at home. Another embodiment incorporates a transducer-holding device, which is not a track, which holds the ultrasound transducers in fixed positions relative to the target or targets. The locations and orientations of the holders can be calculated by locating the applicable targets relative to atlases of brain structure such as the Tailarach atlas. As noted above, in each case, transmission coupling medium must be in place.

In another embodiment, either of the implementations in FIGS. 11A-11C or FIG. 13 can be enclosed in a shell as shown in FIG. 14 where head 400 is shown in a frontal view with transducer 420 riding on track 410 all enclosed in shell 430. In this embodiment, there are two transducers 420, placed 180 degrees apart. In this case, as for the other configurations, for the effective ultrasound transmission to and through the skull and to brain targets, coupling must be put into place. Ultrasound transmission medium 450 is interposed with one mechanical interface to the ultrasound transducer 420 (completed by a layer of ultrasound transmission gel 422) and the other mechanical interface to the head 400 (completed by a layer of ultrasound transmission gel 402). In another embodiment, mechanical perturbations are applied radially or axially to move the ultrasound transducers. This is applicable to a variety of transducer configurations.

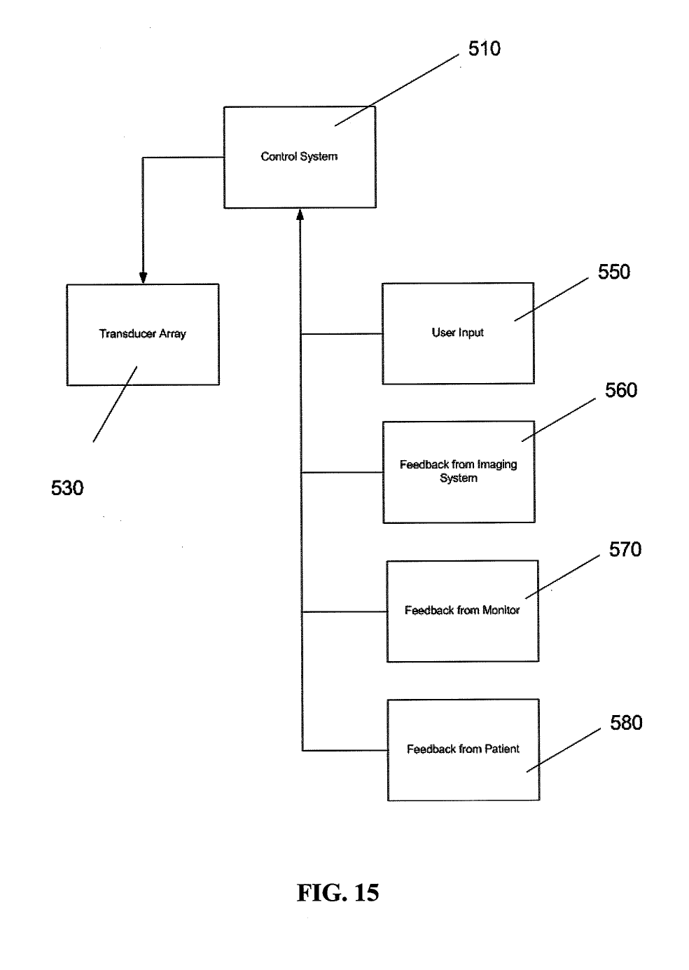

FIG. 15 shows an embodiment of a control circuit. The positioning and emission characteristics of transducer array 530 are controlled by control system 510 with control input from either user by user input 550 and/or from feedback from imaging system 560 (either automatically or display to the user with actual control through user input 550) and/or feedback from a monitor (sound and/or thermal) 570, and/or the patient 580. Control can be provided, as applicable, for direction of the energy emission, intensity, frequency for up-regulation or down-regulation, firing patterns, and phase/intensity relationships for beam steering and focusing on neural targets.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity which can have benefits, for example, in the early treatment of head trauma or other insults to the brain. An example of a neural circuit for a condition, in this case addiction is shown in FIG. 16. In this circuit, the elements are Orbito-Frontal Cortex (OFC) 600, Pons & Medulla 610, Insula 620, and Dorsal Anterior Cingulate Gyms (DACG) 640. One or more targets can be targeted simultaneously or sequentially. Down regulation means that the firing rate of the neural target has its firing rate decreased and thus is inhibited and up regulation means that the firing rate of the neural target has its firing rate increased and thus is excited. For the treatment of addiction, the OFC 600, Insula 620, and DACG 640 would all be down regulated. The ultrasonic firing/timing patterns can be tailored to the response type of a target or the various targets hit within a given neural circuit.

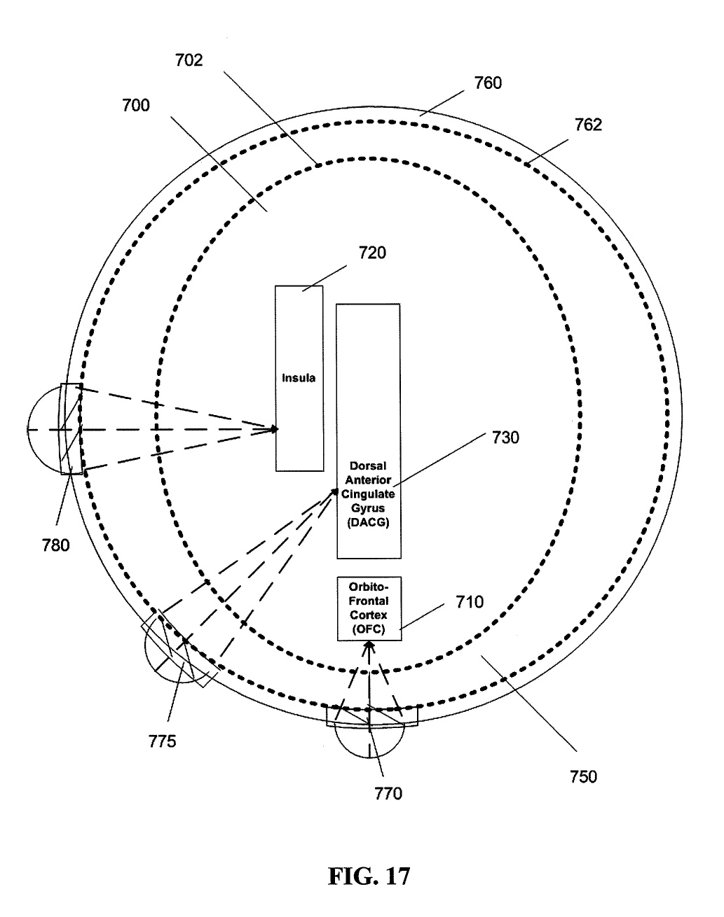

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) and enhances acute effects as well. In addition, this approach can decrease the number of treatment sessions required for a demonstrated effect and to sustain a long-term effect. Follow-up tune-up sessions at one or more later times may be required. FIG. 17 shows a multi-target configuration. The head 700 contains the three targets, Orbito-Frontal Cortex (OFC) 710, Insula 720, and Dorsal Anterior Cingulate Gyms (DACG) 730, also shown in FIG. 16. These targets are hit by ultrasound transducers 770, 775, and 780, running around track 760 or fixed to track 760. Ultrasound transducer 770 is shown targeting the OFC, transducer 775 is shown targeting the DACG, and transducer 780 is shown targeting the Insula. For the ultrasound to be effectively transmitted to and through the skull and to brain targets, coupling must be put into place. Ultrasound transmission medium 750 is interposed with one mechanical interface to the ultrasound transducers 770, 775, 780 (completed by a layer of ultrasound transmission gel 762) and the other mechanical interface to the head 700 (completed by a layer of ultrasound transmission gel 702). In some cases, the neural structures will be targeted bilaterally (e.g., both the right and the left Insula) and in some cases only one will targeted (e.g., the right Insula in the case of addiction).

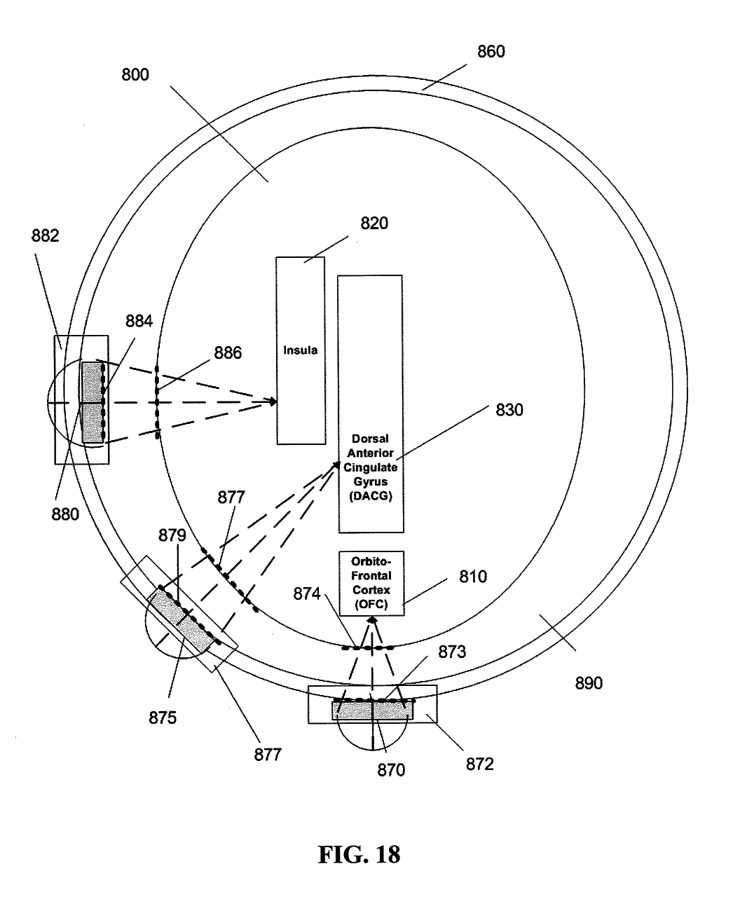

FIG. 18 shows a fixed configuration where the appropriate radial (in-out) positions have determined through patient-specific imaging (e.g., PET or fMRI) and the holders positioning the ultrasound transducers are fixed in the determined positions. The head 800 contains the three targets, Orbito-Frontal Cortex (OFC) 810, Insula 820, and Dorsal Anterior Cingulate Gyms (DACG) 830. These targets are hit by ultrasound transducers 870, 875, and 880, fixed to track 860. Ultrasound transducer 870 is shown targeting the OFC, transducer 875 is shown targeting the DACG, and transducer 880 is shown targeting the Insula. Transducer 870 is moved radially in or out of holder 872 and fixed into position. In like manner, transducer 875 is moved radially in or out of holder 877 and fixed into position and transducer 880 is moved radially in or out of holder 882 and fixed into position. For ultrasound to be effectively transmitted to and through the skull and to brain targets, coupling must be put into place. Ultrasound transmission medium 890 is interposed with one mechanical interface to the ultrasound transducers 870, 875, 880 (completed by a layers of ultrasound transmission gel 873, 879, 884) and the other mechanical interface to the head 800 (completed by a layers of ultrasound transmission gel 874, 877, 886). To support this embodiment, treatment planning software is used taking the image-determined target positions and output instructions for manual or computer-aided manufacture of the holders. Alternatively positioning instructions can be output for the operator to position the blocks holding the transducers to be correctly placed relative to the support track. In one embodiment, the transducers positioned using this methodology can be aimed up or down and/or left or right for correct flexible targeting.

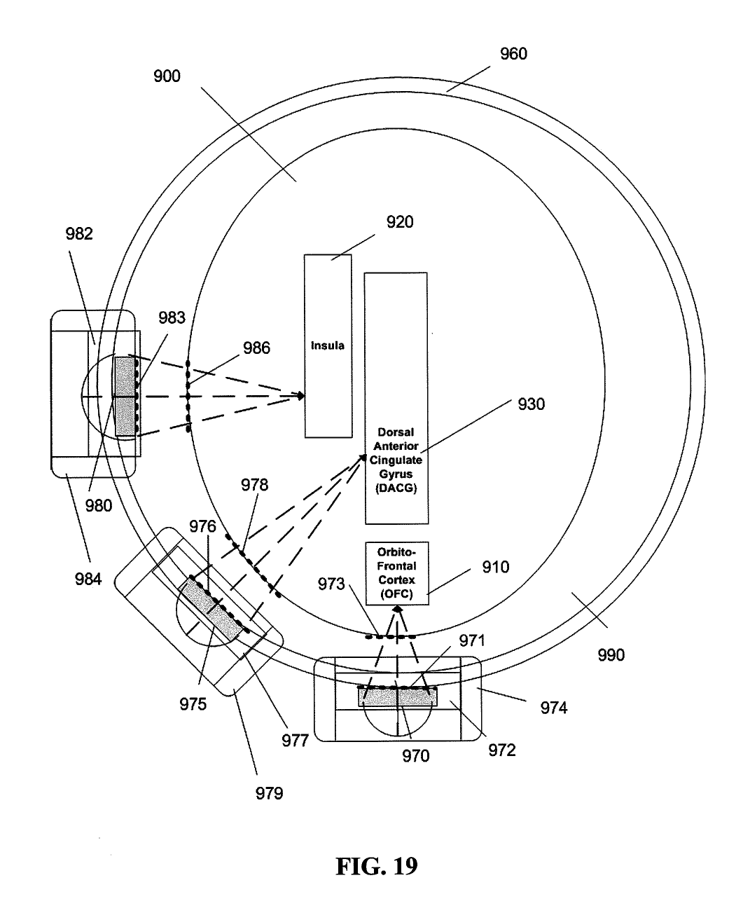

FIG. 19 illustrates an automatically adjustable configuration where based on the image-determined target positions discussed relative to FIG. 18, the transducer holders are moved in or out to the correct positions for the given target without a fixed patient-specific holder having been fabricated or manually adjusted relative to the track or other frame. The head 900 contains the three targets, Orbito-Frontal Cortex (OFC) 910, Insula 920, and Dorsal Anterior Cingulate Gyms (DACG) 930, also shown in FIG. 16. These targets are hit by ultrasound transducers 970, 975, and 980, fixed to track 960. Transducer 970 mounted on support 972 is moved radially in or out of holder 974 by a motor (not shown) to the correct position under control of treatment planning software or manual control. In like manner, transducer 975 mounted on support 977 is moved radially in or out of holder 979 by a motor (not shown) to the correct position under control of treatment planning software or manual control. In like manner, transducer 980 mounted on support 982 is moved radially in or out of holder 984 by a motor (not shown) to the correct position under control of the treatment planning software or manual control. Ultrasound transducer 970 is shown targeting the OFC, transducer 975 is shown targeting the DACG, and transducer 980 is shown targeting the Insula. For the ultrasound to be effectively transmitted to and through the skull and to brain targets, coupling must be put into place. Ultrasound transmission medium 990 is interposed with one mechanical interface to the ultrasound transducers 970, 975, 980 (completed by a layers of ultrasound transmission gels 971, 976, 983) and the other mechanical interface to the head 900 (completed by a layers of ultrasound transmission gel 973, 978, and 986). An embodiment involving the latter would use a single or fewer-than-the-number-of-targets transducers to hit multiple targets since the or fewer-than-the-number-of-targets transducers can be moved in and out or rotated left and right and/or up and down to hit the multiple targets.

The invention allows stimulation adjustments in variables such as, but not limited to, intensity, firing pattern, frequency, phase/intensity relationships, dynamic sweeps, and position to be adjusted so that if a target is in two neuronal circuits the transducer or transducers can be adjusted to get the desired effect and avoid side effects. The side effects could occur because for one indication the given target should be up-regulated and for the other down-regulated. An example is where a target or a nearby target would be down-regulated for one indication such as pain, but up-regulated for another indication such as depression. This scenario applies to either the Dorsal Anterior Cingulate Gyms (DACG) or Caudate Nucleus. Even when a common target is neuromodulated, adjustment of stimulation parameters may moderate or eliminate a problem because of differential effects on the target relative to the involved clinical indications.

The invention also contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down-regulated for one indication such as pain, but up-regulated for another indication such as depression. This scenario applies to the Dorsal Anterior Cingulate Gyms (DACG). To counterbalance the down-regulation of the DACG during treatment for pain that negatively impacts the treatment for depression, one would up-regulate the Nucleus Accumbens or Hippocampus which are other targets in the depression neural circuit. A plurality of such applicable targets could be stimulated as well.

Another applicable scenario is the Nucleus Accumbens which is down-regulated to treat addiction, but up-regulated to treat depression. To counteract the down-regulation of the Nucleus Accumbens to treat depression but will negatively impact the treatment of depression which would like the Nucleus Accumbens to be up-regulated, one would up-regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part III: Patient Feedback for Control of Ultrasound Deep-Brain Neuromodulation

It is the purpose of some of the inventions described herein to provide methods and systems for the adjustment of deep brain or superficial neuromodulation using ultrasound or other non-invasive modalities to impact one or multiple points in a neural circuit under patient-feedback control.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), Deep Brain Stimulation (DBS) using implanted electrodes, implanted optical stimulation, stereotactic radiosurgery, Radio-Frequency (RF) stimulation, vagus nerve stimulation, other local stimulation, or functional stimulation).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency. Spot size being smallest is not necessarily the most advantageous; what is optimal depends on the shape of the target neural structure. Such vendors as Keramos-Etalon and Blatek in the U.S., and Imasonic in France can supply suitable ultrasound transducers.

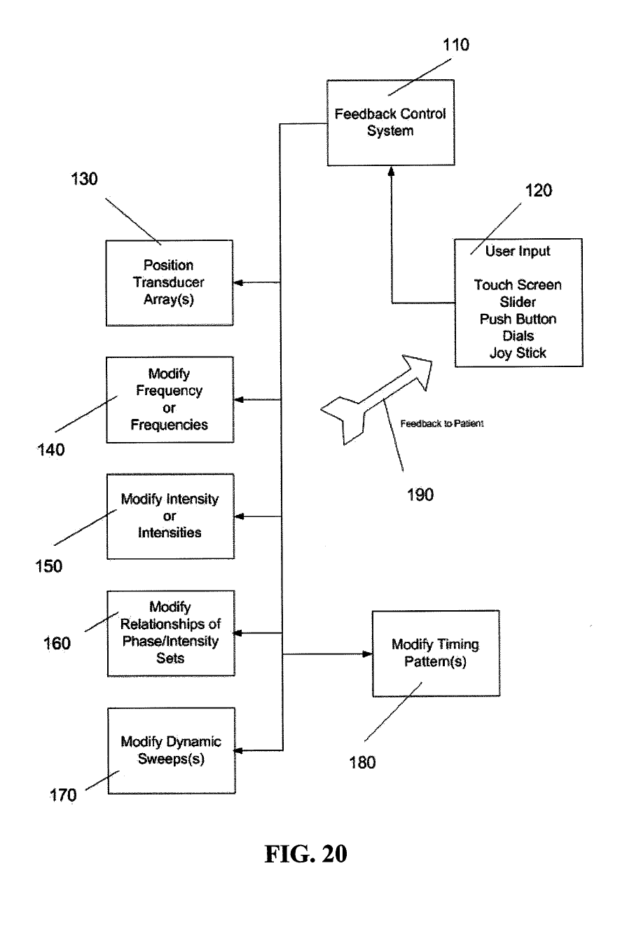

FIG. 20 shows the basic feedback circuit. Feedback Control System 110 receives its input from User Input 120 and provides control output for positioning ultrasound transducer arrays 130, modifying pulse frequency or frequencies 140, modifying intensity or intensities 150, modifying relationships of phase/intensity sets 160 for focusing including spot positioning via beam steering, modifying dynamic sweep patterns 170, and or modifying timing patterns 180. Feedback to the patient 190 occurs with what is the physiological effect on the patient (for example increase or decrease in pain or decrease or increase on tremor. User Input 120 can be provided via a touch screen, slider, dials, joystick, or other suitable means.

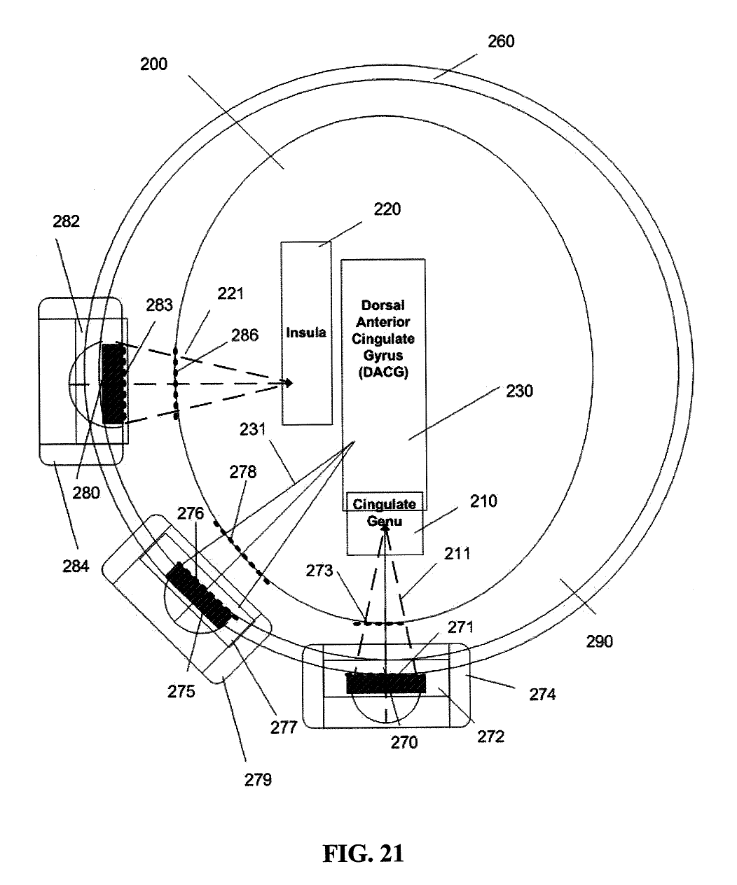

An example of a multi-target neural circuit related to the processing of pain sensation is shown in FIG. 21. Surrounding patient head 200 is ultrasound conduction medium 290, and ultrasound-transducer holding frame 260. Attached to frame 260 are transducer holders 274, 279, 284. These are oriented towards neural targets respectively holder 274 towards the Cingulate Genu 210, holder 279 towards the Dorsal Anterior Cingulate Gyms (DACG) 230, and holder 284 towards Insula 220. The assembly targeting Cingulate Genu 210, includes transducer holder 274 containing transducer 270 mounted on support 272 (possibly moved in and out via a motor (not shown)) with ultrasound field 211 transmitted though ultrasound conducting gel layer 271, ultrasound conducting medium 290 and conducting gel layer 273 against the exterior of the head 200. Examples of sound-conduction media are Dermasol from California Medical Innovations or silicone oil in a containment pouch.

The assembly targeting Dorsal Anterior Cingulate Gyms 230, includes transducer holder 279 containing transducer 275 mounted on support 277 (possibly moved in and out via a motor (not shown)) with ultrasound field 231 transmitted though ultrasound conducting gel layer 276, ultrasound conducting medium 290 and conducting gel layer 278 against the exterior of the head 200.

The assembly targeting Insula 220, includes transducer holder 284 containing transducer 280 mounted on support 282 (possibly moved in and out via a motor (not shown)) with ultrasound field 221 transmitted though ultrasound conducting gel layer 283, ultrasound conducting medium 290 and conducting gel layer 286 against the exterior of the head 200.

The locations and orientations of the holders 274, 279, 284 can be calculated by locating the applicable targets relative to atlases of brain structure such as the Tailarach atlas or via imaging (e.g., fMRI or PET) of the specific patient.

The invention can be applied to a number of conditions including, but not limited to, pain, Parkinson's Disease, depression, bipolar disorder, tinnitus, addiction, OCD, Tourette's Syndrome, ticks, cognitive enhancement, hedonic stimulation, diagnostic applications, and research functions.

One or more targets can be targeted simultaneously or sequentially. Down regulation means that the firing rate of the neural target has its firing rate decreased and thus is inhibited and up regulation means that the firing rate of the neural target has its firing rate increased and thus is excited. With reference to FIG. 21 for the treatment of pain, the Cingulate Genu 210, and DACG 230, and Insula 220 would all be down regulated. The ultrasonic firing patterns can be tailored to the response type of a target or the various targets hit within a given neural circuit.

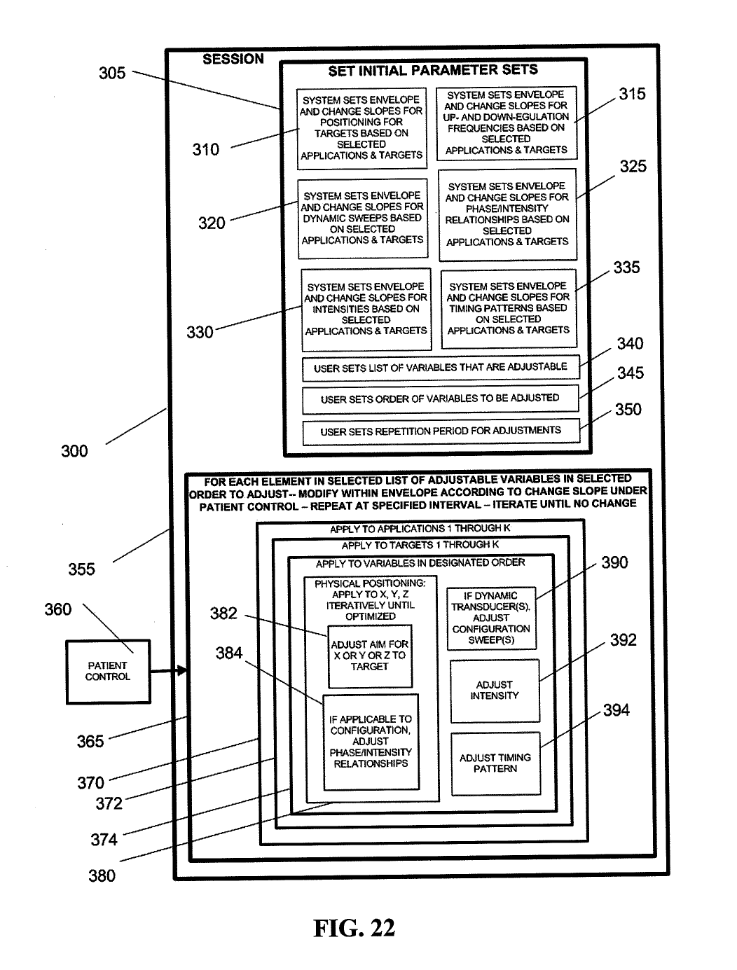

FIG. 22 shows an algorithm for processing feedback from the patient to control the ultrasound neuromodulation during a session 300. Before the real-time session begins, the initial parameters sets are set 305 by the system. This can be automatically, by the user healthcare professional instructing the system, or a combination of the two. These include setting the envelope and change slopes based on selected applications and targets for positioning for targets 310, up- and down-regulation frequencies 315, sweeps for dynamic transducers 320, phase/intensity relationships 325, intensities 330, and timing patterns 335. These are followed by the user setting what is to be controlled by the patient during the real-time feedback, namely list of variables that are adjustable 340, order of those variables to be adjusted 345, and repetition period for adjustments 350.

Once the initialization is complete the real-time part of the session begins based on patient-controlled input 360 (e.g., via touch screen, slider, dials, joy stick, or other suitable mean). During real-time processing, the outer loop 365 applies for each element in selected list of adjustable variables in selected order to adjust a modification within the envelope according to the change slope under patient control with repetition at the specified interval with iteration until there is no change felt by the patient. The process includes applying to applications 1 through k 370, applying to targets 1 through k 372, applying to variables in designated order 374, physical positioning (iteratively for x, y, z) 380 including adjusting aim towards target 382 and, if applicable to configuration, adjust phase/intensity relationships 384, in addition to adjustment of configuration sweeps if there is/are dynamic transducer(s) 390, adjust intensity 392, and adjusting timing pattern 394.

In like manner, patient-feedback control of other modalities is possible such as control of deep-brain stimulators (DBS) using implanted electrodes, Transcranial Magnetic Stimulation (TMS), transcranial Direct Current Stimulation (tDCS), implanted optical stimulation, radio-Frequency (RF) stimulation, Sphenopalatine Ganglion Stimulation, other local stimulation, or Vagus Nerve Stimulation (VNS).

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part IV: Shaped and Steered Ultrasound for Deep-Brain Neuromodulation

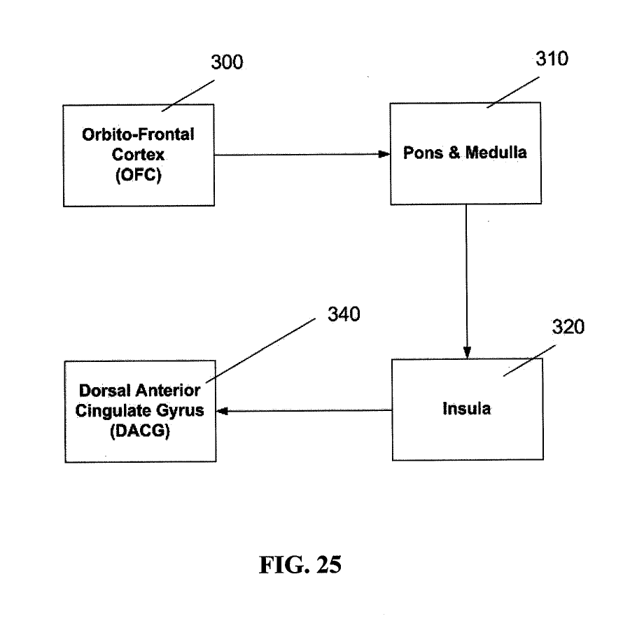

It is the purpose of some of the inventions described herein to provide a device for producing shaped or steered ultrasound for non-invasive deep brain or superficial stimulation impacting one or multiple points in a neural circuit to produce acute effects or Long-Term Potentiation (LTP) or Long-Term Depression (LTD) using up-regulation or down-regulation. For example, FIG. 25 illustrates the neural circuit for addiction.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of sound transducers of 300 or more. Keramos-Etalon and Blatek in the U.S. are other custom-transducer suppliers. The power applied will determine whether the ultrasound is high intensity or low intensity (or medium intensity) and because the sound transducers are custom, any mechanical or electrical changes can be made, if and as required.

The locations and orientations of the transducers in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. Using a phased array with ability to focus and steer the beam, two or more targets can be targeted sequentially. The ultrasonic firing patterns can be tailored to the response type of a target or the various targets hit within a given neural circuit.

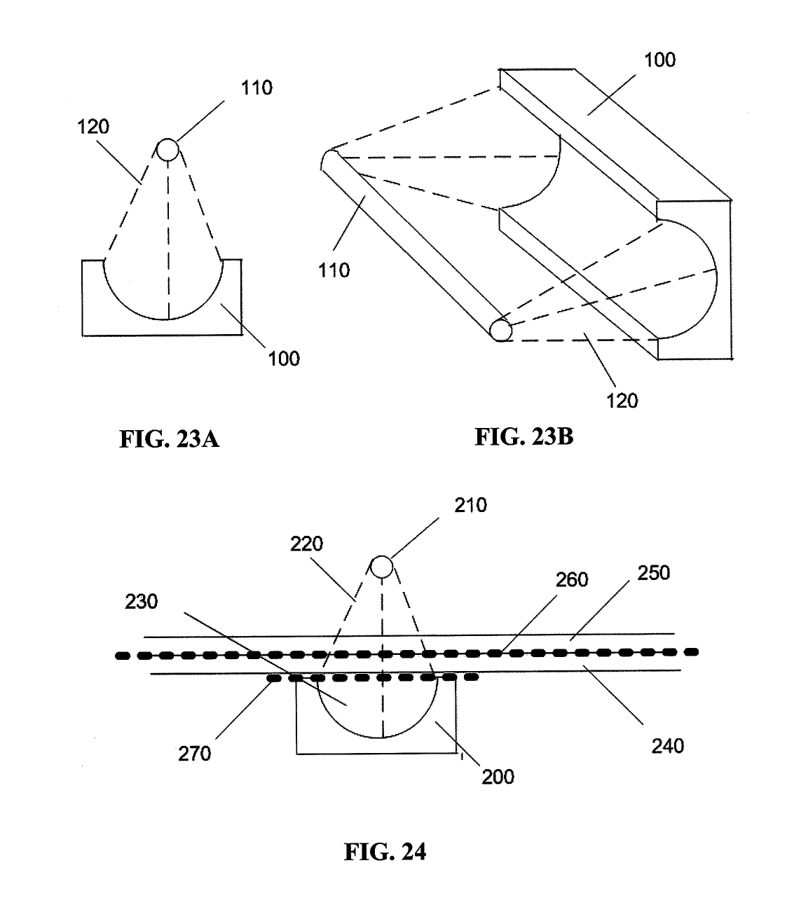

FIGS. 23A-23B show an ultrasound transducer array configured to produce an elongated pencil-shaped focused field. Such an array would he applied to stimulate an elongated target such as the Dorsal Anterior Cingulate Gyms (DACG) or the Insula. Note that one embodiment is a swept-beam transducer with the capability of sweeping the sound field over any portion of the length of the ultrasound transducer. Thus it is possible to determine over what length of a target that the ultrasound is applied. For example, one could apply ultrasound to only the anterior portion of the target. Also, by rotating or tilting a transducer in a holder, one can vertically target such as aiming the sound field at the superior portion of a target. In FIG. 23A, an end view of the array is shown with curved-cross section ultrasonic array 100 forming a sound field 120 focused on target 110. FIG. 23B shows the same array in a side view, again with ultrasound array 100, target 110, and focused field 120.

FIG. 24 illustrates the elongated ultrasound transducer array shown in FIGS. 23A-23B (now with ultrasound-transducer array 200, target 210, and focused ultrasound field 220), but in this case showing head layer 250 and sound-conduction medium 230 in place. Ultrasound is transmitted through fitted sound-conduction medium 230, a layer of conduction gel 270 providing the interface to solid sound-conduction medium 240, and a layer of conduction gel 260 providing interface to the head layer. Examples of sound-conduction media are Dermasol from California Medical Innovations or silicone oil in a containment pouch.

An example of a neural circuit for addiction is shown in FIG. 25. In this circuit, the elements are Orbito-Frontal Cortex (OFC) 300, Pons & Medulla 310, Insula 320, and Dorsal Anterior Cingulate Gyms (DACG) 340. One or more targets can be targeted simultaneously or sequentially. Down regulation means that the firing rate of the neural target has its firing rate decreased and thus is inhibited and up regulation means that the firing rate of the neural target has its firing rate increased and thus is excited. For the treatment of addiction, the OFC 300, Insula 320, and DACG 340 would all be down regulated. The ultrasonic firing patterns can be tailored to the response type of a target or the various targets hit within a given neural circuit.

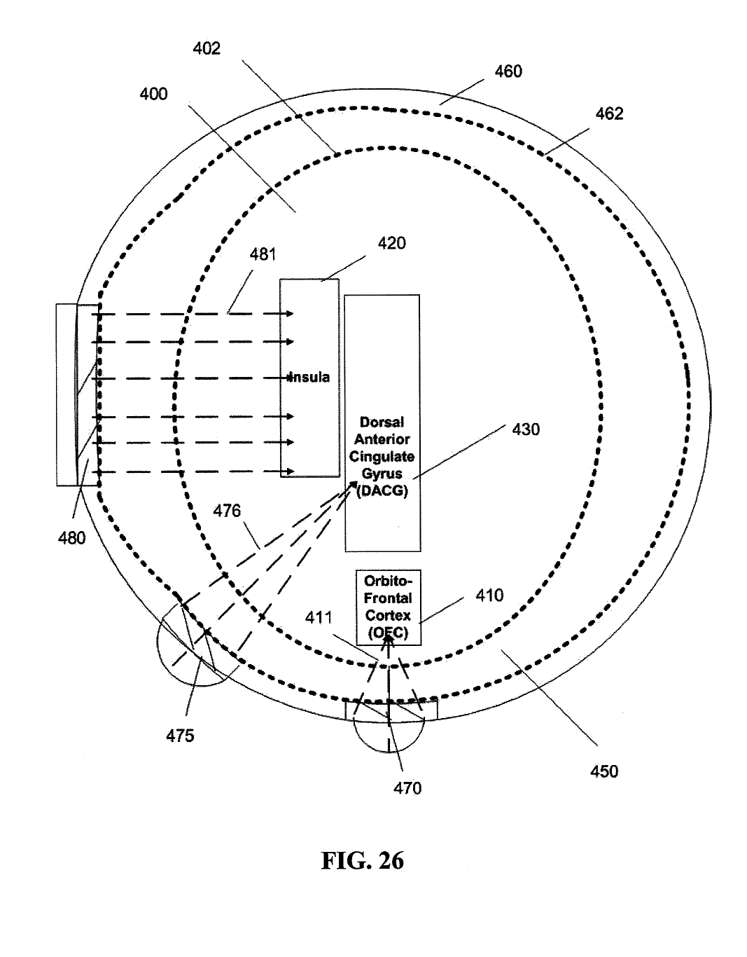

In FIG. 26, the physical target layout for addiction for the targets shown in FIG. 25 has within head 400 targets Orbito-Frontal Cortex (OFC) 410, Dorsal Anterior Cingulate Gyms (DACG) 430, and Insula 420. Sound field 411 emanating from ultrasound transducer 470 is focused on Orbito-Frontal Cortex (OFC) 410. Sound field 476 emanating from ultrasound transducer 475 is focused on Dorsal Anterior Cingulate Gyms (DACG) 430. Sound field 481 emanating from ultrasound transducer 480 is focused on Insula 420. All of the ultrasound transducers are mounted on frame 460 with the ultrasound conducted through conductive gel layer 462, conductive medium 450, and conductive gel layer 402 that provides the interface to head 400.

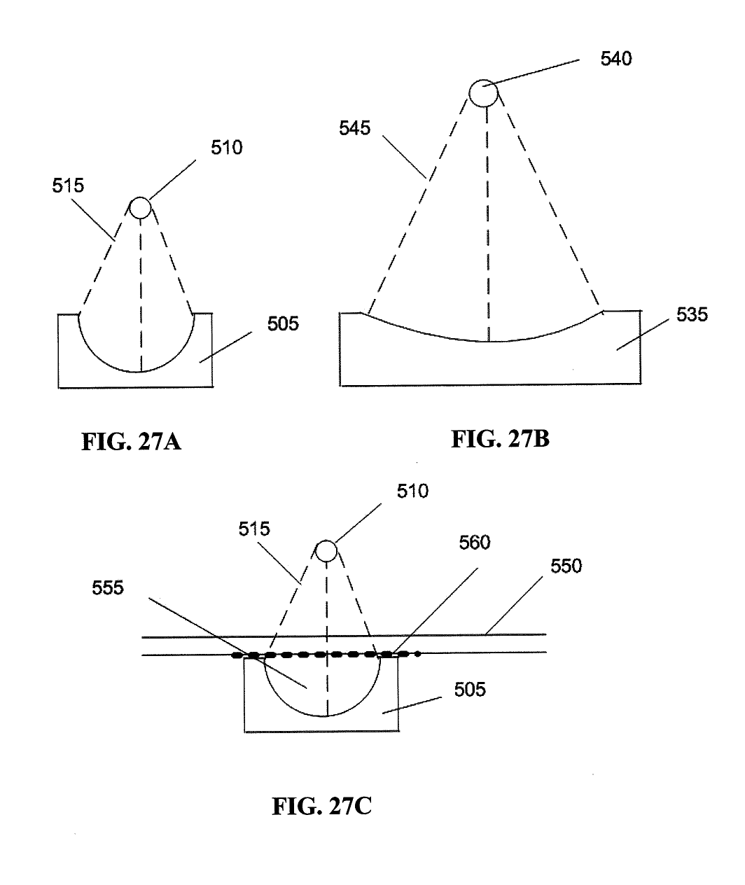

FIGS. 27A-27C demonstrates two ultrasound transducer arrays with different radii. The array with the shorter focal length in FIG. 27A has transducer array 505 focusing sound field 505 at target 510. In FIG. 27B, the array with the longer focal length because of the larger radius has transducer array 535 focusing sound field 545 at target 540. In order to work, there must be a medium between the transducer array and the head to conduct the sound. In FIG. 27C shows the transducer array 505 of FIG. 27A with sound field 515 focused on target 510 with sound conduction media in place between array 505 and head 550. The conduction mechanism consists of hemispheric conduction medium 555 and conducting-gel layer 560 providing the physical interface to head 550.

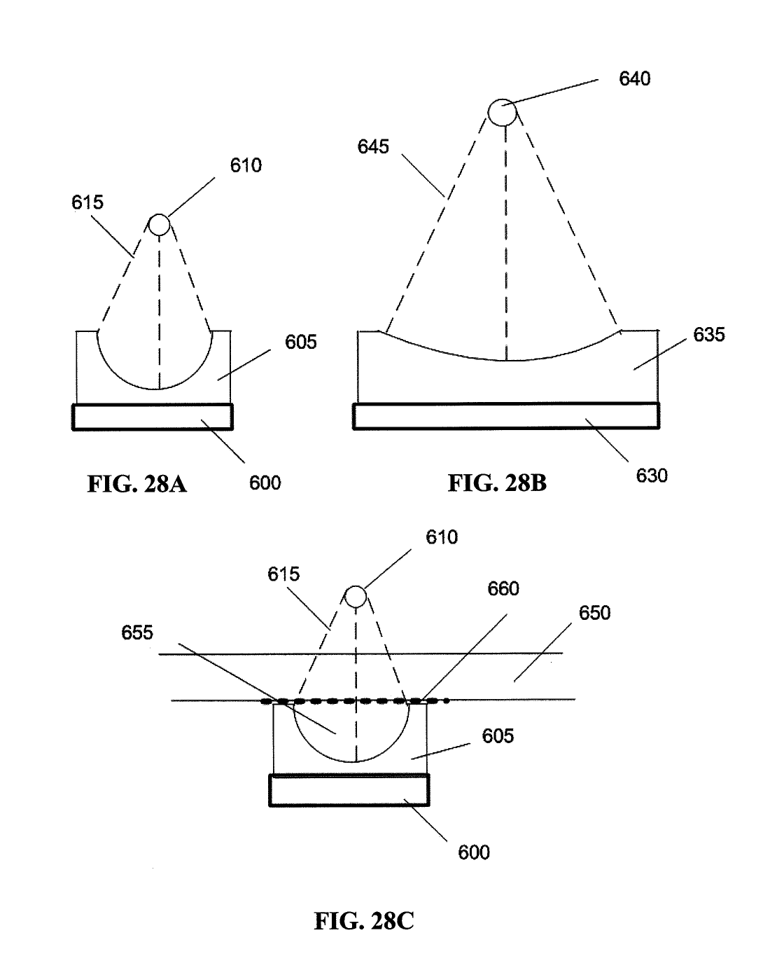

FIGS. 28A-28C demonstrate an embodiment where a flat transducer array is used in conjunction with interchangeable lenses. The configurations are the same as those in FIGS. 27A-27C with the curved transducer array replaced by a combination of a flat transducer array and a curved lens. In FIG. 28A, flat transducer array 600 has its sound field focused by curved lens 605 with sound field 615 focused on target 610. In FIG. 28B, flat transducer array 630 has its sound field focused by curved lens 635 with sound field 645 focused on target 640. FIG. 28C shows the transducer array 600 with lens 605 of FIG. 28A with sound field 615 focused on target 610 with sound conduction media in place between lens 605 and head 650. The conduction mechanism consists of hemispheric conduction medium 655 and conducting-gel layer 660 providing the physical interface to head 650. These lenses can be bonded to flat transducers or non-permanently affixed. With fixed transducer radii configured to not require beam steering, simpler driving electronics can be used. In some embodiments, a portion of a hemisphere can be used as opposed to a full hemisphere, but in these cases, the power required to achieve a given depth will typically be larger. Different focal depths can be achieved by alterations and different field shapes can be achieved by different array transducer shapes (e.g., curved elongated as opposed to flat linear, square, or hemispheric).

An important reason to use the flat transducer with either a fixed or interchangeable lens is that a simple fixed or variable function generator or equivalent can be used (cost in hundreds to low thousands of dollars) as opposed a beam-steering variable amplitude and phase generator (costs in the tens of thousands of dollars). Representative materials for lens construction are metal or epoxy. In an alternative embodiment, a focusable ultrasound lens can be used (G. A. Brock-Fisher and G. G. Vogel, “Multi-Focus Ultrasound Lens”, U.S. Pat. No. 5,738,098).

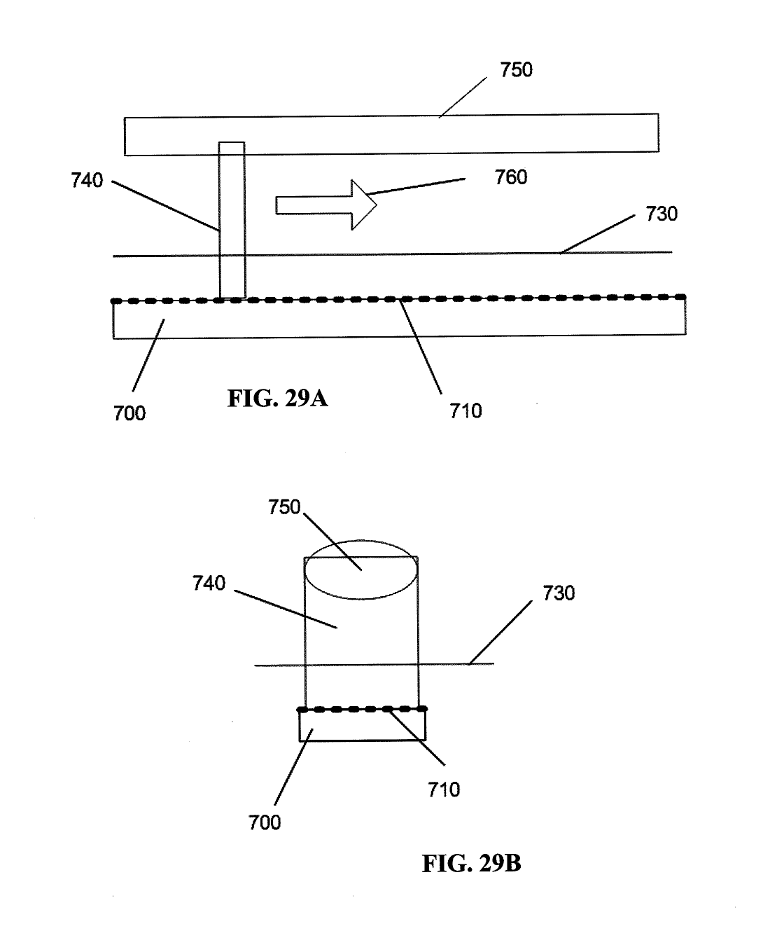

FIGS. 29A-29B show a linear ultrasound phased array with a steered-beam linearly moving field generated by changing the phase/intensity relationships. Beams can also be focused or steered without motion or with non-linear motion. They also can be directed at an angle and not restricted to being aimed perpendicular to the face of the array. FIG. 29A shows a side view and FIG. 29B shows an end view. In FIG. 29A, flat transducer array 700 has its ultrasound conducted by conducting gel layer 710 providing the physical interface to head 730. Sound field 740 moves linearly from left to right as shown by arrow 760 so it moves its focus along target 750. FIG. 29B shows the end view of the configuration looking at the end of flat transducer 700 with conduction of ultrasound to the head 730 provided by conduction layer 710 and sound field 740 focused on target 750. In comparison to FIG. 29A, the sound field 740, which moves, left to right in FIG. 29A moves back into the page in FIG. 29B. In another embodiment, the transducer array is not flat but curved.

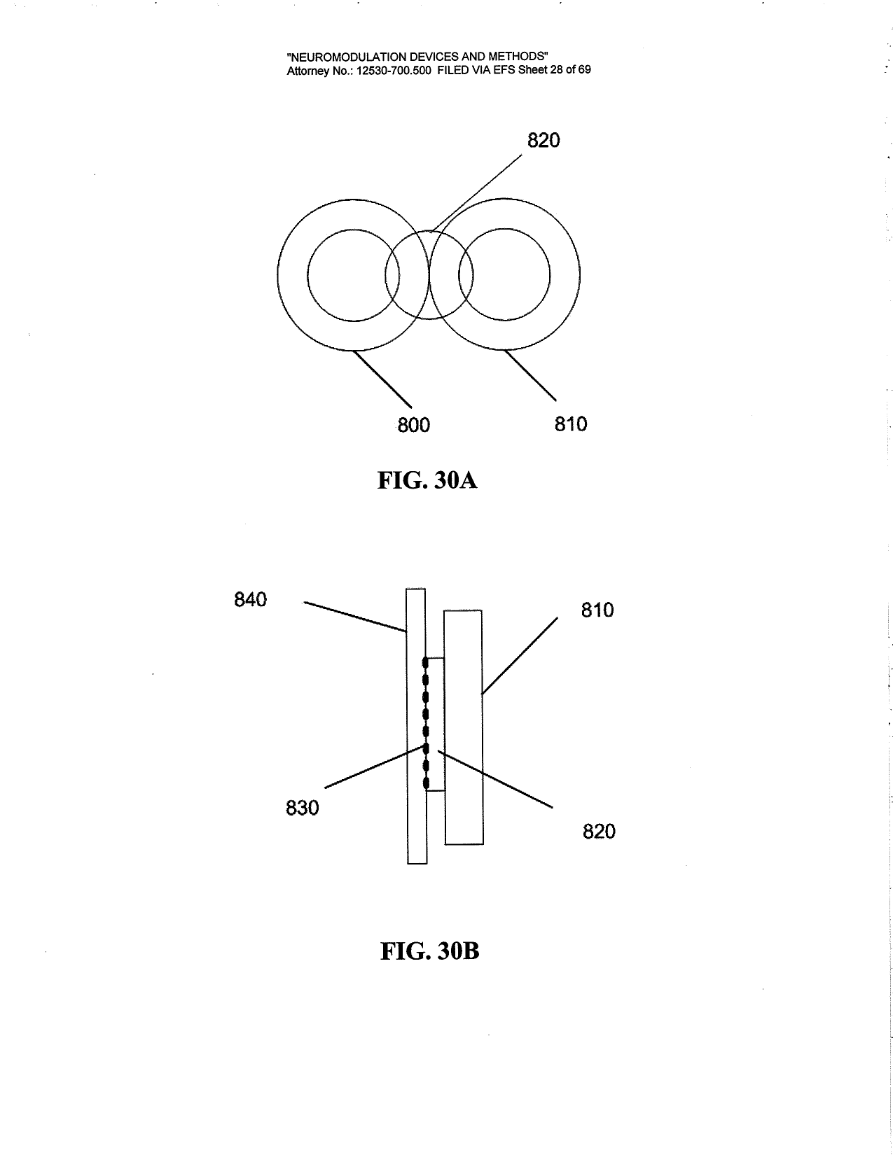

FIGS. 30A-30B demonstrates the combination of an ultrasound transducer with a figure-8 Transcranial Magnetic Stimulation (TMS) Coil in both front and side views. FIG. 30A shows the front view of the TMS electromagnet with its component coils 800 and 810 and the face of ultrasonic transducer. The side view of the configuration with the head 840 included is shown in FIG. 30B with the end view of the TMS electromagnet as to side of coil 810, the side of the ultrasound transducer 820. The ultrasound conduction is provided by conductive-gel layer 830 providing the physical interface between ultrasound transducer array 820, and head 840. MRI-compatible ultrasound generators are available (e.g., from Imasonic) so that the presence of the ultrasound transducer will have minimal impact on the magnetic field generated by the TMS electromagnet.

Any shape of array such as those described above may have its sound field steered or focused. The depth of the point where the ultrasound is focused depends on the setting of the phase and amplitude relationships of the elements of the ultrasound transducer array. The same is true for the lateral position of the focus relative to the central axis of the ultrasound transducer array. An example of directing ultrasound is found in Cain and Frizzell (C. A. Cain and L. A. Frizzell, “Apparatus for Generation and Directing Ultrasound,” U.S. Pat. No. 4,549,533). In another embodiment a viewing hole can be placed in an ultrasound transduction to provide an imaging port. Both Imasonic and Keramos-Etalon supply such configurations.

In other embodiments the transducer can be moved back and forth to cover a long target or vibrate in-and-out or in any direction off the central axis to increase the local effects on neural-structure membranes.

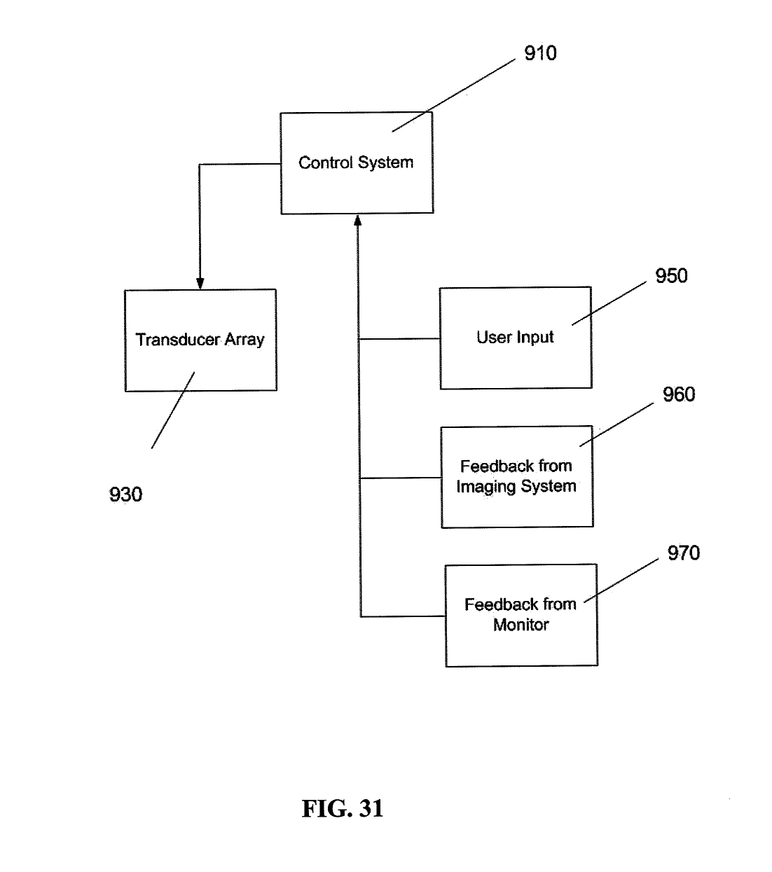

FIG. 31 shows a control block diagram. The positioning and emission characteristics of transducer array 930 are controlled by control system 910 with control input from either user by user input 950 and/or from feedback from imaging system 960 (either automatically or display to the user with actual control through user input 950) and/or feedback from a monitor (sound and/or thermal) 970, and/or the patient 980. Control can be provided, as applicable, for direction of the energy emission, intensity, frequency for up-regulation or down-regulation, firing patterns, and phase/intensity relationships for beam steering and focusing on neural targets. In one embodiment control is also provided for a Transcranial Magnetic Stimulation (TMS) coil as integrated with an ultrasound transducer as shown in FIGS. 30A-30B.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity, which can have benefits, for example, in the early treatment of head trauma or other insults to the brain.

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) or enhances acute effects. In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in some cases only one will targeted (e.g., the right Insula in the case of addiction).

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part V: Treatment Planning for Deep-Brain Neuromodulation

Treatment planning for non-invasive deep brain or superficial neuromodulation using ultrasound and other treatment modalities impacting one or multiple points in a neural circuit to produce acute effects or Long-Term Potentiation (LTP) or Long-Term Depression (LTD) to treat indications such as neurologic and psychiatric conditions. Ultrasound transducers or other energy sources are positioned and the anticipated effects on up-regulation and/or down-regulation of their direction of energy emission, intensity, frequency, firing/timing and phase/intensity relationships mapped onto treatment-planning targets. The maps of treatment-planning targets onto which the mapping occurs can be atlas (e.g., Tailarach Atlas) based or image (e.g., fMRI or PET) based. Imaged-based maps may be representative and applied directly or scaled for the patient or may be specific to the patient.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes, Vagus Nerve Stimulation (VNS), and Sphenopalatine Ganglion Stimulation or other local stimulation).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency. For larger targets, larger spot sizes will be used and, depending on the shape of the targeted area, different shapes of ultrasound fields will be used.

While the description of the invention focuses on ultrasound, treatment planning can be done for therapy using other modalities (e.g., Transcranial Magnetic Stimulation (TMS), transcranial Direct Current Stimulation (tDCS), and/or Deep Brain Stimulation (DBS), Vagus Nerve Stimulation (VNS), Sphenopalatine Ganglion Stimulation and/or other local stimulation using implanted electrodes), and/or future neuromodulation means either individually or in combination.

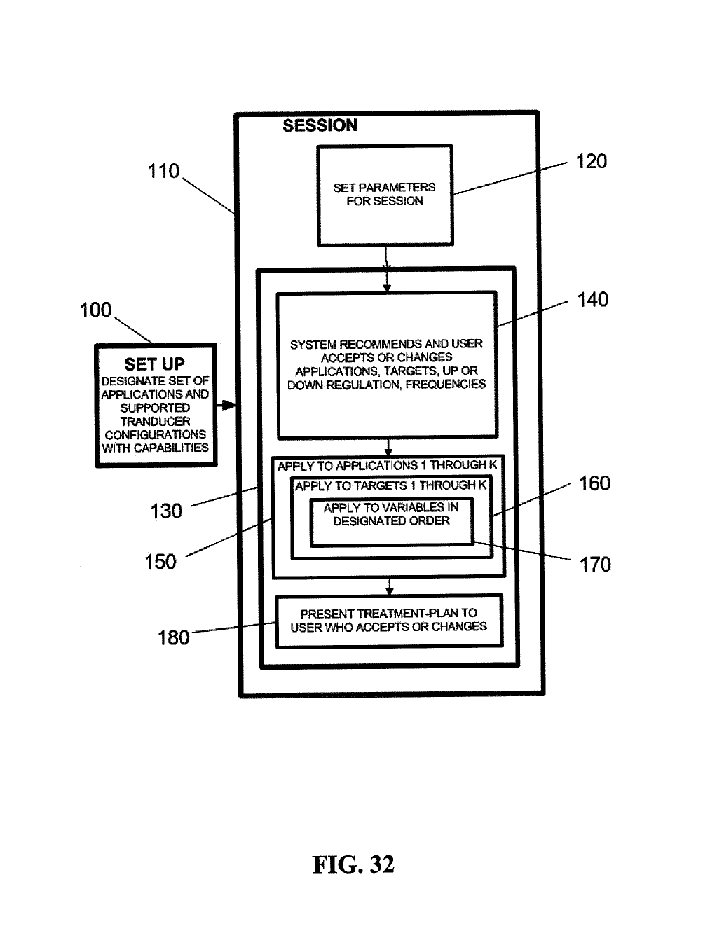

FIG. 32 shows a block diagram of the treatment planning. The set-up 100 designates the set of applications to be considered as well as transducer configurations and capabilities. The session flow 110 involves setting the parameters for the session 120 that is followed by set of activities 130 in which the system recommends and the healthcare-professional user accepts or changes 140 the recommended applications, targets, up- or down-regulation, and frequencies to be used for neuromodulation. Setting of the basic parameters is followed by the application to clinical applications 1 through k 150 which incorporates application to targets 1 through k 160 within which application to variables (from among position, intensity, dynamic sweeps, and firing/timing pattern) 170 in the designated order. In step 180, the resultant treatment plan is presented to the healthcare-professional who accepts or changes the plan. Hitting multiple targets in a neural circuit in a treatment session is an important component of fostering a durable effect through Long-Term Potentiation (LTP) and/or Long-Term Depression (LTD) and is useful for acute effects as well. In addition, this approach can decrease the number of treatment sessions required for a demonstrated effect and to sustain a long-term effect. Follow-up tune-up sessions at one or more later times may be required. The treatment-planning process can be applied to other modalities or a mixture of modalities (e.g., ultrasound used simultaneously with Deep Brain Stimulation or simultaneously or sequentially with Transcranial Magnetic Stimulation). Not all variables be planned for will be same for all modalities and in some cases they may be different than those covered.

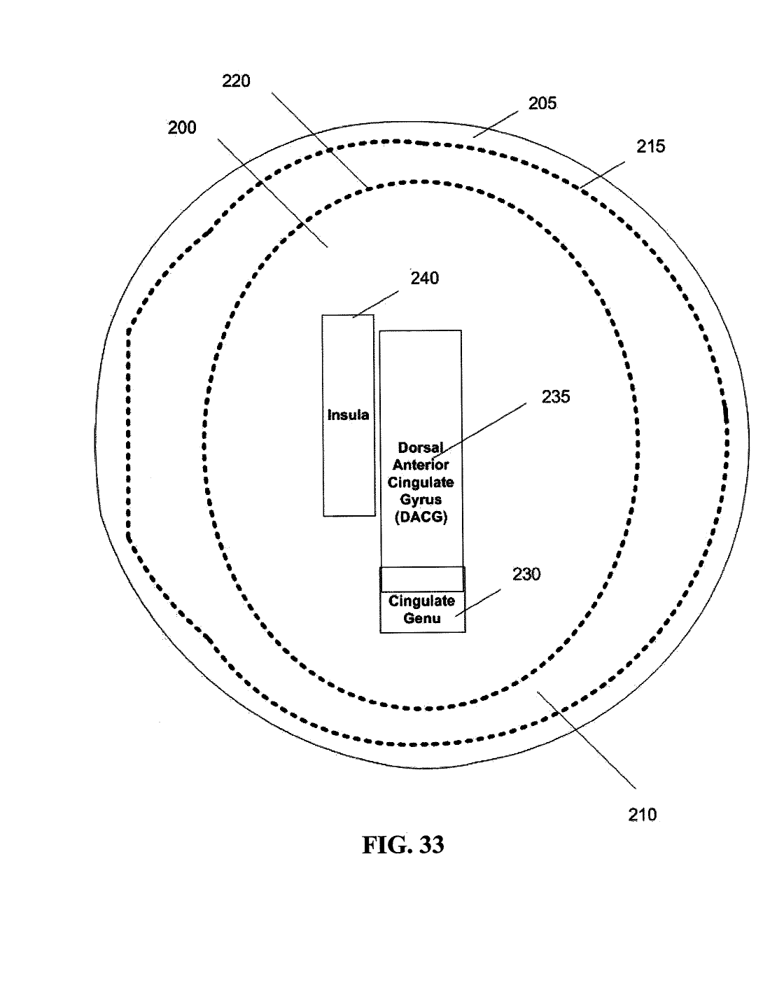

As an example of using the system, in FIG. 33, within patient head 200, three targets related to the processing of pain, the Cingulate Genu 230, Dorsal Anterior Cingulate Gyms (DACG) 235, and Insula 240. These targets, if down regulated through neuromodulation, will decrease the pain perceived by the patient. The physical context of the overall configuration is that the patient head 200 is surrounded by frame 205 on which the ultrasound transducers (not yet attached) will be fixed. Between frame 205 and patient head 200 are interposed the ultrasound-conduction medium 210 (say silicone oil housed within a containment pouch or Dermasol from California Medical Innovations) with the interface between the frame 205 and the ultrasound-conduction medium 210 filled by conduction-gel layer 215 and the interface between ultrasound-conduction medium 210 and patient head 200 filled by conduction-gel layer 220. For the ultrasound to be effectively transmitted to and through the skull and to brain targets, coupling must be put into place. This is only one configuration. In the other embodiments, the ultrasound-conduction medium and the gel layers do not have to completely surround the head, but only need be placed where the ultrasound transducers are located.

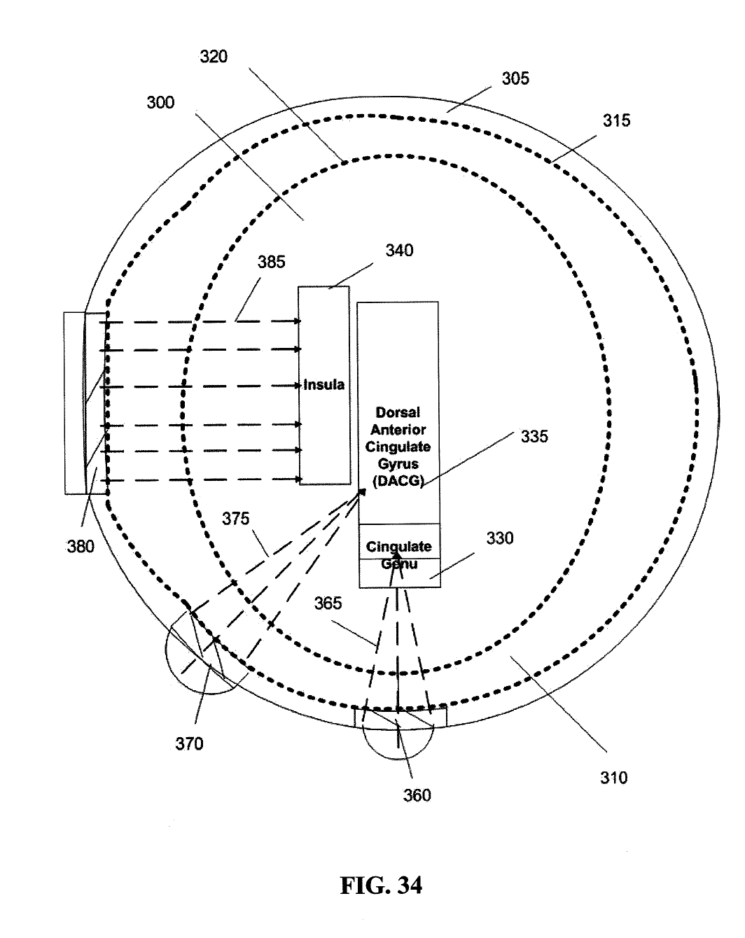

After the treatment planning of FIG. 32 is applied, the graphic as shown in FIG. 34 is displayed so the healthcare-professional can both understand the plan and place the transducers on the frame. Vertical location would be given as well (not shown) as well as saggital and coronal views displayed (not shown). In FIG. 34, patient head 300 is again surrounded by a frame 305 with interposed elements ultrasound-transmission-gel layer 320, ultrasound-transmission medium 310, and ultrasound-transmission-gel layer 315. The display shows the positioning of ultrasound transducer 360 aimed at the Cingulate Genu target 330 and the planned ultrasound field 365. In like manner, the display shows the positioning of ultrasound transducer 370 aimed at the Dorsal Anterior Cingulate Gyms (DACG) target 335 with the planned ultrasound field 375. This display also shows the positioning of ultrasound transducer 380 aimed at the Insula target 340 with the planned ultrasound field 385.

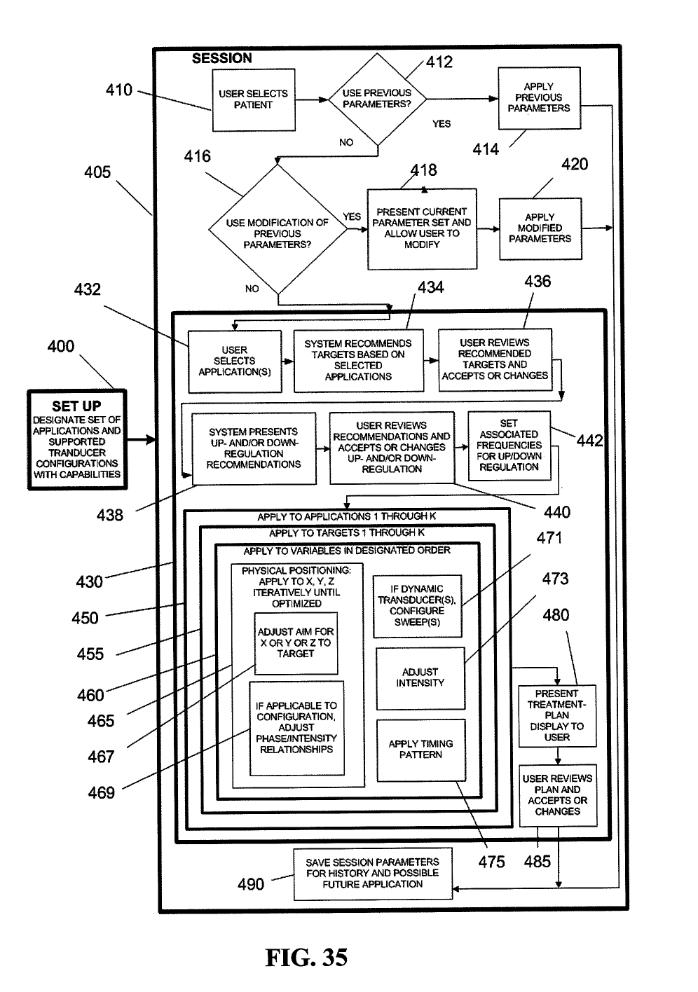

The treatment-planning process covered in FIG. 32 is shown in FIG. 35. Set up 400 includes designation of the set of applications and supported transducer configurations. Session 405 begins with step 410 where the healthcare-professional user selects the patient, which is followed by decision-step 412 as to whether or not previous parameters are to be used. If the response is yes then step 414 is executed, the application of previous parameters, after which there is step 490, saving the session parameters for the historical record and possible future application. If the response 412, use of previous parameters, is no, then decision-step 416 is executed, whether there is to be a user-supplied modification of the previous parameters. The response is yes, step 418 presents the current parameter set to the user and allows the user to modify them. Then in step 420, the modified parameters are applied, after which there is step 490, saving the session parameters for the historical record and possible future application. If the response to decision-step 416, whether there is to be a user-supplied modification of the previous parameters is no, then the flow shown in box 430 is followed. In the initial step 432 the health-professional user selects the applications to be used. This is followed by step 434, system recommending the targets based on the selected applications and step 436 where the user reviews the recommended targets and accepts or changes them. Note that for any of the healthcare-professional user's choices that are inconsistent or otherwise cannot be safely applied, the system will notify the user and offer the opportunity for corrections to be made. Step 436 is followed by step 438 in which the system presents the up- and/or down-regulation recommendations and then step 440 in which the user reviews those recommendations and accepts or changes the up- and/or down regulation designations. Down regulation means that the firing rate of the neural target has its firing rate decreased and thus is inhibited and up regulation means that the firing rate of the neural target has its firing rate increased and thus is excited. In the next step 442, the associated frequencies for up- and down-regulation are applied followed by the iterative application of the elements in box 450 in which in the outer loop the process is applied to applications 1 through k. In succeeding inner loop 455, the process is applied iteratively to targets 1 through k and in its succeeding inner loop 460; the process is applied iteratively to variables in the designated order. In step 465, the physical positioning is applied to x, y, and z iteratively until optimized with 467 adjustment of the aim to target, and 469, if applicable to the configuration, adjustment of the phase/intensity relationships for beam steering and/or focus. Step 471, configuring of sweep(s) is executed if there are dynamic transducers. In step 473, the intensity is adjusted, and the firing/timing pattern applied in 475. The ultrasonic firing/timing patterns can be tailored to the response type of a target or the various targets hit within a given neural circuit. In the output of box 450, in step 480, the treatment-plan display is presented to the user followed by step 485 in which the user reviews the plan and accepts or changes it. Again, if the plan is inconsistent or cannot otherwise be safely executed, the system will notify the user and offer the opportunity for corrections to be made. Following acceptance of the treatment plan, there is step 490, saving the session parameters for the historical record and possible future application.

The invention can be applied to individual, simultaneous, or sequential neuromodulation of one or a plurality of targets including, but not limited to NeoCortex, any of the subregions of the Pre-Frontal Cortex, Orbito-Frontal Cortex (OFC), Cingulate Genu, subregions of the Cingulate Gyms, Insula, Amygdala, subregions of the Internal Capsule, Nucleus Accumbens, Hippocampus, Temporal Lobes, Globus Pallidus, subregions of the Thalamus, subregions of the Hypothalamus, Cerebellum, Brainstem, Pons, or any of the tracts between the brain targets.

The invention can be applied to a one or a plurality of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to one or a plurality of cognitive enhancement, hedonic stimulation, enhancement of neural plasticity, improvement in wakefulness, brain mapping, diagnostic applications, and research functions. In addition to stimulation or depression of individual targets, the invention can be used to globally depress neural activity, which can have benefits, for example, in the early treatment of head trauma or other insults to the brain.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part VI: Ultrasound Neuromodulation of the Brain, Nerve Roots, and Peripheral Nerves

Some of the inventions described herein provide methods and systems and methods for ultrasound stimulation of the cortex, nerve roots, and peripheral nerves, and noting or recording muscle responses to clinically assess motor function. In addition, just like Transcranial Magnetic Stimulation, ultrasound neuromodulation can be used to treat depression by stimulating cortex and indirectly impacting deeper centers such as the cingulate gyms through the connections from the superficial cortex to the appropriate deeper centers. Ultrasound can also be used to hit those deeper targets directly. Positron Emission Tomography (PET) or fMRI imaging can be used to detect which areas of the brain are impacted. In addition to any acute positive effect, there will be a long-term “training effect” with Long-Term Depression (LTP) and Long-Term Potentiation (LTD) depending on the central intracranial targets to which the neuromodulated cortex is connected.

Ultrasound stimulation can be applied to the motor cortex, spinal nerve roots, and peripheral nerves and generate Motor Evoked Potentials (MEPs). MEPs elicited by central stimulation will show greater variability than those elicited stimulating spinal nerve roots or peripheral nerves. Stimulation results can be recorded using evoked potential or electromyographic (EMG) instrumentation. Muscle Action Potentials (MAPs) can be evaluated without averaging while Nerve Action Potentials (NAPS) may need to be averaged because of the lower amplitude. Such measurements can be used to measure Peripheral Nerve Conduction Velocity (PNCV). Pre-activation of the target muscle by having the patient contract the target muscle can reduce the threshold of stimulation, increase response amplitude, and reduce response latency. Another test is Central Motor Conduction Time (CMCT), which measures the conduction time from the motor cortex to the target muscle. Different muscles are mapped to different nerve routes (e.g., Abductor Digiti Minimi (ADM) represents C8 and Tibialis Anterior (TA) represents L4/5). Still another test is Cortico-Motor Threshold. Cortico-motor excitability can be measured using twin-pulse techniques. Sensory nerves can be stimulated as well and Sensory Evoked Potentials (SEPs) recorded such as stimulation at the wrist (say the median nerve) and recording more peripherally (say over the index finger). Examples of applications include coma evaluation (diagnostic and predictive), epilepsy (measure effects of anti-epileptic drugs), drug effects on cortico-motor excitability for drug monitoring, facial-nerve functionality (including Bell's Palsy), evaluation of dystonia, evaluation of Tourette's Syndrome, exploration of Huntington's Disease abnormalities, monitoring and evaluating motor-neuron diseases such as amyotrophic lateral sclerosis, study of myoclonus, study of postural tremors, monitoring and evaluation of multiple sclerosis, evaluation of movement disorders with abnormalities unrelated to pyramidal-tract lesions, and evaluation of Parkinson's Disease. As evident by the conditions that can be studied with the various functions, neurophysiologic research in a number of areas is supported. Other applications include monitoring in the operating room (say before, during, and after spinal cord surgery). Cortical stimulation can provide relief for conditions such as depression, bipolar disorder, pain, schizophrenia, post-traumatic stress disorder (PTSD), and Tourette syndrome. Another application is stimulation of the phrenic nerve for the evaluation of respiratory muscle function. Clinical neurophysiologic research such as the study of plasticity.

When TMS is applied to the left dorsal lateral prefrontal cortex and depression is treated ‘indirectly” (e.g., at 10 Hz, although other rates such as 1, 5, 15, and 20 Hz have been used successfully as well) due to connections to one or more deeper structures such as the cingulate and the insula as demonstrated by imaging. The same is true for ultrasound stimulation.

A benefit of ultrasound stimulation. over Transcranial Magnetic Stimulation is safety in that the sound produced is less with a lower chance of auditory damage. Ironically, TMS produces a clicking sound in the auditory range because of deformation of the electromagnet coils during pulsing, while ultrasound stimulation is significantly above the auditory range.

The acoustic frequency (e.g., typically in that range of 0.3 MHz to 0.8 MHz or above whether cranial bone is to be penetrated or not) is gated at the lower rate to impact the neuronal structures as desired. A rate of 300 Hz (or lower) causes inhibition (down-regulation) (depending on condition and patient). A rate in the range of 500 Hz to 5 MHz causes excitation (up-regulation)). Power is generally applied at a level less than 60 mW/cm2. Ultrasound pulses may be monophasic or biphasic, the choice made based on the specific patient and condition. Ultrasound stimulators are well known and widely available.

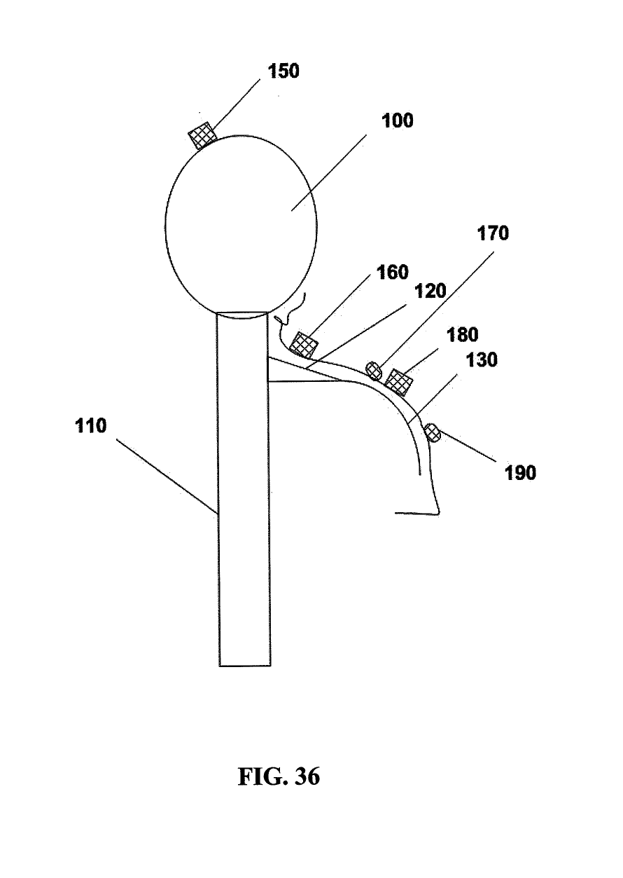

FIG. 36 illustrates placement of ultrasound stimulators EMG and sensors related to head 100, spinal cord 110, nerve root 120, and peripheral nerve 130. Ultrasound transducer 150 is directed at superficial cortex (say motor cortex). For any ultrasound transducer position, ultrasound transmission medium (e.g., silicone oil in a containment pouch) and/or an ultrasonic gel layer. When the ultrasound transducer is pulsed [typically tone burst durations of (but not limited to) 25 to 500 μsec, the conduction time to the sensor at nerve root 170 and/or associated muscles further in the periphery 190. Alternatively ultrasound transducer 160 may be positioned at a nerve root 120 and the conduction time to the electromyography sensor 190 measured. Further, an ultrasound transducer 180 may be positioned over peripheral nerve 130 and the conduction tine to electromyography sensor 190 measured.

Cortical excitability can be measured using single pulses to determine the motor threshold (defined as the lowest intensity that evokes MEPs for one-half of the stimulations. In addition, such single pulses delivered at a level above threshold can be used to study the suppression of voluntarily contracted muscle EMG activity following an induced MEP.

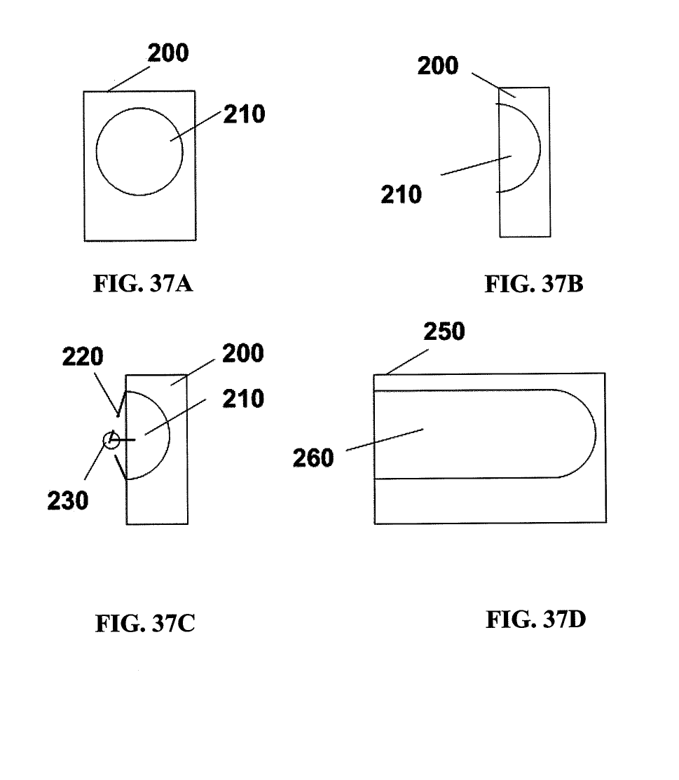

Ultrasound transducer 200 with ultrasound-conduction-medium insert 210 are shown in front view in FIG. 37A and the side view in FIG. 37B. FIG. 37C again shows a side view of ultrasound transducer 200 and ultrasound-conduction-medium insert 210 with ultrasound field 220 focused on the target nerve bundle target 230. Depending on the focal length of the ultrasound field, the length of the ultrasound transducer assembly can be increased with a corresponding increase in the length of ultrasound-conduction-medium insert. For example, FIG. 37D shows a longer ultrasound transducer body 250 and longer ultrasound-conduction-medium insert 260. The focus of ultrasound transducer 200 can be purely through the physical configuration of its transducer array (e.g., the radius of the array) or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Keramos-Etalon can supply a 1-inch diameter ultrasound transducer and a focal length of 2 inches, which with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well. Other embodiments have mechanisms for focus of the ultrasound including fixed ultrasound array, flat ultrasound array with lens, non-flat ultrasound array with lens, flat ultrasound array with controlled phase and intensity relationships, and ultrasound non-flat array with controlled phase and intensity relationship. Ultrasound conduction medium will be required to fill the space. Examples of sound-conduction media are Dermasol from California Medical Innovations or silicone oil in a containment pouch. If patient sees impact, he or she can move transducer (or ask the operator to do so) in the X-Y direction (Z direction is along the length of transducer holder and could be adjusted as well).

Transducer arrays of the type 200 may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07-Feb. 8, 2002), typically with numbers of ultrasound transducers of 300 or more. Keramos-Etalon in the U.S. is another custom-transducer supplier. The design of the individual array elements and power applied will determine whether the ultrasound is high intensity or low intensity (or medium intensity) and because the ultrasound transducers are custom, any mechanical or electrical changes can be made, if and as required. Blatek in the U.S. also supplies such configurations.

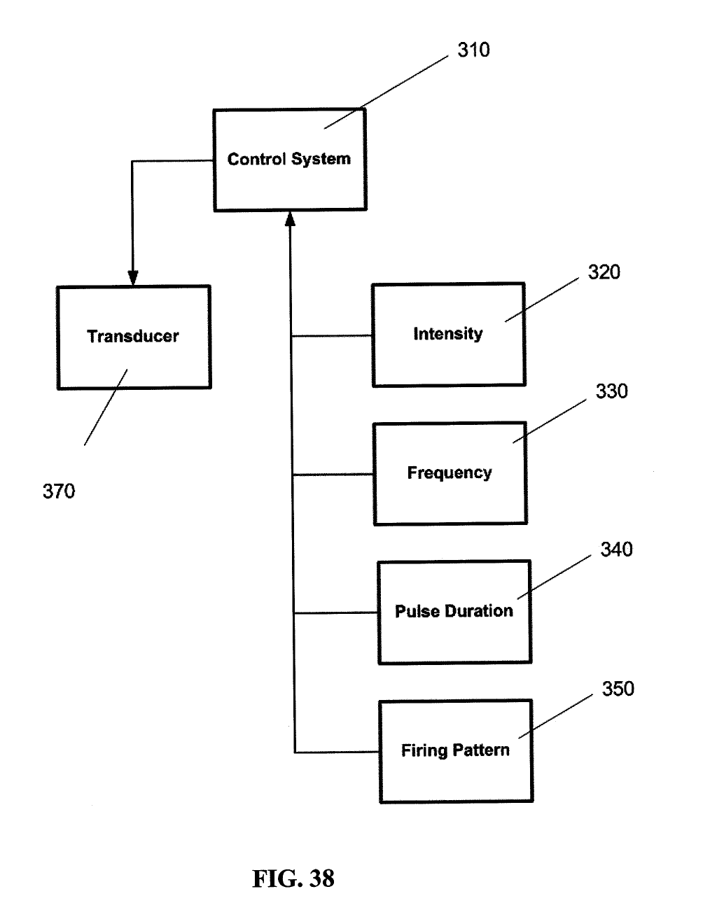

FIG. 38 illustrates the control circuit. Control System 310 receives its input from Intensity setting 320, Frequency setting 330, Pulse-Duration setting 340, and Firing-Pattern setting 350. Control System 310 then provides output to drive Ultrasound Transducer 370 and thus deliver the neuromodulation.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part VII: Ultrasound Macro-Pulse and Micro-Pulse Shapes for Neuromodulation

It is one purpose of some of the inventions described herein to provide methods and systems and methods for non-invasive ultrasound stimulation of neural structures, whether the central nervous systems (such as the brain), nerve roots, or peripheral nerves using macro- and micro-pulse shaping. Ultrasound neuromodulation can be used to treat a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. It can be also applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity that can have benefits, for example, in the early treatment of head trauma or other insults to the brain. Positron Emission Tomography (PET) or fMRI imaging can be used to detect which areas of the brain are impacted. In addition to any acute positive effect, there will be a long-term “training effect” with Long-Term Depression (LTP) and Long-Term Potentiation (LTD) depending on the central intracranial targets to which the neuromodulated cortex is connected. In addition, the effect on a readily observable function such as stimulation of the palm and assessing the impact on finger movements can be done and the effect of changing of the macro-pulse and/or micro-pulse characteristics observed.

The acoustic frequency (e.g., typically in the range of 0.3 MHz to 0.8 MHz or above whether cranial bone is to be penetrated or not) is gated at the lower rate to impact the neuronal structures as desired. A rate of 300 Hz (or lower) causes inhibition (down-regulation) (depending on condition and patient). A rate in the range of 500 Hz to 5 MHz causes excitation (up-regulation)). Power is generally applied at a level less than 60 mW/cm2. Ultrasound pulses may be monophasic or biphasic, the choice made based on the specific patient and condition. Ultrasound stimulators are well known and widely available.

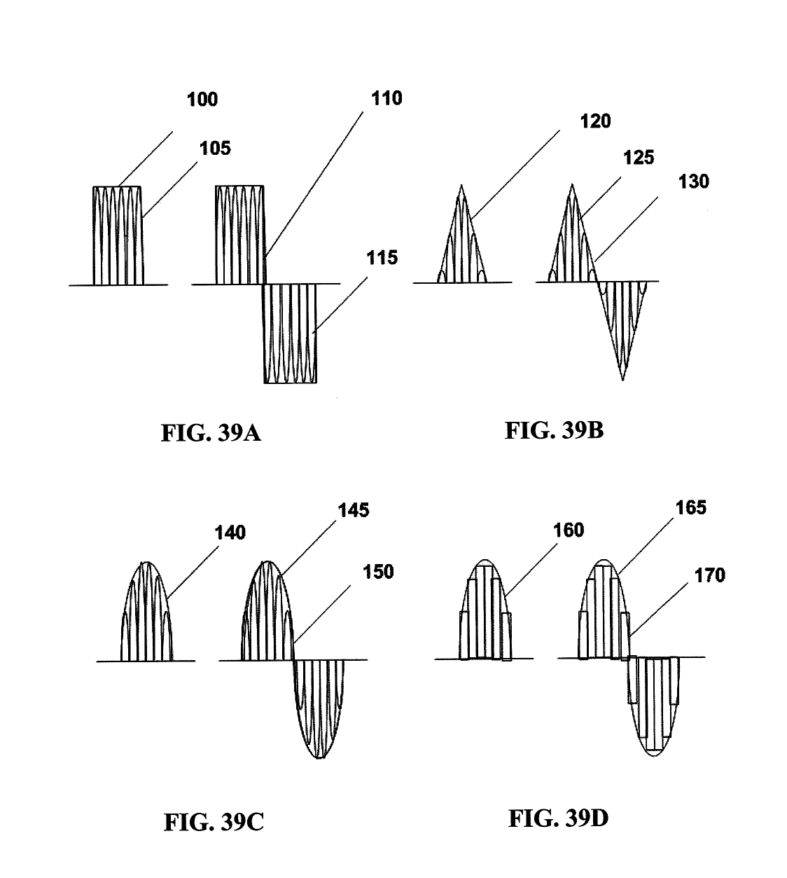

FIGS. 39A-39D demonstrate macro-pulse shaping defined as the overall shape of the pulse burst. The individual pulses making up the macro-pulse shapes are the micro-pulse shapes. FIG. 39A shows monophasic square-wave macro-pulse 100 and biphasic square-wave macro-pulse 110 made up of sine-wave micro-pulses 105. FIG. 39B illustrates monophasic triangular macro-pulse 120 and biphasic triangular macro-pulse 130 made up of sine-wave micro-pulses 125. FIG. 39C illustrates monophasic sinusoidal macro-pulse 140 and biphasic sinusoidal macro-pulse 150 made up of sine-wave micro-pulses 145. FIG. 39D illustrates monophasic sinusoidal macro-pulse 160 and biphasic sinusoidal macro-pulse 170, in this case made up of square-wave micro-pulses 165.

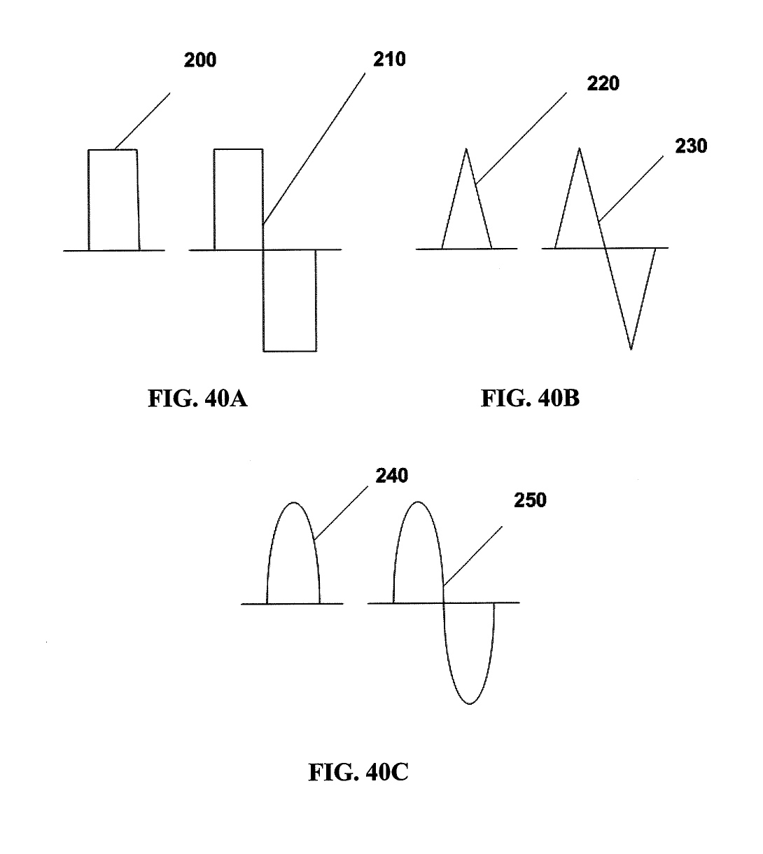

FIGS. 40A-40C show the micro-pulse shapes that can make up the macro-pulse shapes. FIG. 40A illustrates monophasic square-wave pulse 200 and biphasic square-wave pulse 210. FIG. 40B illustrates monophasic triangular pulse 220 and biphasic triangular pulse 230. FIG. 40C illustrates monophasic sinusoidal pulse 240 and biphasic sinusoidal pulse 250.

Other embodiments can be used with different shapes including those created by signal generators capable of producing arbitrary shapes. The pulse shape can affect the effectiveness of the stimulation and that may vary by ultrasound target. Pulse lengths can be with initial rise times on the 100 microseconds with total pulse length of hundreds of microseconds to one millisecond or more. Another facet of the stimulation is the shape of the pulse and whether the pulse is monophasic or biphasic. As to repetition rate, rates on the order of 1 Hz or less typically down-regulate and several Hz. and above up-regulate.

Which macro-pulse and micro-pulse shapes are most effect depends on the target. This can be assessed either by functional results (e.g., doing motor cortex stimulation and seeing which macro- and micro-pulse shape combination causes the greatest motor response) or by imaging (e.g., PET of fMRI) results. Alternatively, the effectiveness of macro-pulse or micro-pulse neuromodulation can be judged by stimulation the palm and assessing the impact of finger movements.

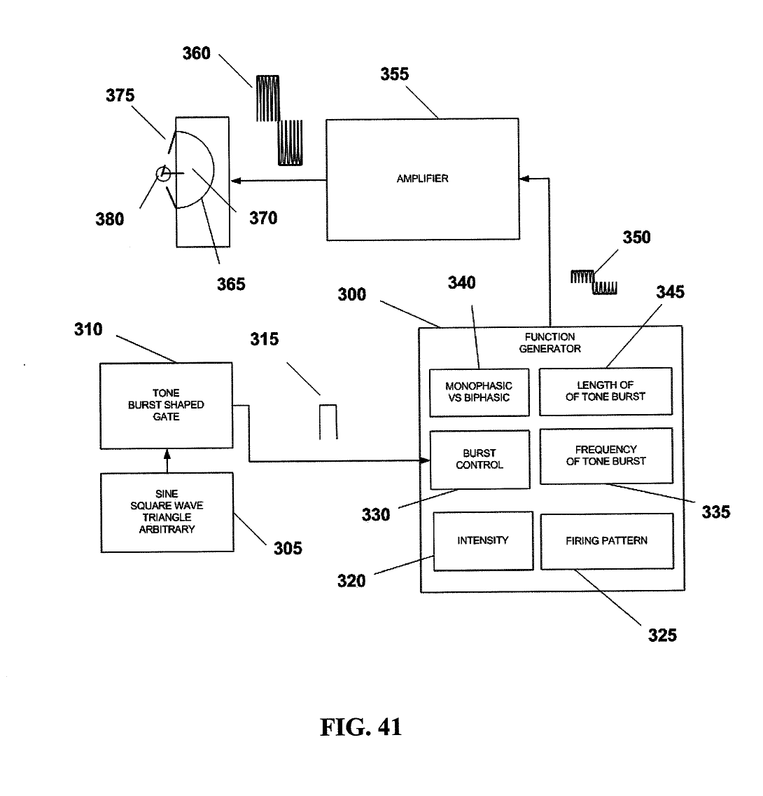

The system for generating the macro- and micro-pulse shapes is shown in FIG. 41. The macro-pulse shape (in this case a square wave) is generated by tone-burst-shaped gate 310 driven by shape control (sine, square-wave, triangle, or arbitrary) 305. The output of tone-burst-shaped gate 310 is 315 and provides input to burst control 330 of function generator 300. The other elements controlled are frequency-of-tone-burst control 335, intensity control 320, firing-pattern control 325, monophasic versus biphasic control 340, length-of-tone-burst control 345. The ultrasound transducer is pulsed with tone burst durations of (but not limited to) 25 to 500 μsec. The resulting output (in this case square-wave macro-pulse made up of sine-wave micro-pulses) 350 provides input to amplifier (for example AB linear) 355 that provides the increased power as output, shown as increased amplitude pulses 360. This drives ultrasound transducer 365 with ultrasound conduction medium 370 generating focused ultrasound field 375 aimed at neural target 380. For any ultrasound transducer position, ultrasound transmission medium (e.g., Dermasol from California Medical Innovations or silicone oil in a containment pouch) and/or an ultrasonic gel layer. Depending on the focal length of the ultrasound field, the length of the ultrasound transducer assembly can be increased with a corresponding increase in the length of ultrasound-conduction-medium insert. The focus of ultrasound transducer 365 can be purely through the physical configuration of its transducer array (e.g., the radius of the array) with an optional lens or by focus or change of focus by control of phase and intensity relationships among the array elements. In an alternative embodiment, the ultrasonic array is flat or other fixed but not focusable form and the focus is provided by a lens that is bonded to or not-permanently affixed to the transducer. In a further alternative embodiment, a flat ultrasound transducer is used and the focus is supplied by control of phase and intensity relationships among the transducer array elements.

Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. In an alternative embodiment, focus can be deemphasized or eliminated with a smaller ultrasound transducer diameter with a shorter longitudinal dimension, if desired, as well.

Transducer arrays of the type 365 may also be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle-31/07—Feb. 8, 2002), typically with numbers of ultrasound transducers of 300 or more. The design of the individual array elements and power applied will determine whether the ultrasound is high intensity or low intensity (or medium intensity) and because the ultrasound transducers are custom, any mechanical or electrical changes can be made, if and as required.

In another embodiment the pulses (macro-shaped; micro-shaping is not applicable) of Transcranial Magnetic Stimulation (TMS) are shaped.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part VIII: Patterned Control of Ultrasound for Neuromodulation

Some of the inventions described herein are ultrasound devices using non-intersecting beams or intersecting beams delivering enhanced non-invasive deep brain or superficial deep-brain neuromodulation using patterned stimulation impacting one or a plurality of points in a neural circuit providing for up-regulation or down-regulation of neural targets, as applicable, to produce acute effects (as in the treatment of post-surgical pain) or Long-Term Potentiation (LTP) or Long-Term Depression (LTD). Patterns can be applied to multiple beams that intersect to stimulate a single target. One reason for using such intersecting beams is to divide the applied power into multiple components so that the power can be utilized to adequately neuromodulate the intended target without over-stimulating the tissues between the ultrasound transducers and the target and causing undesirable side effects such as seizures.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of sound transducers of 300 or more. Blatek and Keramos-Etalon in the U.S. are other custom-transducer suppliers. The power applied will determine whether the ultrasound is high intensity or low intensity (or medium intensity) and because the sound transducers are custom, any mechanical or electrical changes can be made, if and as required.

The locations and orientations of the transducers and their stimulation patterns in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. The ultrasonic firing patterns can be tailored to the response type of a target or the various targets hit within a given neural circuit.

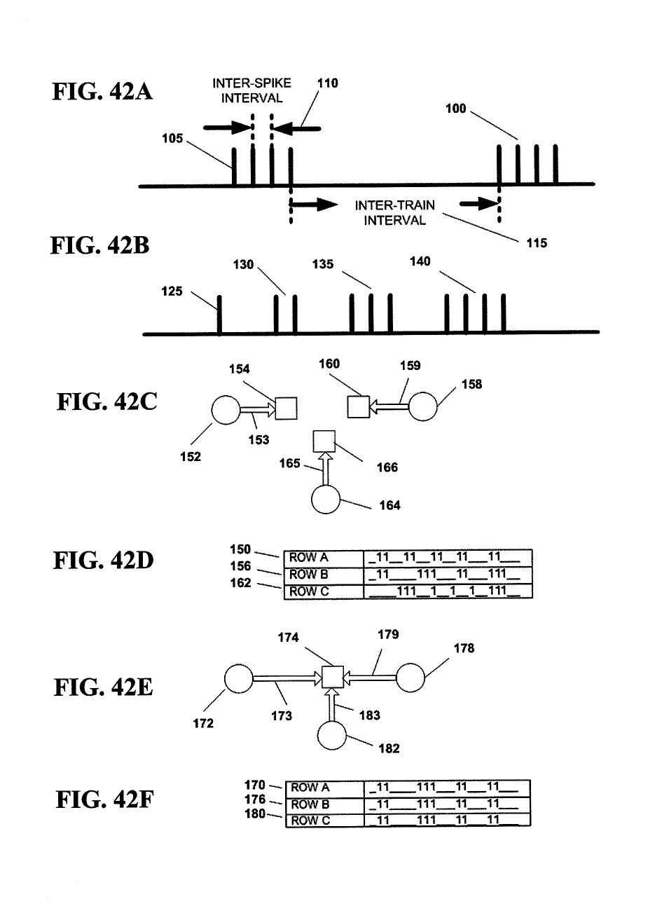

FIGS. 42A-42F illustrate examples of patterns. In FIG. 42A, Pulse trains 100 are composed of one or a plurality of sets of pulses (e.g., singletons, pairs, triplets, etc.) made up of individual pulses 105 with inter-spike intervals 110 with the trains separated by inter-pulse-train intervals 115. If the set of inter-pulse intervals 130 is of length zero, then the train is continuous. FIG. 42B illustrates examples of an individual pulse singlet 125 as well as pulse sets pulse pair 130, pulse triplet 135, and pulse quadruplet 140. The elements of a train may the same or they may vary. For example, a pair of pulses may alternate with a triplet of pulses and/or the inter-pulse-train intervals may vary. Patterns applied may be either fixed or random. Sample patterns include pairs, triplets, or other multiplicates, theta burst stimulations, alternating simple patterns (e.g., alternating pairs with triplets), changing frequencies during stimulations (e.g., for a singlet ramping up the stimulation frequency from 5 Hz. to 20 Hz. over a period of 15 stimulations and then ramping down the stimulation from 20 Hz to 5 Hz. in the next 15 stimulations where the frequencies increase and decrease can be linear or non-linear), and others. Variable or fixed patterns can apply to individual targets or among targets. An example of another pattern is Theta-Burst Stimulation (TBS) that consists of short bursts (e.g., 3) of high-frequency pulses impulses repeated at 5 Hz (the frequency of the theta rhythm in the EEG). In some cases the pattern applied to a given neural target or neural circuit may constitute a natural rhythm for that target or circuit and may even include resonance. Patterns include variations in rate or intensity. The relationship between the applied frequency, timing pattern and applied intensity pattern can be independently varied, dependently varied, independently fixed, and dependently fixed.

FIG. 42C shows a diagram of three ultrasound transducers 152, 158, and 164 with respective ultrasound beams 153, 159, and 165 impacting three targets 154, 160, and 166 supporting patterned stimulation where multiple ultrasonic transducers are each aimed at different targets. Depending on the characteristics of the targets, the stimulation patterns of each transducer in a set of transducers may be the same or different. FIG. 42D illustrates examples of stimulation patterns for the case shown in FIG. 42C. Stimulation-pattern row 150 shows the stimulation pattern for ultrasound transducer 152 aimed at target 154. Stimulation-pattern row 156 shows the stimulation pattern for ultrasound transducer 158 aimed at target 160. Stimulation-pattern row 162 shows the stimulation pattern for ultrasound transducer 164 aimed at target 166.

FIG. 42E shows a diagram of three ultrasound transducers 172, 178, and 182 with respective ultrasound beams 173, 179, 183 impacting common target 174 supporting patterned stimulation where multiple ultrasonic transducers are each aimed at the same target. FIG. 42F illustrates examples of stimulation patterns for the case shown in FIG. 42E. Stimulation-pattern row 170 shows the stimulation pattern for ultrasound transducer 172 aimed at target 174. Stimulation-pattern row 176 shows the stimulation pattern for ultrasound transducer 178 also aimed at target 174. Stimulation-pattern row 180 shows the stimulation pattern for ultrasound transducer 182 again also aimed at target 174. Even when a common target is neuromodulated, adjustment of stimulation parameters may moderate or eliminate a problem with side effects from the neuromodulation.

In the case of synchronous patterns, the same pattern is applied to multiple targets. In the case of asynchronous patterns, different patterns are applied to different targets. In the case of independent patterns when two different patterns are applied to different targets, when one pattern is changed, the other is not changed or not in changed in the same way. If one or a plurality of targets are all up-regulated or all down-regulated or there is a mixture of such regulation, different frequencies can be used to optimize the desired effects on the various targets (e.g., one up-regulation done at 5 Hz. and another at 10 Hz.). Invention includes the concept of having different patterns for each of a pair of bilateral structures. For example, in the treatment of addiction, neuromodulating the Insula involves down regulating the Insula on the right side.

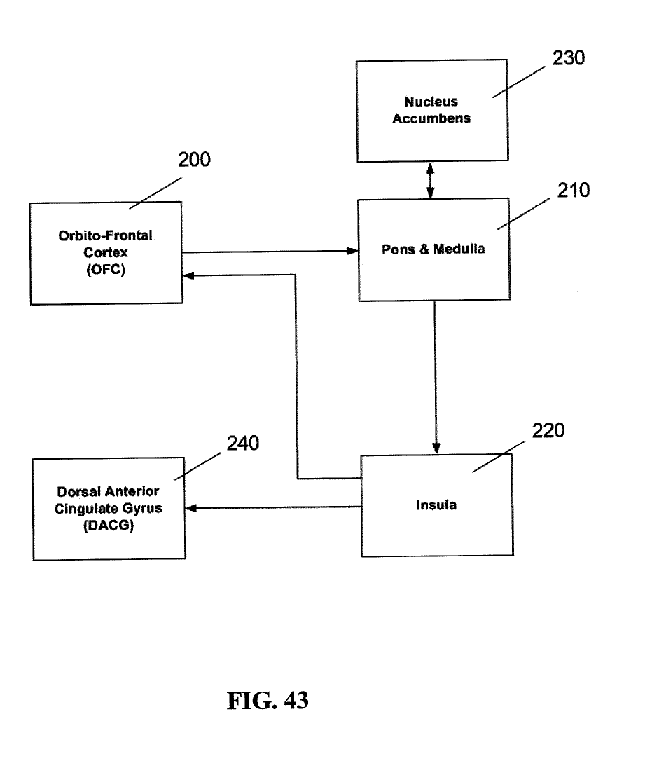

FIG. 43 shows a set of important targets for the treatment of addiction. Five targets are shown, Orbito-Frontal Cortex (OFC) 200, Pons & Medulla 210, Insula 220, Nucleus Accumbens 230, and Dorsal Anterior Cingulate Gyms (DACG) 240.

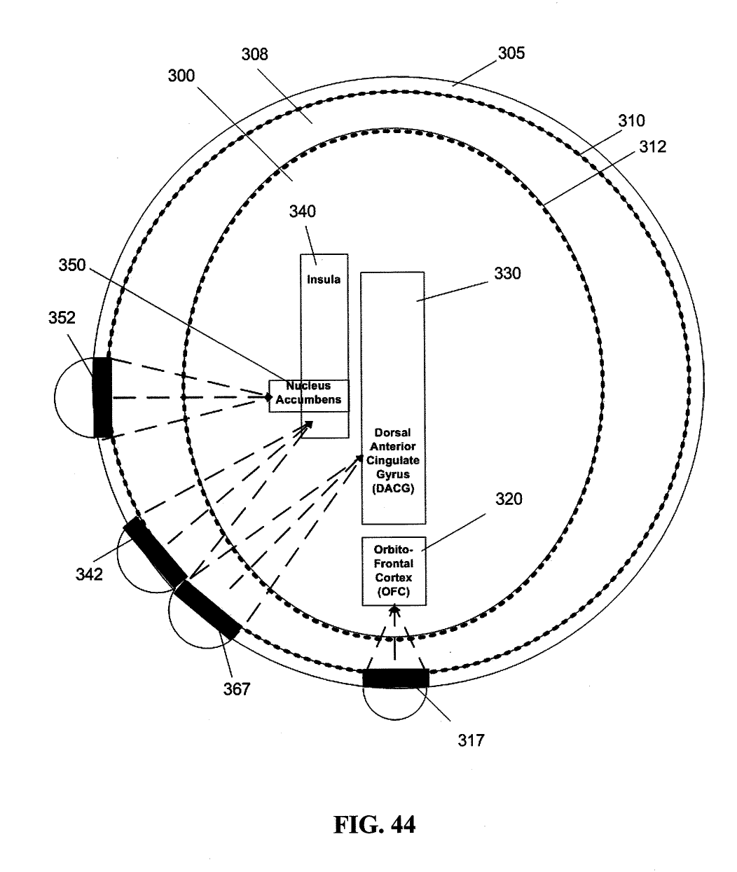

FIG. 44 illustrates within head 300 four targets related to the treatment of addiction from FIG. 43, Orbito-Frontal Cortex (OFC) 320, Dorsal Anterior Cingulate Gyms (DACG) 330, Insula 340, and Nucleus Accumbens 350. Mounted on frame 305 are ultrasound transducers 317 targeting OFC 320, 367 targeting DACG 330, 342 targeting Insula 340, and 352 targeting Nucleus Accumbens 350. Ultrasound transducers 317, 367, 342, and 352 have focused, non-intersecting ultrasound beams. To obtain effective transmission, each of the ultrasound beams is directed through ultrasound conduction medium 308 with layers of ultrasound conduction gel 310 between the ultrasound transducers lens faces and ultrasound conduction gel 312 between the ultrasound conduction medium 308 and that medium and the head 300. Examples of ultrasound conduction media include Dermasol from California Medical Innovations and silicone oil in a containment pouch. In an alternative embodiment instead of a band of ultrasound conduction medium being placed around the head, individual ultrasound conduction media are placed for each ultrasound transducers, again including ultrasound conduction gel layers between the transducer lens face and the conduction medium and also between the ultrasound conduction medium and the head. Pulsed patterns are then used to excite each transducer. To treat addiction, for the four targets being neuromodulated, the Orbito-Frontal Cortex (OFC) and the Nucleus Accumbens are up regulated and the Dorsal Anterior Cingulate Gyms (DACG) and the Insula are down regulated.

One or more targets can be targeted simultaneously or sequentially. Down regulation means that the firing rate of the neural target has its firing rate decreased and thus is inhibited and up regulation means that the firing rate of the neural target has its firing rate increased and thus is excited. The ultrasonic firing/timing patterns can be tailored to the response type of a target or the various targets hit within a given neural circuit.

In another embodiment the ultrasound beams intersect at the targets. This can be useful where one wants to increase the intensity level at a given target, but decrease the intensity of tissue intermediate between the output interface of the ultrasound transducer and the given target. In this invention, two or more beams intersect at a given target with appropriate patterns applied to each of the beams. Use of patterns and/or intersecting ultrasound beams avoids excessive stimulation of nearby structures that need to be protected.

In another embodiment, the neuromodulation of one or a plurality of ultrasound transducers is combined with the neuromodulation from one or a plurality of Transcranial Magnetic Stimulation (TMS) electromagnetic coils. In another embodiment, a viewing hole can be placed in an ultrasound transducer to provide an imaging port. Blatek, Imasonic and Keramos-Etalon can supply such configurations. In another embodiment auditory input can be a neuromodulation modality combined with ultrasound neuromodulation or ultrasound neuromodulation and Transcranial Magnetic Stimulation.

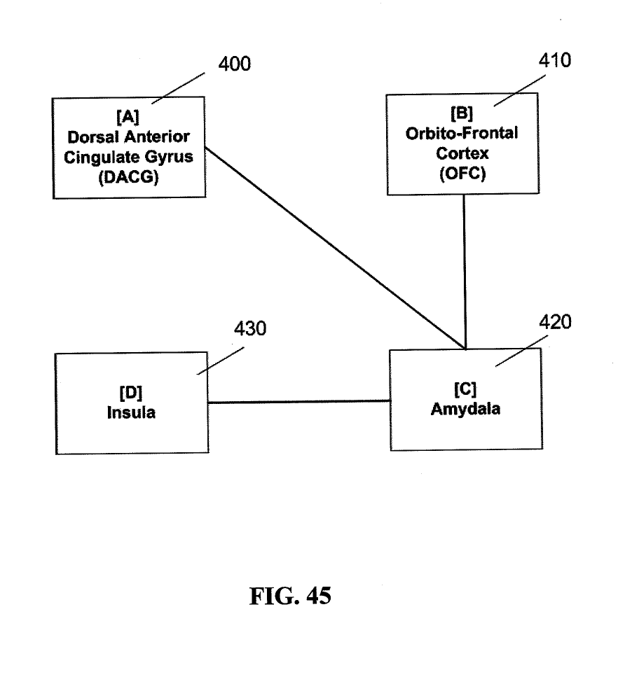

FIG. 45 illustrates the neural circuit representing the case where alternative effects can occur depending on whether the elements of the circuit are either up regulated or down regulated. Note in some cases in a given circuit not all the elements will be all up regulated or down regulated. In FIG. 45, blocks [A] 400, [B] 410, [C] 420, and [D] 430 represent neural elements that can be up regulated or down regulated. In this example, for one clinical effect, all are regulated in the direction to achieve that effect, and for the opposite clinical effect, all are regulated in the opposite direction. As a specific embodiment, for bipolar disorder, [A] 400 represents the Dorsal Anterior Cingulate Gyms (DACG), [B] 410 represents the Orbital-Frontal Cortex (OFC), [C] 420 represents the Amygdala, and [D] 430 represents the Insula. For the condition Bipolar Disorder, if the depressive phase is being treated, the OFC 410, the Amygdala 420, and left-located Insula 430 are down regulated, and the DACG 400 and right-located Insula are up regulated. On the other hand, if the manic phase is being treated, the OFC 410, the Amygdala 420, and left-located Insula 430 are up regulated, and the DACG 400 and right-located Insula 430 are down regulated. In a sense, the circuit is sped up or advanced to treat the depressive phase and slowed down or retarded to treat the manic phase.

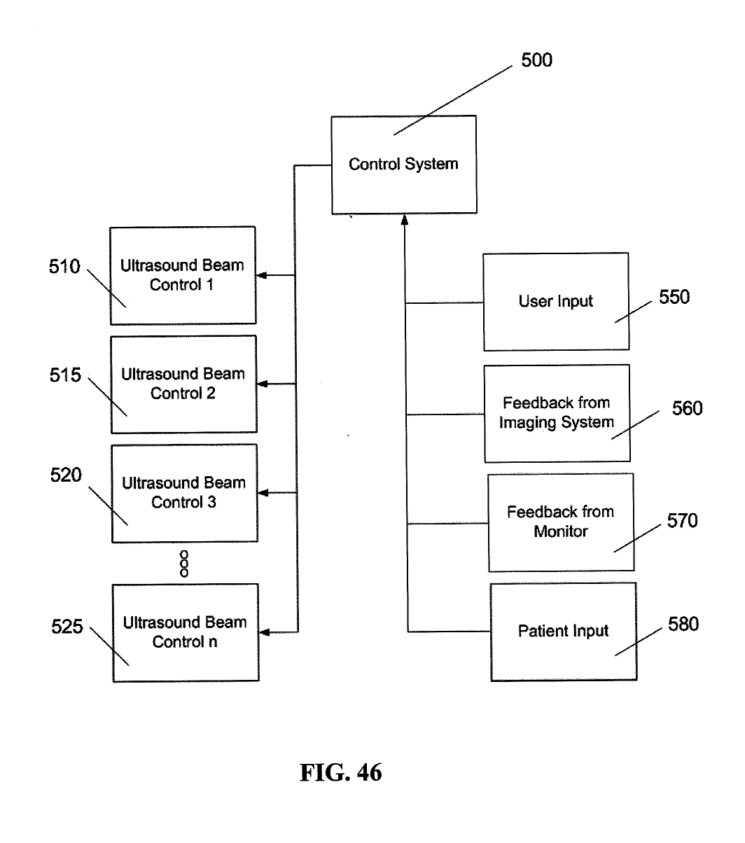

FIG. 46 shows a control block diagram. The frequencies, firing patterns, and intensities for the ultrasonic transducers 510, 515, 520, 525 (and, as applicable, additional ultrasound transducers as indicated by the ellipsis between ultrasound transducers 520 and 525) are controlled by control system 500 with control input from user by user input 550 and/or from feedback from imaging system 560 (either automatically or display to the user with actual control through user input 550), and/or feedback from a functional monitor (one or more of motion, thermal, etc.) 570, and/or the patient 580. If positioning of the ultrasound transducers is included as a control element, then control system 500 will control positioning as well.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity that can have benefits, for example, in the early treatment of head trauma or other insults to the brain.

All of the embodiments above, except those explicitly restricted in configuration to hit a single target, are capable of and usually would be used for targeting multiple targets either simultaneously or sequentially. The invention provides for hitting one or a plurality of targets in a single circuit or a plurality of neural circuits. Hitting multiple targets in a neural circuit in a treatment session is an important component of fostering a durable effect through Long-Term Potentiation (LTP) and/or Long-Term Depression (LTD) or enhances acute effects (e.g., such as treatment of post-surgical pain). In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in some cases unilaterally (e.g., the right Insula in the case of addiction).

The invention allows stimulation adjustments in variables such as, but not limited to, intensity, timing, firing pattern, and frequency, and position to be adjusted so that if a target is in two neuronal circuits the output of the transducer or transducers can be adjusted to get the desired effect and avoid side effects. Position can be adjusted as well. The side effects could occur because for one indication the given target should be up regulated and for the other down regulated. An example is where a target or a nearby target would be down regulated for one indication such as pain, but up-regulated for another indication such as depression.

The invention also covers contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyms (DACG). To counterbalance the down-regulation of the DACG during treatment for pain that negatively impacts the treatment for depression, one would up-regulate the Nucleus Accumbens or Hippocampus that are other targets in the depression neural circuit. A plurality of such applicable targets could be stimulated as well. One set of applied patterns can be applied to a given neural circuit to provide treatment for one condition and an alternative set of applied patterns is applied to the given neural circuit to provide treatment for another condition.

Another applicable scenario is the Nucleus Accumbens that is down regulated to treat addiction, but up regulated to treat depression. To counteract the down-regulation of the Nucleus Accumbens to treat depression but will negatively impact the treatment of depression that would like the Nucleus Accumbens to be up regulated, one would up-regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part IX: Ultrasound-Intersecting Beams for Deep-Brain Neuromodulation

One invention described herein is an ultrasound device using intersecting beams delivering enhanced non-invasive deep brain or superficial deep-brain neuromodulation impacting one or a plurality of points in a neural circuit to produce acute effects (as in the treatment of post-surgical pain) or Long-Term Potentiation (LTP) or Long-Term Depression (LTD) using up-regulation or down-regulation.

The stimulation frequency for inhibition as below 500 Hz (depending on condition and patient). The stimulation frequency for excitation is in the range of 500 Hz to 5 MHz. There is not a sharp border at 500 Hz, however. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). The modulation frequency (superimposed on the carrier frequency of say 0.5 MHz or similar) may be divided into pulses 0.1 to 20 msec. repeated at frequencies of 2 Hz or lower for down regulation and higher than 2 Hz for up regulation) although this will be both patient and condition specific. If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes, optogenetics, radiosurgery, Radio-Frequency (RF)), behavioral therapy, or medications.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound-Seattle-31/07-Feb. 8, 2002), typically with numbers of sound transducers of 300 or more. Blatek and Keramos-Etalon in the U.S. are other custom-transducer suppliers. The power applied will determine whether the ultrasound is high intensity or low intensity (or medium intensity) and because the sound transducers are custom, any mechanical or electrical changes can be made, if and as required.

The locations and orientations of the transducers in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. The ultrasonic firing patterns can be tailored to the response type of a target or the various targets hit within a given neural circuit.

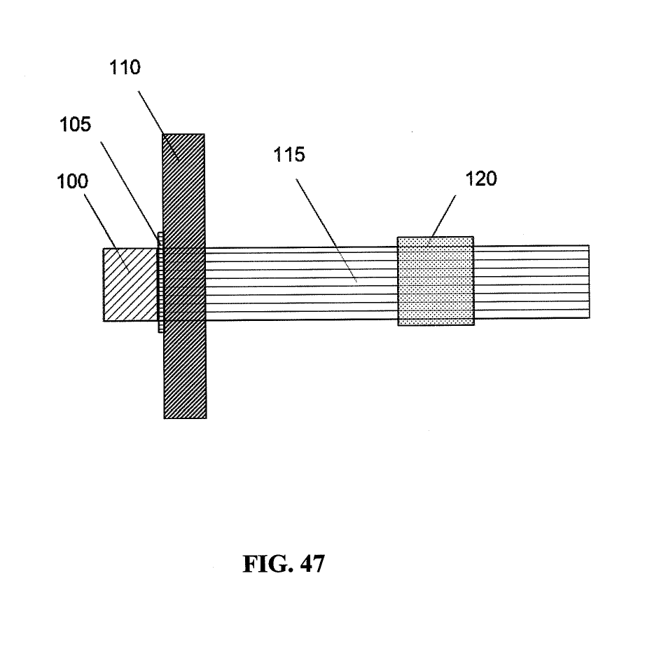

FIG. 47 shows a flat ultrasound transducer producing a parallel beam intersecting a single target. Flat ultrasound transducer 100 produces ultrasound beam 115. To be practical, ultrasound beam 115 passes through skull section 110 with coupling medium 105 interposed between transducer 100 and skull section 110 to support effective transmission. Ultrasound beam 115 hits target 120.

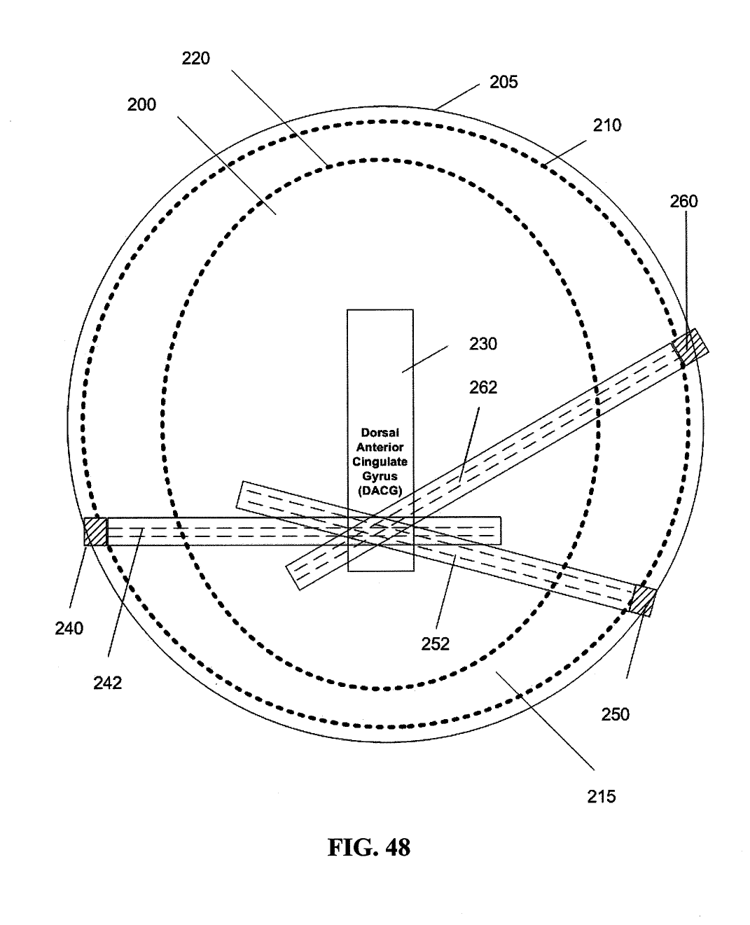

FIG. 48 illustrates head 200 containing target Dorsal Anterior Cingulate Gyms (DACG) 230. Frame 205 holds three ultrasound transducers 240, 250, 260. The beam from each ultrasound transducer passes though an ultrasound-conduction medium 215 with ultrasound-conduction gel interfaces 210 at the transducer face and 220 at the head. Ultrasound transducer 240 generates ultrasound beam 242, ultrasound transducer 250 generates ultrasound beam 252, and ultrasound transducer 260 generates ultrasound beam 262. Ultrasound beams 242, 252, and 262 intersect at Dorsal Anterior Cingulate Gyms target 230 and neuromodulate the DACG. The effects of beams 242, 252, and 262 are additive. Examples of ultrasound conduction media include Dermasol from California Medical Innovations and silicone oil in a containment pouch. Ultrasound-conjunction gel (not shown) can be placed just at the interfaces between any of the ultrasound transducers and the band of ultrasonic-conduction medium 215 and that band and head 200 as long as the beam regions are covered. One or more of the plurality of the ultrasound transducers can also be used with an acoustic lens (not shown). For elongated targets such as the DACG, the intersecting beams can be spread to cover a broader neural region. In addition the width of the ultrasound transducer and thus the width of the beam can be varied.

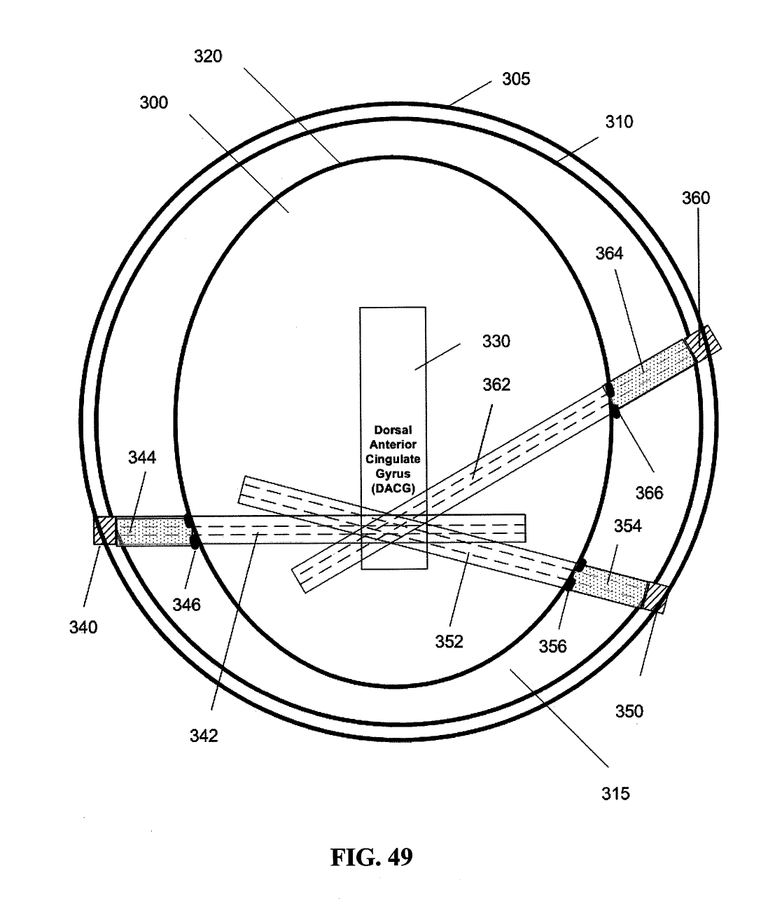

In another embodiment, the ultrasound-conduction medium is not incorporated in a continuous band around the head (215 in FIG. 48), but instead is configured as a single ultrasound conduction medium for each ultrasound transducer. FIG. 49 illustrates head 300 containing target Dorsal Anterior Cingulate Gyms (DACG) 330. Frame 305 holds three ultrasound transducers 340, 350, 360. The beam from each ultrasound transducer passes though individual ultrasound-conduction media. For ultrasound transducer 340, beam 342 passes through ultrasound-conduction medium 344 and then through ultrasound-conduction gel 346 at the interface with head 300. There also can be a layer ultrasound-conduction gel (not shown) at the interface between ultrasound transducer 340 and ultrasound-conduction medium 344. For ultrasound transducer 350, beam 352 passes through ultrasound-conduction medium 354 and then through ultrasound-conduction gel 356 at the interface with head 300. There also can be a layer of ultrasound-conduction gel (not shown) at the interface between ultrasound transducer 350 and ultrasound-conduction medium 354. In like manner, for ultrasound transducer 360, beam 362 passes through ultrasound-conduction medium 364 and then through ultrasound-conduction gel 366 at the interface with head 300. There also can be a layer of ultrasound-conduction gel (not shown) at the interface between ultrasound transducer 360 and ultrasound-conduction medium 364. Ultrasound beams 342, 352, and 362 intersect at Dorsal Anterior Cingulate Gyms target 330 and neuromodulate the DACG. The effects of beams 342, 342, and 362 are additive. Each ultrasound transducer can also be used with an acoustic lens (not shown). For elongated targets such as the DACG, the intersecting beams can be spread to cover a broader neural region. In addition the width of the ultrasound transducer and thus the width of the beam can be varied.

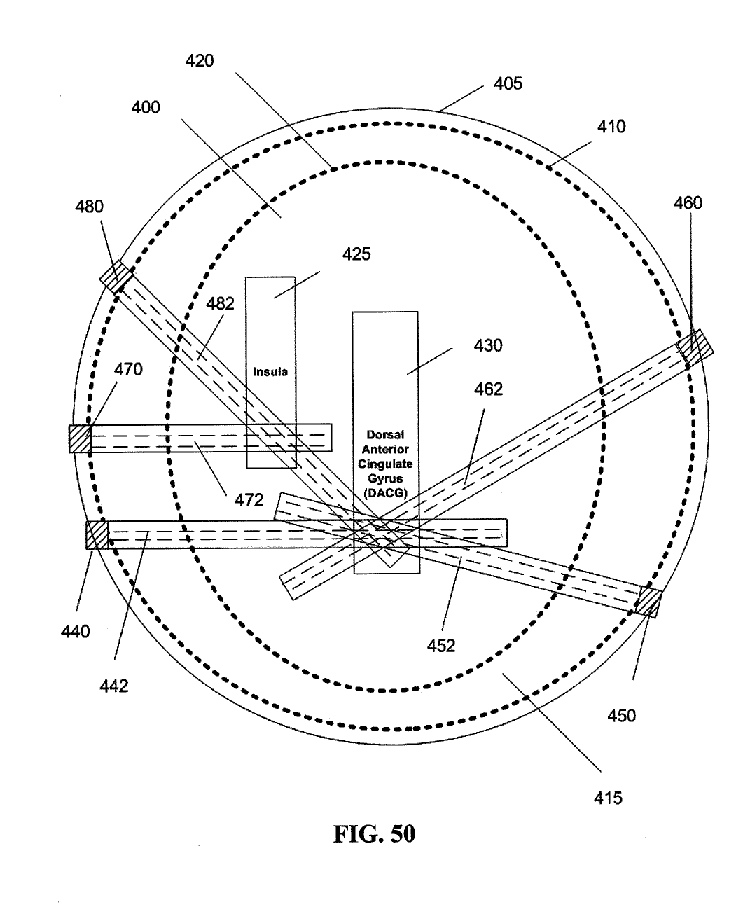

In another embodiment, a plurality of targets is each hit by intersecting ultrasound beams. FIG. 50 illustrates head 400 containing targets Insula 425 and Dorsal Anterior Cingulate Gyms (DACG) 430. Frame 405 holds five ultrasound transducers 440, 450, 460, 470, 480. The beam from each ultrasound transducer passes though a band of ultrasound-conduction medium 415 although in an alternative embodiment the beams can pass through individual ultrasound-conduction media such as shown in FIG. 49. From ultrasound transducer 440, beam 442 passes through ultrasound-conduction medium 415 then into the head, hitting target DACG 430. From ultrasound transducer 450, beam 452 passes through ultrasound-conduction medium 415 then into the head, hitting target DACG 430. In like manner, from ultrasound transducer 460, beam 462 passes through ultrasound-conduction medium 415 then into the head, hitting target DACG 430. Beams 442, 452, and 462 intersect in the Dorsal Anterior Cingulate Gyms 430, enhancing the neuromodulation at that target. Effects of beams 442, 452, and 462 are additive. Ultrasound-conjunction conjunction gel (not shown) can be placed just at the interfaces between any of the ultrasound transducers and the band of ultrasonic-conduction medium 415 and that band and head 400 as long as the beam regions are covered. The other neural target in FIG. 50 is the Insula 425. Targeting the Insula are ultrasound transducers 470 and 480. From ultrasound transducer 470, beam 472 passes through ultrasound-conduction medium 415 then into the head, hitting target Insula 425. From ultrasound transducer 480, beam 482 passes through ultrasound-conduction medium 415 then into the head, hitting target Insula 425. It also will intersect Dorsal Anterior Cingulate Gyms 430 but will have minimal impact because it will be the only ultrasound beam present where it passes through the DACG. Beams 472 and 482 intersect in the Insula 425, enhancing the neuromodulation at that target. Beams 472 and 482 are additive. Beam 482 not only neuromodulates the target Insula 425, but also continues through to neuromodulate DACG 430 where beam 482 intersects beams 442, 452, and 462 from ultrasound transducers 440, 450, and 460. The effects of beams 442, 452, 462, and 482 are additive. The ultrasound transducers can also be used with an acoustic lens (not shown). Again, for elongated targets such as the DACG, the intersecting beams can be spread to cover a broader neural region. In addition the width of the ultrasound transducer and thus the width of the beam can be varied.

In another embodiment, the neuromodulation of one or a plurality of ultrasound transducers is combined with the neuromodulation from one or a plurality of Transcranial Magnetic Stimulation (TMS) electromagnetic coils. In another embodiment, a viewing hole can be placed in an ultrasound transducer to provide an imaging port. Blatek, Imasonic and Keramos-Etalon can supply such configurations.

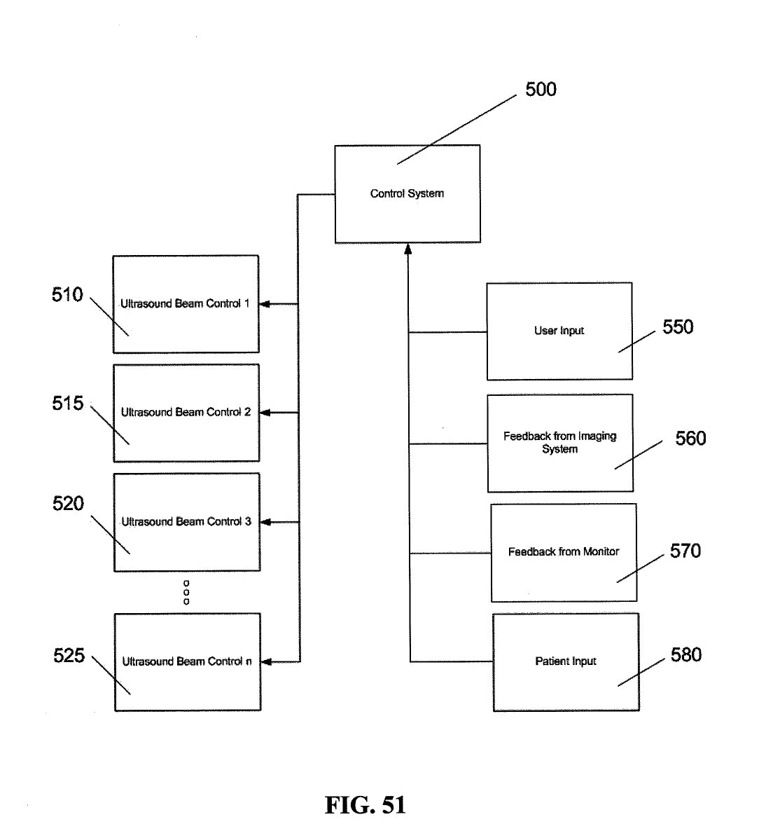

FIG. 51 shows a control block diagram. The direction of the energy emission, intensity, frequency (carrier frequency and/or neuromodulation frequency), pulse duration, pulse pattern, and phase/intensity relationships in targeting for the ultrasonic transducers 510, 515, 520, 525 (and, as applicable, additional ultrasound transducers as indicated by the ellipsis between ultrasound transducers 520 and 525) are controlled by control system 500 with control input from user by user input 550 and/or from feedback from imaging system 560 (either automatically or display to the user with actual control through user input 550), and/or feedback from a monitor (sound and/or thermal) 570, and/or the patient 580 and/or, in the future, other feedback. If positioning of the ultrasound transducers is included as a control element, then control system 550 will control positioning as well.

The invention can be applied to a number of conditions including, but not limited to, addiction, Alzheimer's Disease, anorgasmia, anhedonia, Attention Deficit Hyperactivity Disorder, Autism Spectrum Disorders, Huntington's Chorea, Impulse Control Disorder, OCD, Social Anxiety Disorder, Parkinson's Disease and other motor disorders, Post-Traumatic Stress Disorder, depression, bipolar disorder, pain, insomnia, spinal cord injuries, gastrointestinal motility disorders, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity that can have benefits, for example, in the early treatment of head trauma or other insults to the brain.

All of the embodiments above, except those explicitly restricted in configuration to hit a single target, are capable of and usually would be used for targeting multiple targets either simultaneously or sequentially. Hitting multiple targets in a neural circuit in a treatment session is an important component of fostering a durable effect through Long-Term Potentiation (LTP) and/or Long-Term Depression (LTD) or enhances acute effects (e.g., such as treatment of post-surgical pain). In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in others only one side will targeted (e.g., the right Insula in the case of addiction).

The invention allows stimulation adjustments in variables such as, but not limited to, intensity, firing pattern, and frequency, and position to be adjusted so that if a target is in two neuronal circuits the output of the transducer or transducers can be adjusted to get the desired effect and avoid side effects. Position can be adjusted as well. The side effects could occur because for one indication the given target should be up regulated and for the other down regulated. An example is where a target or a nearby target would be down regulated for one indication such as pain, but up-regulated for another indication such as depression. This scenario applies to either the Dorsal Anterior Cingulate Gyms (DACG) or Caudate Nucleus. Even when a common target is neuromodulated, adjustment of stimulation parameters may moderate or eliminate a problem.

The invention also covers contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyms (DACG). To counterbalance the down regulation of the DACG during treatment for pain that negatively impacts the treatment for depression, one would up regulate the Nucleus Accumbens or Hippocampus that are other targets in the depression neural circuit. A plurality of such applicable targets could be stimulated as well.

Another applicable scenario is the Nucleus Accumbens that is down regulated to treat addiction, but up regulated to treat depression. To counteract the down regulation of the Nucleus Accumbens to treat depression but will negatively impact the treatment of depression that would like the Nucleus Accumbens to be up regulated, one would up regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part X: Ultrasound-Neuromodulation Techniques for Control of Permeability of the Blood-Brain Barrierus

It is the purpose of some of the inventions described herein to provide methods and systems using non-invasive ultrasound-neuromodulation techniques to selectively alter the permeability of the blood-brain barrier (either brain or spinal cord). If the target is a neural target as opposed to a tumor, the application of the invention may result in effective neuromodulation of that target in addition to altering the permeability of the blood-brain barrier in that region allowing more effective penetration of a drug to impact that neural target. This applies to humans or animals and in brain or spinal cord. The change can control blood-brain permeability by increasing permeability to increase the access of drugs to, for example, neurological targets or tumors or decreasing permeability to protect targets from drugs that could cause side effects. If the application of the techniques results in decreasing the permeability of the blood-brain barrier (in cases where the permeability has been increased through another mechanism), in some cases coincident neuromodulation of a target in the region will have a therapeutic benefit. Multiple conditions are aggravated by breaching of the blood-brain barrier, among which are Alzheimer's Disease, HIV Encephalitis, Multiple Sclerosis, Meningitis, and Epilepsy. Such neuromodulation systems can produce applicable acute or long-term effects. The latter occur through Long-Term Depression (LTD) or Long-Term Potentiation (LTP) via training. Included is control of direction of the energy emission, intensity, frequency (carrier and/or neuromodulation frequency), pulse duration, firing pattern, and phase/intensity relationships for beam steering and focusing on targets and accomplishing up-regulation and/or down-regulation.

What will work for altering the permeability of the blood brain barrier in a given situation depends on a given patient and associated condition. In some situations, excitation will result in increasing the permeability of the blood-brain barrier and inhibition will result in decreasing it. In other situations, the reverse will be true.

Ultrasound is acoustic energy with a frequency above the normal range of human hearing (typically greater than 20 kHz). In this invention, ultrasound-neuromodulation techniques refers to the delivery of ultrasound energy to tissue in the brain or spinal cord having an acoustic frequency in a range of 0.3 MHz to 0.8 MHz with acoustic intensity greater than 20 mW/cm² at the target tissue. The frequency in the range of 0.3 MHz to 0.8 MHz represents the carrier frequency on which amplitude modulation is applied. The amplitude modulation frequency for inhibition or down regulation is typically lower than 500 Hz (depending on condition and patient). The amplitude modulation frequency for excitation is typically in the range of 500 Hz to 5 MHz again depending on condition and patient. In one embodiment, the modulation frequency of lower than approximately 500 Hz is divided into pulses 0.1 to 20 msec. repeated at frequencies of 2 Hz or lower for inhibition or down regulation. In one embodiment, the amplitude modulation frequency of higher than approximately 500 Hz is divided into pulses 0.1 to 20 msec. repeated at frequencies higher than 2 Hz for up regulation. In some embodiments the acoustic intensity is greater than about 30 mW/cm² at the target tissue. The acoustic intensity is less than the appropriate target- or patient-specific levels at which no tissue damage is caused. Ultrasound therapy can be combined with therapy using other devices Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space between the ultrasound transducer and the head of a subject.

Altering the permeability of the blood-brain barrier using ultrasound-neuromodulation techniques has significant benefits over other techniques such as Transcranial Magnetic Stimulation neuromodulation (e.g., using the Brainsway system) because ultrasound neuromodulation provides greater resolution and uses hardware that is both less expensive and portable so it can be used at home or other non-clinical-office locations.

A notable benefit is the ability to reduce side effects by having increased permeability in applicable regions where a drug needs to be active and leave at its normal level or decrease permeability in other regions where that drug could cause side effects. This spatial selectivity depends on the ability of the neuromodulation to be selective which is true for ultrasound neuromodulation, but not true for an essentially whole-brain neuromodulation approach such as that of Brainsway or any approach using Transcranial Magnetic Stimulation. Another facet of side effects is the significant opportunity to protect structures by selectively decreasing the permeability in certain regions.

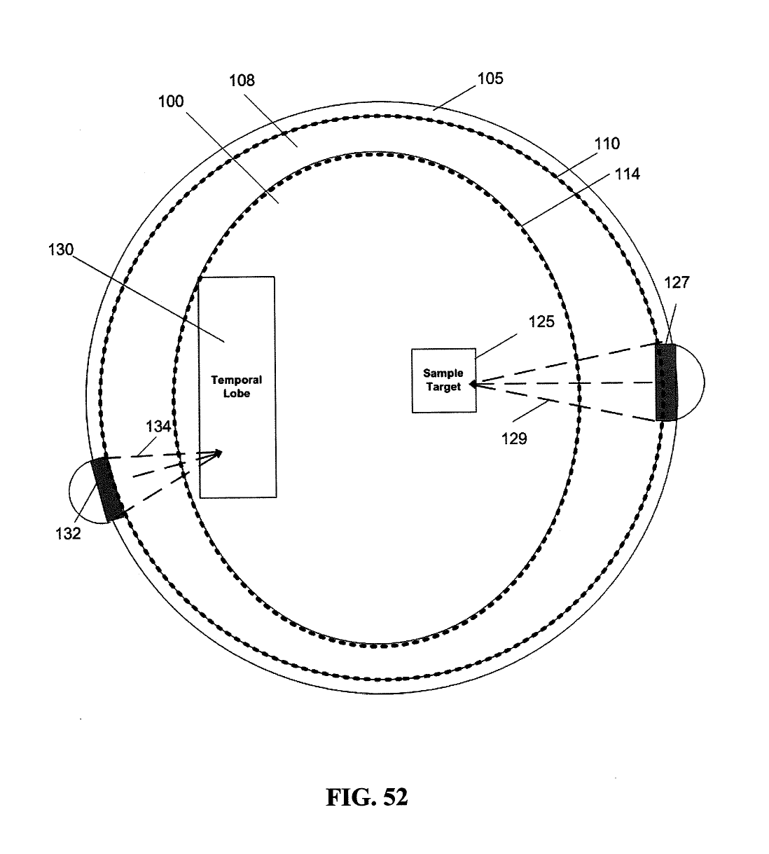

FIG. 52 shows exemplar targets for control of permeability of the blood-brain barrier for the selective penetration of drugs or other substances into the target. Head 100 contains two targets, one a generic Sample Target 125 and the other the Temporal Lobe 130 as an example of a neural target for the treatment of epilepsy. For example, Sample Target 125 may represent a malignant tumor such as glioblastoma multiforme (the subject of the work by Brainsway) to open up the path for anti-tumor drugs and Temporal Lobe 130 would be a target for permeability change to open up the path for anti-epilepsy drugs. There can be different numbers of targets for a given condition and the appropriate targets will change as research evolves. Targets 125 and 130 are targeted by ultrasound from transducers 127 and 132 respectively, fixed to track 105. In other embodiments the ultrasound transducer or transducers can be affixed to the patient's head using other means such as strapping to the head or holding within the framework of a swimming-cap-style structure. Ultrasound transducer 127 with its beam 129 is shown targeting Sample Target 120 and transducer 132 with its beam 134 is shown targeting Temporal Lobe 130. Bilateral stimulation of one of a plurality of these targets is another embodiment. For ultrasound to be effectively transmitted to and through the skull and to brain targets, coupling must be put into place. Ultrasound transmission (for example Dermasol from California Medical Innovations) medium 108 is interposed with one mechanical interface to the frame 105 and ultrasound transducers 127 and 132 (completed by a layer of ultrasound transmission gel layer 110) and the other mechanical interface to the head 100 (completed by a layer of ultrasound transmission gel 114). In another embodiment, the ultrasound transmission gel is only placed at the particular places where the ultrasonic beams from the transducers are located rather than around the entire frame and entire head. In another embodiment, multiple ultrasound transducers whose beams intersect at that target replace an individual ultrasound transducer for that target. If a large volume of the brain is to have its permeability altered then multiple ultrasound transducers with defocused beams can be employed.

Transducer array assemblies of this type may be supplied to custom specifications by Imasonic in France (e.g., large 2D High Intensity Focused Ultrasound (HIFU) hemispheric array transducer) (Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of ultrasound transducers of 300 or more. Keramos-Etalon and Blatek in the U.S. are other custom-transducer suppliers. The power applied will determine whether the ultrasound is high intensity or low intensity (or medium intensity) and because the ultrasound transducers are custom, any mechanical or electrical. changes can be made, if and as required. At least one configuration available from Imasonic (the HIFU linear phased array transducer) has a center hole for the positioning of an imaging probe. Keramos-Etalon also supplies such configurations.

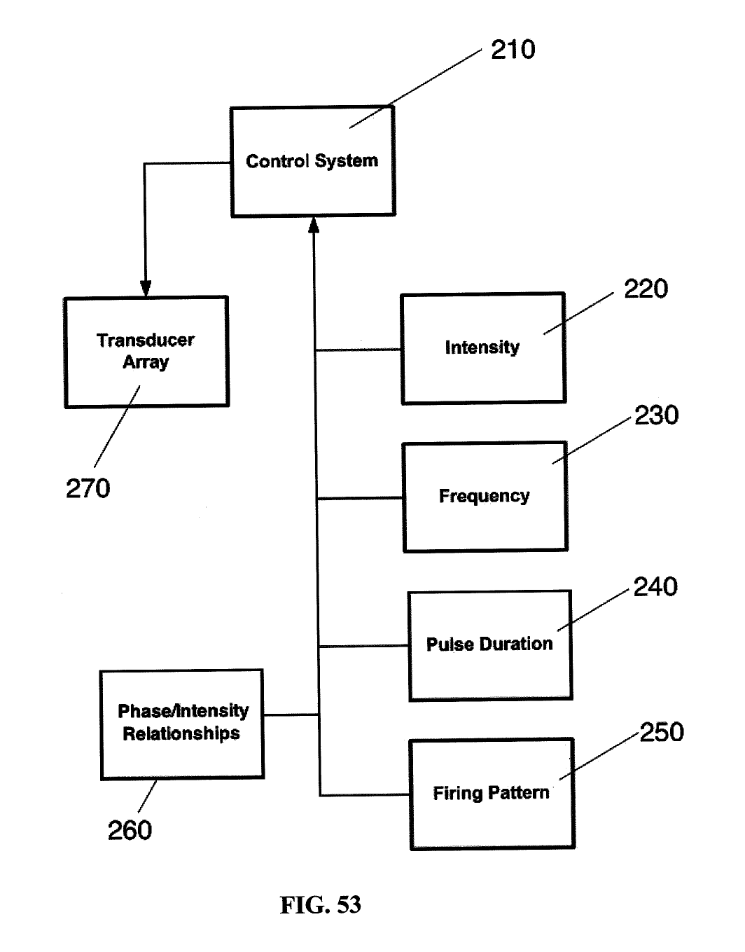

FIG. 53 shows an embodiment of a control circuit. The positioning and emission characteristics of transducer array 270 are controlled by control system 210 with control input with neuromodulation characteristics determined by settings of intensity 220, frequency 230 (can be carrier and/or neuromodulation frequency), pulse duration 240, firing pattern 250, and phase/intensity relationships 460 for beam steering and focusing on neural targets. Instead of phase/frequency relationships that can steer the ultrasound beam, 260 can represent mechanically altering the direction of the ultrasound beam, including axial or radial mechanical perturbations of the ultrasound transducers.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

The invention allows stimulation adjustments in variables such as, but not limited to, intensity, firing pattern, frequency (carrier and/or neuromodulation; frequency), pulse duration, firing pattern, phase/intensity relationships for beam steering, dynamic sweeps, position, and direction, including axial or radial perturbations of the ultrasound transducers.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part XI: Ultrasound Neuromodulation of Spinal Cord

It is the purpose of some of the inventions described herein to provide methods and systems and methods for neuromodulation of the spinal cord to treat certain types of pain. Such pain conditions include non-cancer pain, failed-back-surgery syndrome, reflex sympathetic dysthropy (complex regional pain syndrome), causalgia, arachnoiditis, phantom limb/stump pain, post-laminectomy syndrome, cervical neuritis pain, neurogenic thoracic outlet syndrome, postherpetic neuralgia, functional bowel disorder pain (including that found in irritable bowel syndrome), and refractory pain due to ischemia (e.g. angina). In certain embodiments of the present invention, pain is replaced by tingling parathesias. In certain embodiments of the present invention, ultrasound neuromodulation stimulates pain inhibition pathways and can produce acute or long-term effects. The latter occur through long-term depression (LTD) or long-term potentiation (LTP) via training. Acute and chronic vasculitis can be treated as well as associated pain. In addition, sacral neuromodulation can be employed for the treatment of hyperactive bladder as well as to stimulate emptying of a neurogenic bladder. Included is control of direction of the energy emission, intensity, frequency (carrier frequency and/or neuromodulation frequency), pulse duration, pulse pattern, and phase/intensity relationships to targeting and accomplishing up-regulation and/or down-regulation.

Target regions in the spinal cord which can be treated using the ultrasound neuromodulation protocols of the present invention comprise the same locations targeted by electrical SCS electrodes for the same conditions being treated, e.g., a lower cervical-upper thoracic target region for angina, a T5-7 target region for abdominal/visceral pain, and a T10 target region for sciatic pain. Ultrasound neuromodulation in accordance with the present invention can stimulate pain inhibition pathways which in turn can produce acute and/or long-term effects. Other clinical applications of ultrasound neuromodulation of the spinal cord include non-invasive assessment of neuromoduation at a particular target region in a patient's spinal cord prior to implanting an electrode for electrical spinal cord stimulation for pain or other conditions.

The stimulation frequency for inhibition may be lower than 500 Hz (depending on condition and patient). The stimulation frequency for excitation may be above 500 Hz, typically being in the range of 500 Hz to 5 MHz. In this invention, the ultrasound acoustic frequency is in range of 0.3 MHz to 0.8 MHz with power generally applied less than 60 mW/cm2 usually less than 21 mW/cm2, often less than 10 mW/cm2. The acoustic frequency is modulated at the lower rate to impact the neuronal structures as desired (e.g., 300 Hz for inhibition (down-regulation) or 1 kHz for excitation (up-regulation). The modulation frequency (superimposed on the carrier frequency of say 0.5 MHz or similar) may be divided into pulses 0.1 to 20 msec repeated at frequencies of 2 Hz or lower for down regulation and higher than 2 Hz for up regulation) although this will be both patient and condition specific. The number of ultrasound transducers can vary between one and 500.

The lower size boundary of the spot or line width of the focused ultrasound energy will depend on the ultrasonic frequency, with higher frequencies generally corresponding to smaller spots or widths. Ultrasound-based neuromodulation operates preferentially at low frequencies relative to say imaging applications so there is less resolution. A suitable one-inch diameter ultrasound transducer having a focal length of two inches that operates with a 0.4 Mhz excitation frequency and will deliver a focused spot with a diameter (6 dB) of 0.29 inches is available from Keramos-Etalon. Typically, the spot size will be in the range of 0.1 inch to 0.6 inch depending on the specific indication and patient. A larger spot can be obtained with a one-inch diameter ultrasound transducer with a focal length of 3. inch which operates at 0.4 MHz excitationand will deliver a focused spot with a diameter (6 dB) of 0.51 inch. Even though the target is relatively superficial, the transducer can be moved back in the holder to allow a longer focal length. Other embodiments are applicable as well, including different transducer diameters, different frequencies, and different focal lengths. Other ultrasound transducer manufacturers include Blatek and Imasonic. In an alternative embodiment, focus can be deemphasized or eliminated with a smaller ultrasound transducer diameter with a shorter longitudinal dimension, if desired, as well. Ultrasound conduction medium will usually be provided to fill the space between the transducer and the patient's skin.

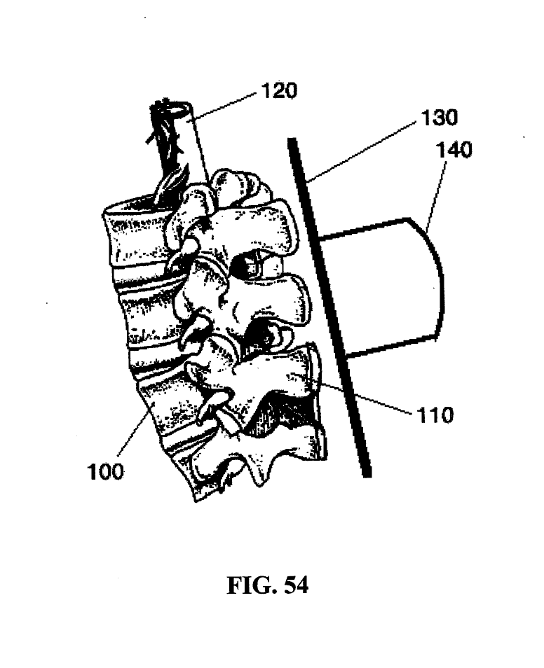

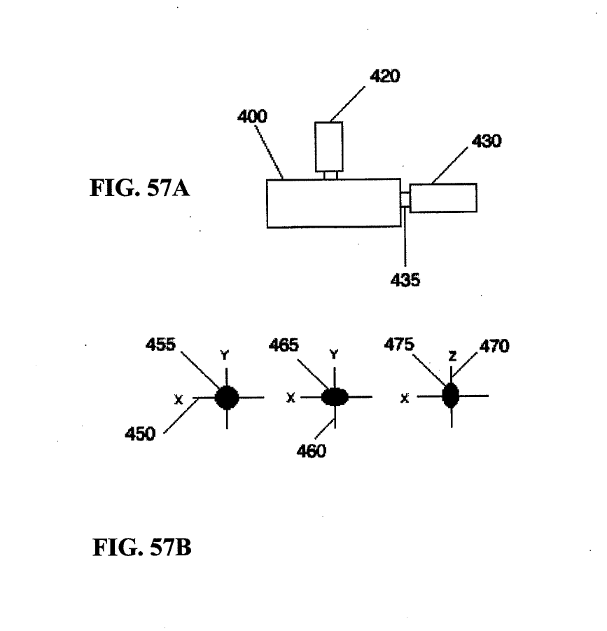

FIG. 54 shows spinal column with vertebrae 100 and spinal process 110 containing spinal cord 120 covered by skin 130. Spinal cord 120 is neuromodulated by ultrasound transducer 140. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing or between the transducer face and the skin), an ultrasound transmission medium (for example Dermasol from California Medical Innovations) can be used. In another embodiment, multiple ultrasound transducers whose beams intersect at that target replace an individual ultrasound transducer for that target. Transducers can be placed on both sides of the spinous processes to direct beams inwardly to integrate along the spinal cord or can be located on one side only and focused medially to target the spinal cord. In still another embodiment, mechanical perturbations are applied radially or axially to move the ultrasound transducers, as discussed below with reference to FIGS. 57A and 57B.

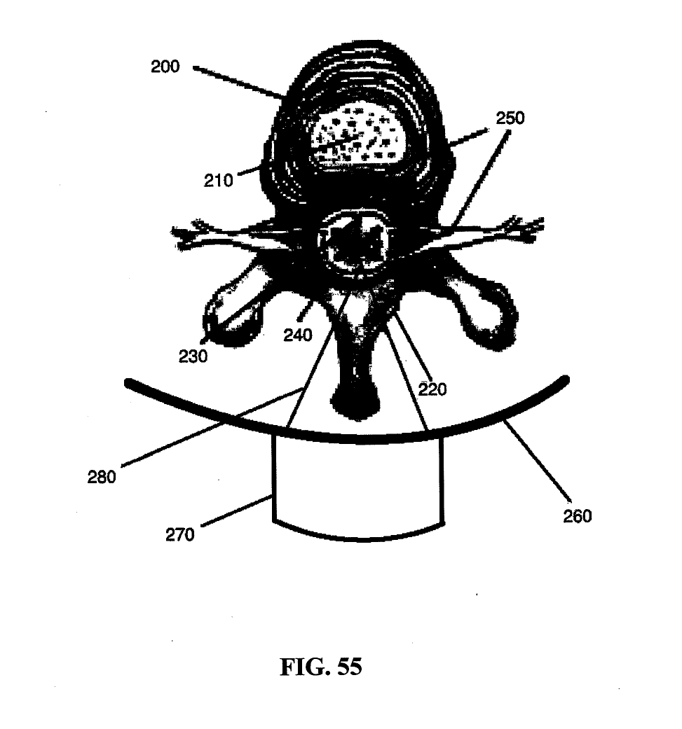

FIG. 55 shows a cross section of the spinal column and spinal cord. Vertebrae disc 200 with its nucleus pulposus 210 with other bony structures such as the lamina 220 surrounds the dura 240 surrounding spinal cord 230 with its spinal nerve roots 250. Ultrasound transducer 270 is pressed against skin 260 and generates ultrasound beam 280 that neuromodulates nerves within spinal cord 230. Bilateral neuromodulation of spinal cord 230 can be performed. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing), an ultrasound transmission medium (for example Dermasol from California Medical Innovations) can be used. In another embodiment, multiple ultrasound transducers whose beams intersect at that target replace an individual ultrasound transducer for that target. In still another embodiment, mechanical perturbations are applied radially or axially to move the ultrasound transducers (FIGS. 57A-57B).

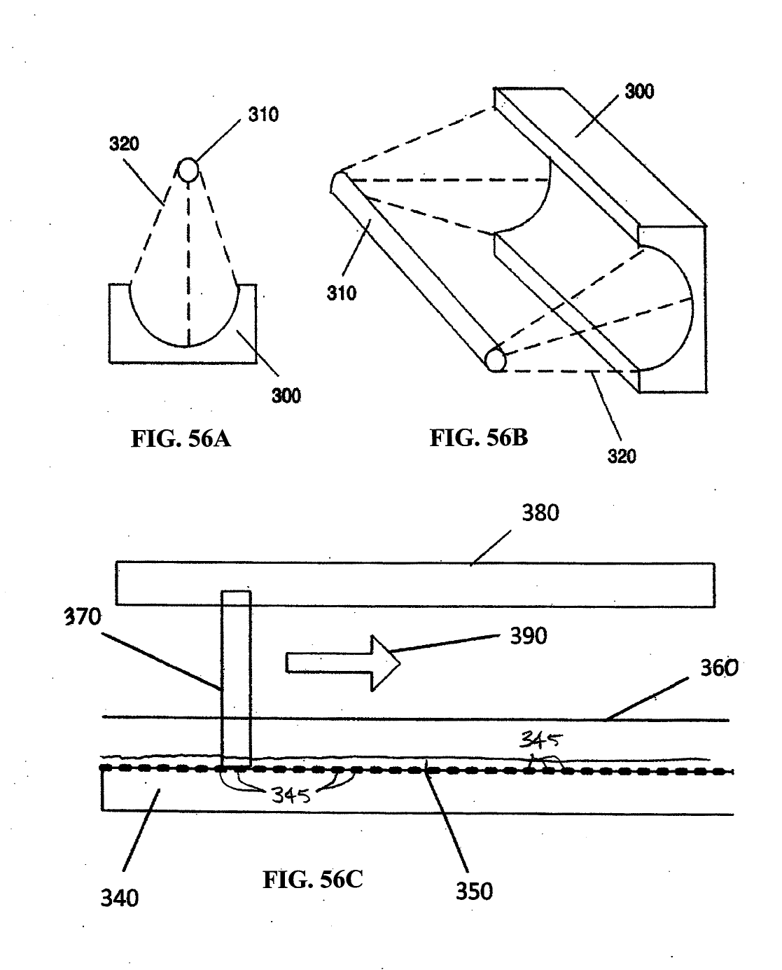

FIGS. 56A and 56B show an exemplary ultrasound transducer assembly 300 that may be a shaped piezoelectric transducer body or may comprise an array of individual transducer elements configured to produce an elongated tubular (e.g. pencil-shaped) focused field 310. Such a transducer assembly is applied to stimulate an elongated target such as the spinal cord. In alternative embodiments, a spot focused ultrasonic energy beam may be over any portion of the length of the spinal cord to target specific target regions. In both cases, it is possible to determine over what length of a target region that the ultrasound is to be applied. For example, one could apply ultrasound to only a selected portion of the spinal cord. In FIG. 56A, an end view of the array is shown with curved-cross section ultrasonic array 300 forming a sound field 320 focused on target 310. FIG. 56B shows the same array in a side view, again with ultrasound array 300, target 310, and focused field 320.

FIG. 56C shows a linear ultrasound phased array 340 which can “steer” an ultrasound beam 370 by changing the phase/intensity relationships of a plurality of individual transducer elements 345. In this way, ultrasound beams can be moved (steered) and focused without physically displacing the array 340 of transducers 345. The beam direction can be directed at angles which are perpendicular or non-perpendicular to the surface of the transducer array, and beam direction is thus not restricted to being aimed perpendicularly from the face of the transducer or array. In FIG. 56C, the transducer array 340 is flat and emits ultrasound conducted by a conducting gel layer 350 providing the physical interface to skin over spinal column 360. The beam 370 of ultrasound energy moves linearly from left to right as shown by arrow 390 so it moves its focus along spinal cord target 380. Transducers can be place on either side of the spinous processes or placed on one side and aimed medially. In still another embodiment, mechanical perturbations may be applied to move the ultrasound transducers as covered in FIGS. 57A-57B, for example, to increase ultrasound field depth. In another embodiment, the surface of the transducer array is not flat but curved.

Transducer array assemblies of this type may be supplied with custom specifications by Imasonic in France (e.g., large 2D High Intensity Focused Ultrasound (HIFU) hemispheric array transducer; and Fleury G., Berriet, R., Le Baron, O., and B. Huguenin, “New piezocomposite transducers for therapeutic ultrasound,” 2nd International Symposium on Therapeutic Ultrasound—Seattle—31/07—Feb. 8, 2002), typically with numbers of ultrasound transducers of 300 or more. Keramos-Etalon in the United States is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

FIGS. 57A and 57B show the mechanism for mechanical perturbation of the ultrasound transducer. In FIG. 57A illustrating a plan view with mechanical actuators 420 and 430 moving ultrasound transducer 400 in and out and left respectively. Actuator rod 435 provides the mechanical interface between mechanical actuator 430 and ultrasound transducer 400 as an example. Not shown is an equivalent mechanical actuator moving ultrasound transducer 400 along an axis perpendicular to the page. Such mechanical actuators can have alternative configurations such as motors, vibrators, solenoids, magnetostrictive, electrorestrictive ceramic and shape memory alloys. Piezo-actuators such as those provided by DSM can have very fine motions of 0.1% length change. FIG. 57B shows effects on the focused ultrasound modulation focused at the target. The axes are 450 (x,y), 460 (x,y,) and 470 (x,z). As demonstrated on 450 the excursions along x and y from 430 and 420 are equal so the resultant pattern is a circle. As demonstrated on 460 the excursion due to 430 is greater than that if 420 so the resultant pattern is longer along the x axis than the y axis. As demonstrated on 470, the excursion is longer along the z axis than the x axis to the resultant pattern is long along the z axis than the x axis. Not shown is the inclusion of the impacts of actuation along the axis perpendicular to the page. In each case, the pattern would be matched to the shape of the target of the modulation. For the transducer arrangement shown in FIGS. 56A and 56B, depth can be added to the length and width which are produced.

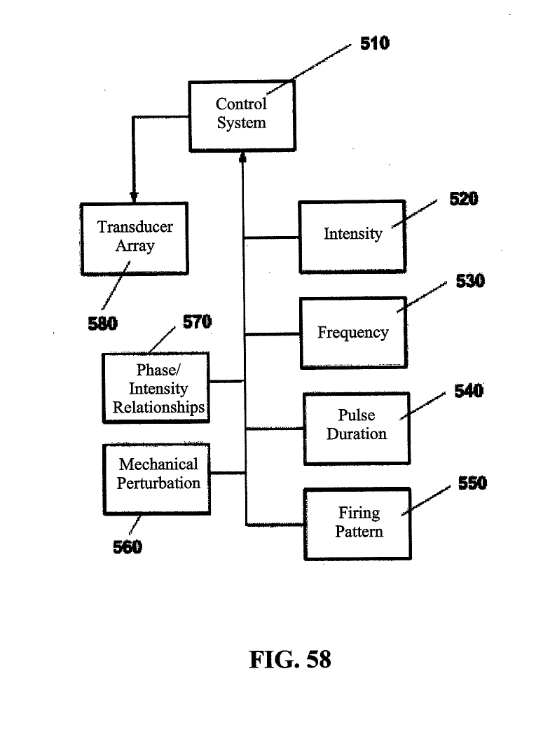

FIG. 58 shows an embodiment of a control circuit. The positioning and emission characteristics of transducer array 580 are controlled by control system 510 with control input with neuromodulation characteristics determined by settings of intensity 520, frequency (including carrier frequency) 530, pulse duration 540, firing pattern 550, mechanical perturbation 560, and phase/intensity relationships 570 for beam steering and focusing on neural targets.

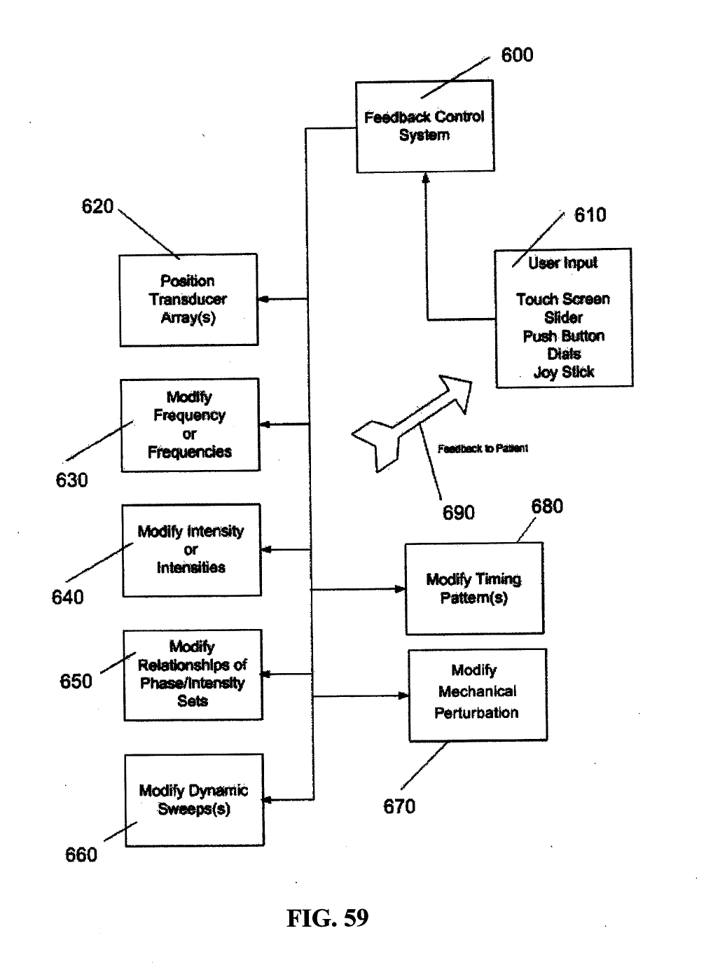

The operator can set the variables for the ultrasound neuromodulation or the patient can do so. FIG. 59 shows the basic feedback circuit. Feedback Control System 600 receives its input from User Input 610 and provides control output for positioning ultrasound transducer arrays 620, modifying pulse frequency or frequencies 630, modifying intensity or intensities 640, modifying relationships of phase/intensity sets 650 for focusing including spot positioning via beam steering, modifying dynamic sweep patterns 660, modifying mechanical perturbation 670, and/or modifying timing patterns 680. Feedback to the patient 690 occurs with what is the physiological effect on the patient (for example increase or decrease in pain or decrease or increase on tremor). User Input 610 can be provided via a touch screen, slider, dials, joystick, or other suitable means. Often the user can be the best judge what alterations of what changes in neuromodulation will be most helpful, either changing one variable at a time or multiple variables. One example of patient control is the patient (e.g., one with a transected spinal cord) directly turning on the neuromodulation to empty a neurogenic bladder.

In still other embodiments, other energy sources are used in combination with or substituted for ultrasound transducers that are selected from the group consisting of Transcranial Magnetic Stimulation (TMS), Spinal Cord Stimulation (SCS), and medications.

The invention allows stimulation adjustments in variables such as, but not limited to, direction of the energy emission, intensity, frequency (carrier frequency and/or neuromodulation frequency), pulse duration, pulse pattern, and phase/intensity relationships to targeting and accomplishing up-regulation and/or down-regulation, dynamic sweeps, mechanical perturbation, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

### Part XII: Ultrasound Neuromodulation for Diagnosis and Other-Modality Preplanning

The embodiments as described herein provide methods and systems for non-invasive neuromodulation using ultrasound to one or more of diagnosis or to evaluate the feasibility of and preplan neuromodulation treatment using other modalities, such as drugs, electrical stimulation, transcranial ultrasound neuromodulation, surgical intervention, transcranial direct current stimulation, optogenetics, implantable devices, or implantable electrodes and combinations thereof, for example.

In many embodiments, the patient can be diagnosed by selecting one or more target sites. The one or more sites are provided with the focused ultrasound beam. An evaluation of the elicited response to the ultrasound beam may be used to distinguish between one or more patient disorders. The patient treatment can be guided by the disorder identified. The guided treatment may comprise one or more of drugs, neuromodulation, or surgery, for example.

In many embodiments confirming a treatment site encompasses determining which of one or more target neural sites can effectively treat the symptoms to be mitigated, based on identification of the one or more target sites from among a plurality of possible target sites based on a response of the patient to the focused ultrasound beam applied to one or more of the possible target sites.

In many embodiments, the confirmed target site is treated with the non-ultrasonic treatment modality after the confirmed target has been determined to be effective based on the patient's response to focused ultrasonic beam delivered to the target site. In many embodiments, the confirmed target site comprises a target site determined to be most likely to successfully treat the patient. The confirmed target site can be selected from among a plurality of possible target sites evaluated based on the response of the patient to the focused ultrasonic beam.

In many embodiments, the confirmation that treatment at a specific site is effective based on ultrasound occurs before implanting the electrode or other implantable device, for example.

The confirmation of the target site allows one to determine which neural target or targets among a plurality of potential targets will most effectively deal with the symptoms to be mitigated. Such neuromodulation systems can produce applicable acute or long-term effects. The long-term effects can occur through Long-Term Depression (LTD) or Long-Term Potentiation (LTP) via training, for example. The embodiments described herein provide control of direction of the energy emission, intensity, frequency (carrier frequency and/or neuromodulation frequency), pulse duration, pulse pattern, and phase/intensity relationships to targeting and accomplishing up-regulation and/or down-regulation, for example.

In some embodiments, the stimulation frequency for inhibition may be lower than 500 Hz (depending on condition and patient). In an embodiment of the invention, the stimulation frequency for excitation is in the range of 500 Hz to 5 MHz. In an embodiment, the ultrasound acoustic carrier frequency is in range of 0.3 MHz to 0.8 MHz with power generally applied less than 60 mW/cm2 but also at higher target- or patient-specific levels at which no tissue damage is caused. In other embodiments, the ultrasound acoustic carrier frequency can be in range of 0.1 MHz to 0.3 MHz. Alternatively or in combination, the ultrasound acoustic carrier frequency can be in range of 0.8 MHz to 10 MHz, for example. The stimulation frequency can be provided by modulating the ultrasound acoustic carrier frequency with the stimulation frequency, for example.

In many embodiments, the lower limit of the spatial-peak temporal-average intensity (Ispta) of the ultrasound energy at a target tissue site is chosen from the group of: 21 mW/cm2, 25 mW/cm2, 30 mW/cm2, 40 mW/cm2, or 50 mW/cm2, for example. In an embodiment of the invention, the upper limit of the Ispta of the ultrasound energy at a target tissue site is chosen from the group of: 1000 mW/cm2, 500 mW/cm2, 300 mW/cm2, 200 mW/cm2, 100 mW/cm2, 75 mW/cm2, or 50 mW/cm2.

In an embodiment of the invention, the acoustic frequency is modulated so as to impact the neuronal structures as desired (e.g., say typically 300 Hz for inhibition (down-regulation) or 1 kHz for excitation (up-regulation), for example).

In many embodiments, the modulation frequency may be divided into pulses 0.1 to 20 msec, and the modulation frequency may be superimposed on the ultrasound carrier frequency, which can be about 0.5 MHz, for example.

In an embodiment, the pulses are repeated at frequencies of 2 Hz or lower for down regulation and higher than 2 Hz for up regulation although this will be both patient and condition specific.

The number of ultrasound transducers can vary between one and five hundred, for example.

In many embodiments, ultrasound therapy is combined with therapy using other neuromodulation modalities, such as one or more of Transcranial Magnetic Stimulation (TMS) or transcranial Direct Current Stimulation (tDCS), for example.

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a known commercially available 1-inch diameter ultrasound transducer and a focal length of 2 inches that will deliver a focused spot with a diameter (6 dB) of 0.29 inches with 0.4 MHz excitation. In many embodiments, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

The ultrasound neuromodulation can be administered in sessions. Examples of session types include periodic sessions, such as a single session of length in the range from 15 to 60 minutes repeated daily or five days per week for one to six weeks. Other lengths of session or number of weeks of neuromodulation are applicable, such as session lengths from 1 minute up to 2.5 hours and number of weeks ranging from one to eight. Sessions occurring in a compressed time period typically means a single session of length in the range from 30 to 60 minutes repeated during with inter-session times of 15 minutes to 60 minutes over one to three days. Other inter-session times in the range between 1 minute and three hours and days of compressed therapy such as one to five days are applicable. In an embodiment of the invention, sessions occur only during waking hours. Maintenance consists of periodic sessions at fixed intervals or on as-needed basis such as occurs periodically for tune-ups. Maintenance categories are maintenance post-completion of original treatment at fixed intervals and maintenance post-completion of original treatment with as-needed maintenance tune-ups as defined by a clinically relevant measurement. In an embodiment that uses fixed intervals to determine when additional ultrasound neuromodulation sessions are delivered, one or more 50-minute sessions occur during the second week the 4th and 8th months following the first treatment. In an embodiment that when additional ultrasound neuromodulation sessions are delivered based on a clinically-relevant measurement, one or more 50-minute sessions occur during week 7 because a tune up is needed at that time as indicated by the re-emergence of symptoms. Use of sessions is important for the retraining of neural pathways for change of function, maintenance of function, or restoration of function. Retraining over time, with intermittent reinforcement, can more effectively achieve desired impacts. Efficient schedules for sessions are advantageous so that patients can minimize the amount of time required for their ultrasound treatments. Such neuromodulation systems can produce applicable acute or long-term effects. The latter occur through Long-Term Depression (LTD) or Long-Term Potentiation (LTP) via training.

Work in relation to embodiments as described herein suggests that differences in FUP phase, frequency, and amplitude produce different neural effects. Low frequencies (defined as below 500 Hz.) can be inhibitory in at least some embodiments. High frequencies (defined as being in the range of 500 Hz to 5 MHz) can be excitatory and activate neural circuits in at least some embodiments. In many embodiments, this targeted inhibition or excitation based on frequency works for the targeted region comprising one or more of gray or white matter. Repeated sessions may result in long-term effects. The cap and transducers to be employed can be preferably made of non-ferrous material to reduce image distortion in fMRI imaging, for example. In many embodiments, if after treatment the reactivity as judged with fMRI of the patient with a given condition becomes more like that of a normal patient, this clinical assessment may be indicative of treatment effectiveness. In many embodiments, the FUP is to be applied 1 ms to 1 s before or after the imaging. Alternatively or in combination, a CT (Computed Tomography) scan can be run to gauge the bone density and structure of the skull, which can be used to determine one or more of the carrier wave frequency, the pulse intensity, the pulse energy, the pulse duration, the pulse repetition rate, or the pulse phase, for a series of pulses as described herein, for example.

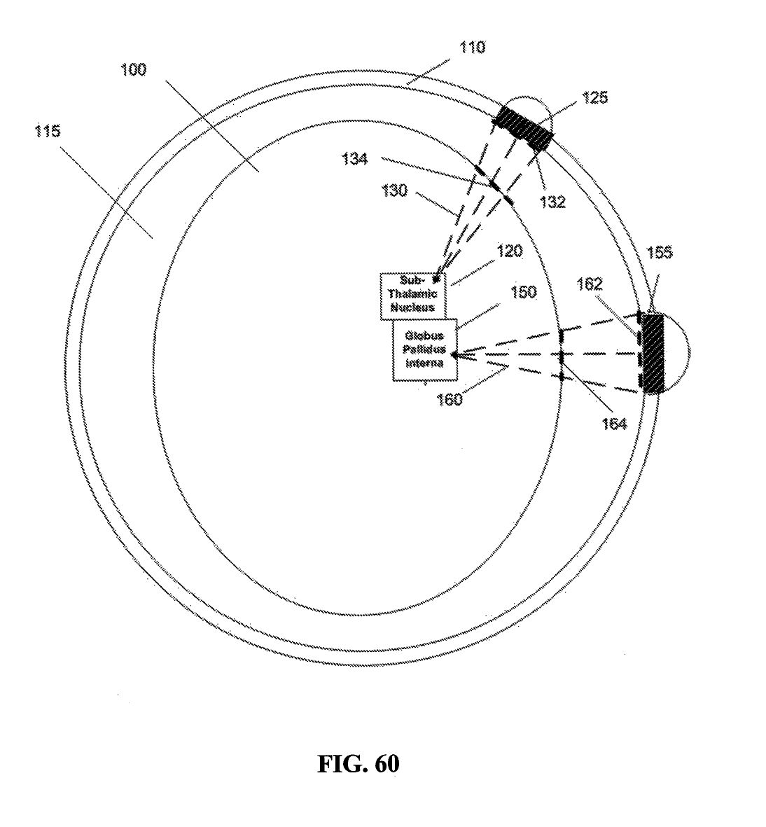

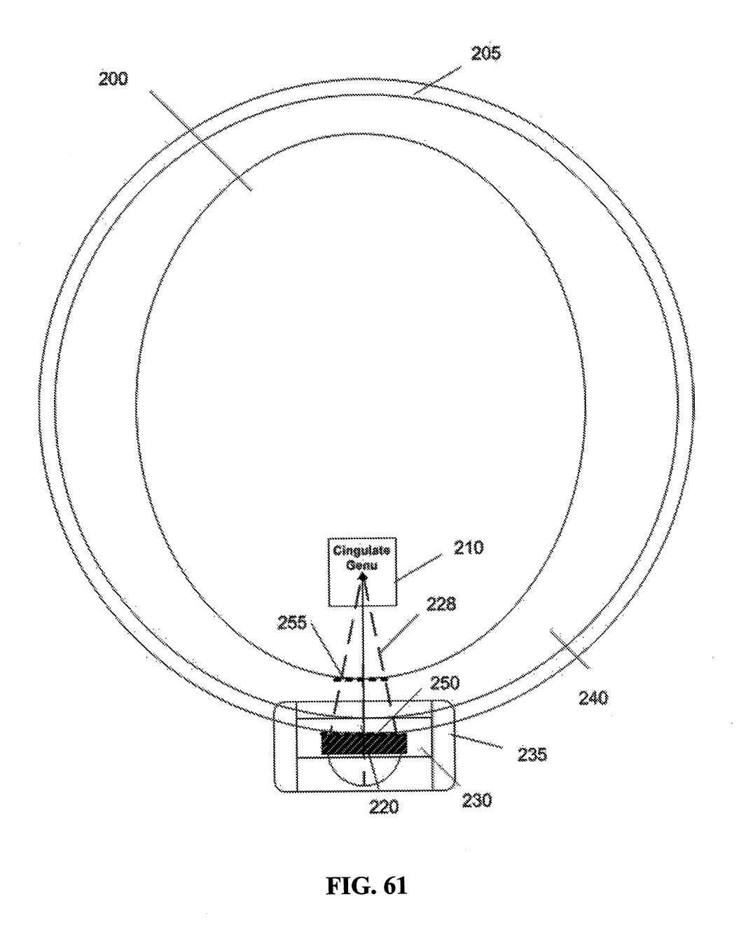

FIG. 60 shows a set of ultrasound transducers targeted to treat Parkinson's Disease. Head 100 contains two targets, Subthalamic Nucleus 120 and Globus Pallidus internal 150. The targets shown are hit by ultrasound from transducers 125 and 155 fixed to track 110. Ultrasound transducer 125 with its beam 130 is shown targeting Subthalamic Nucleus (STN) 120 and transducer 155 with its beam 160 is shown targeting Globus Pallidus internal 150. For ultrasound to be effectively transmitted to and through the skull and to brain targets, coupling must be put into place. Ultrasound transmission (for example Dermasol from California Medical Innovations) medium 115 is interposed with one mechanical interface to the frame 110 and ultrasound transducers 125 and 155 (completed by a layers of ultrasound transmission gel 132 and 162 respectively) and the other mechanical interface to the head 100 (completed by a layers of ultrasound transmission gel 134 and 164 respectively). In another embodiment the ultrasound transmission gel is placed around the entire frame and entire head. In another embodiment, multiple ultrasound transducers whose beams intersect at that target replace an individual ultrasound transducer for that target. In still another embodiment, mechanical perturbations are applied radially or axially to move the ultrasound transducers. In still another embodiment, an alternative target can be evaluated with ultrasound neuromodulation, such the Vim (Ventral Intermediate Nucleus of the Thalamus). A diagnostic application of the invention is the differentiation between the tremor of Parkinson's Disease and essential tremor. Note that one strategy is to use DBS on both the STN and the Vim on the same side. In another embodiment, ultrasound neuromodulation of the spinal cord is used to evaluate the potential effectiveness of or parameters for Spinal Cord Stimulation (SCS) using invasive electrode stimulation for the relief of pain.

FIG. 61 illustrates the Cingulate Genu as a target for testing in a neuromodulation patient to evaluate whether neuromodulation of that target is effective for the mitigation of depression or bipolar disorder. Head 200 is surrounded by head frame 205 on which ultrasound neuromodulation transducer frame 235 containing an adjustment support 230 which moves radially in and out of transducer frame 235. Support 230 holds ultrasound transducer 220 with its ultrasound beam 228 hitting target being evaluated Cingulate Genu 210. In order for the ultrasound beam 228 to penetrate effectively, an ultrasound conduction path must be used. This path consists of ultrasound conduction medium 240 (for example Dermasol from California Medical Innovations) bounded by ultrasound conduction-gel layer 250 on the ultrasound-transducer side and layer 255 on the head side. If the ultrasound neuromodulation is successful, then an alternative neuromodulation modality (e.g., DBS) likely can be used successfully due to smaller targeting area achieved. If the ultrasound neuromodulation of this target is not effective then it is likely that the alternative modality being considered (e.g., DBS) will not be successful with this target. Thus the probability of success with an alternative (potentially invasive) neurmodulation modality can be evaluated. If an acute session of ultrasound neuromodulation is ineffective for alleviating symptoms, then the probability is lower that the patient will benefit from a more invasive procedure such as invasive DBS, avoiding both risk for side effects in the patient and significant cost.

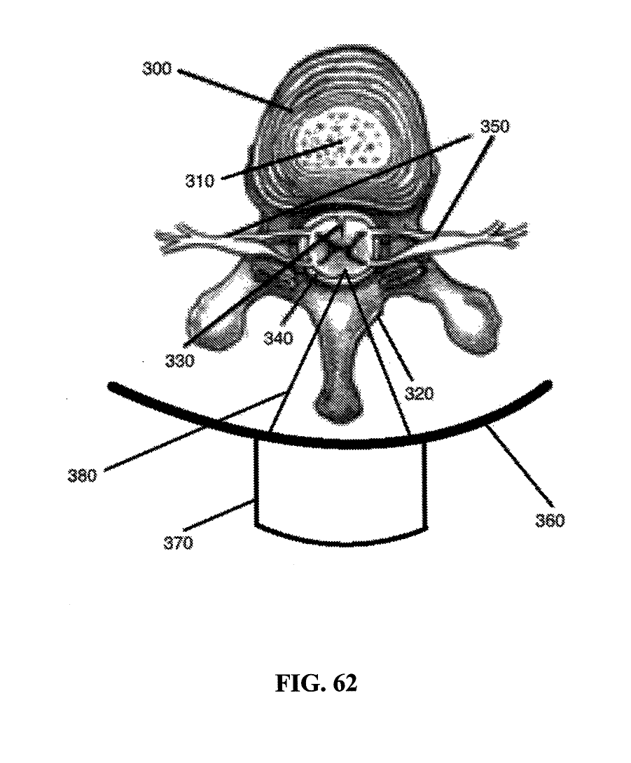

FIG. 62 shows a cross section of the spinal column and spinal cord. Applying ultrasound neuromodulation in this configuration is useful for preplanning to evaluate whether electrode-based Spinal Cord Stimulation (SCS) would be effective in a patient and how SCS should be targeted. Vertebrae disc 300 including nucleus pulposus 310 and other bony structures such as the lamina 320 covers the dura 340 that surrounds the spinal cord 330 with its spinal nerve roots 350. Ultrasound transducer 370 is pressed against skin 360 and generates ultrasound beam 380 that neuromodulates nerves within spinal cord 330. Bilateral neuromodulation of spinal cord 330 can be performed. For ultrasound to be effectively transmitted to and through the skin and to target spinal-cord target, coupling must be put into place. A layer of ultrasound transmission gel (not shown) is placed between the face of the ultrasound transducer and the skin over the target. If filling of additional space (e.g., within the transducer housing) is necessary, an ultrasound transmission medium (for example Dermasol from California Medical Innovations) can be used. In another embodiment, multiple ultrasound transducers whose beams intersect at that target replace an individual ultrasound transducer for that target. In still another embodiment, mechanical perturbations are applied radially or axially to move the ultrasound transducers. Ultrasound neuromodulation locations that are successful suggest sites at which application of Spinal Cord Stimulation is likely to also be successful. In an embodiment of the invention, effective parameters of the ultrasound neuromodulation can provide insight into the parameters to be used in SCS, for instance pulsing frequency, relative intensity, and whether a stimulus is monophasic or biphasic.

Transducer array assemblies of the type used in ultrasound neuromodulation may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2nd International Symposium on Therapeutic Ultrasound—Seattle—31/07—Feb. 8, 2002), typically with numbers of ultrasound transducers of 300 or more. Keramos-Etalon and Blatek in the U.S. are other custom-transducer suppliers. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

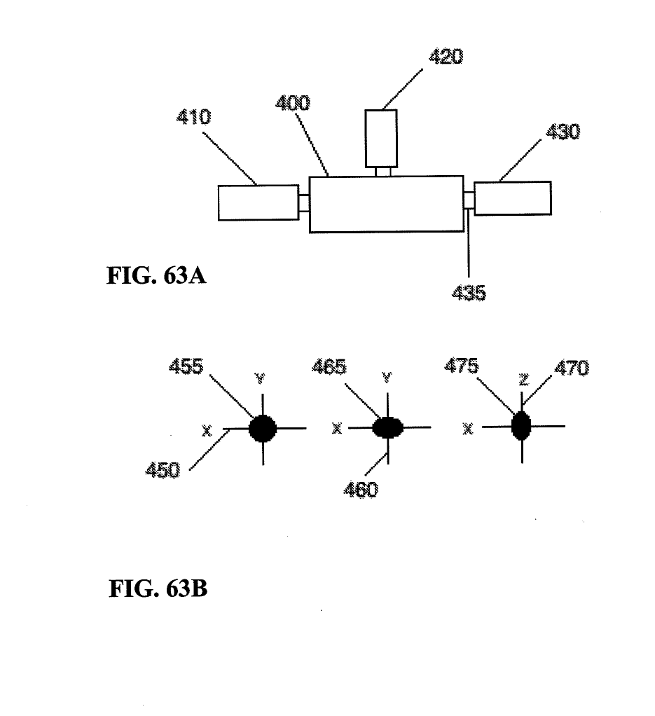

FIGS. 63A and 63B show the mechanism for mechanical perturbation of the ultrasound transducer. In FIG. 63A illustrates a plan view with mechanical actuators 420 and 430 moving ultrasound transducer 400 in and out and left respectively. Actuator rod 435 provides the mechanical interface between mechanical actuator 430 and ultrasound transducer 400 as an example. An equivalent mechanical actuator 410 is shown schematically and moves ultrasound transducer 400 along an axis perpendicular to the page. The combination of actuator 410, actuator 420 and actuator 430 can provide three-dimensional scan patterns under control of the system and under user input as described herein. Such mechanical actuators can have alternative configurations such as motors, vibrators, solenoids, magnetostrictive, electrorestrictive ceramic and shape memory alloys. Piezo-actuators such as those provided by DSM can have very fine motions of 0.1% length change. FIG. 63B shows effects on the focused ultrasound modulation focused at the target. The three axes are axis 450 (x,y), axis 460 (x,y,) and axis 470 (x,z). As demonstrated on the axes 450 the excursions along x and y from actuator 430 and actuator 420, respectively, are equal so the resultant pattern is a circle. As demonstrated on axis 460 the excursion due to actuator 430 is greater than that actuator 420 so the resultant pattern is longer along the x axis than the y axis. As demonstrated on axis 470, the excursion is longer along the z axis than the x axis to the resultant pattern is long along the z axis than the x axis. Not shown is the inclusion of the impacts of actuation along the axis perpendicular to the page, although this will be readily understood by a person of ordinary skill in the art. In each case, the pattern of movement can be determined so as to correspond to the shape of the target site treated with the modulated ultrasound beam.

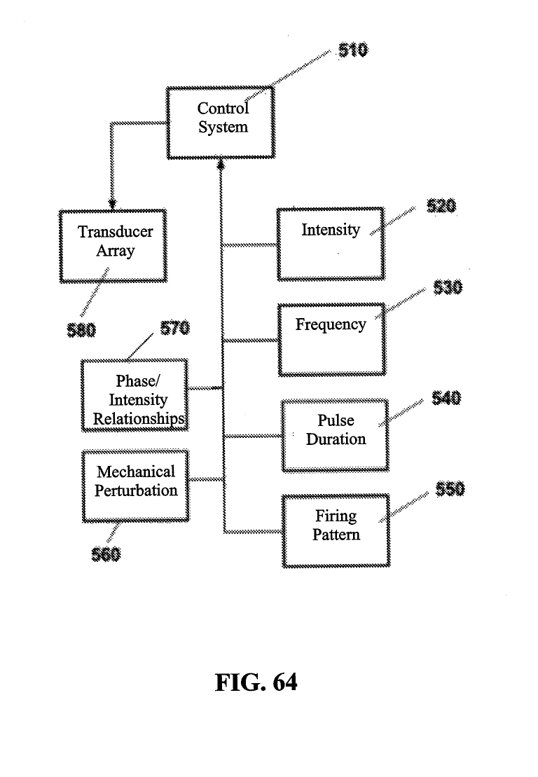

FIG. 64 shows an embodiment of a control circuit. The positioning and emission characteristics of transducer array 580 are controlled by control system 510 with control input with neuromodulation characteristics determined by settings of intensity 520, frequency 530, pulse duration 540, firing pattern 550, mechanical perturbation 560, and phase/intensity relationships 570 for beam steering and focusing on neural targets.

The patient can be treated in one or more of many ways. For example, the patient can be treated with one or more sessions. The pulse can be shaped in many ways with one or more of macro pulse shaping and amplitude modulation, for example. For example, the ultrasound acoustic carrier frequency can be pulse shape modulated, so as to provide shaped stimulation pulses comprising ultrasound having the carrier frequency.

In another embodiment, a feedback mechanism to ultrasound stimulation is applied such as functional Magnetic Resonance Imaging (fMRI), Positive Emission Tomography (PET) imaging, video-electroencephalogram (V-EEG), acoustic monitoring, thermal monitoring, and patient feedback. In an embodiment, feedback is provided by a measurement specific to a symptom or disease state of a patient.

In still other embodiments, other energy sources are used in combination with or substituted for ultrasound transducers such as Transcranial Magnetic Stimulation (TMS) or transcranial Direct Current Stimulation (tDCS). Therapies that can be preplanned with ultrasound neuromodulation are usually invasive modalities such as Deep-Brain Stimulation (DBS), optogenetics application, or stereotactic radiosurgery. Alternatively ultrasound neuromodulation can be used for preplanning for non-invasive neuromodulation such as Transcranial Magnetic Stimulation (TMS) or transcranial Direct Current Stimulation (tDCS). In either or both cases preplanning can be done for one or multiple modalities including the aforementioned and other therapies such as behavioral therapies and drugs.

The operator can set the variables for preplanning or diagnostic ultrasound neuromodulation or the patient can do so in a self-actuated manner. In some self-actuated embodiments, the patient can expedite the process due to their ability to tune the ultrasound neuromodulation to obtain its best results through subjective assessments of whether a symptom or disease state is mitigated with a particular ultrasound session.

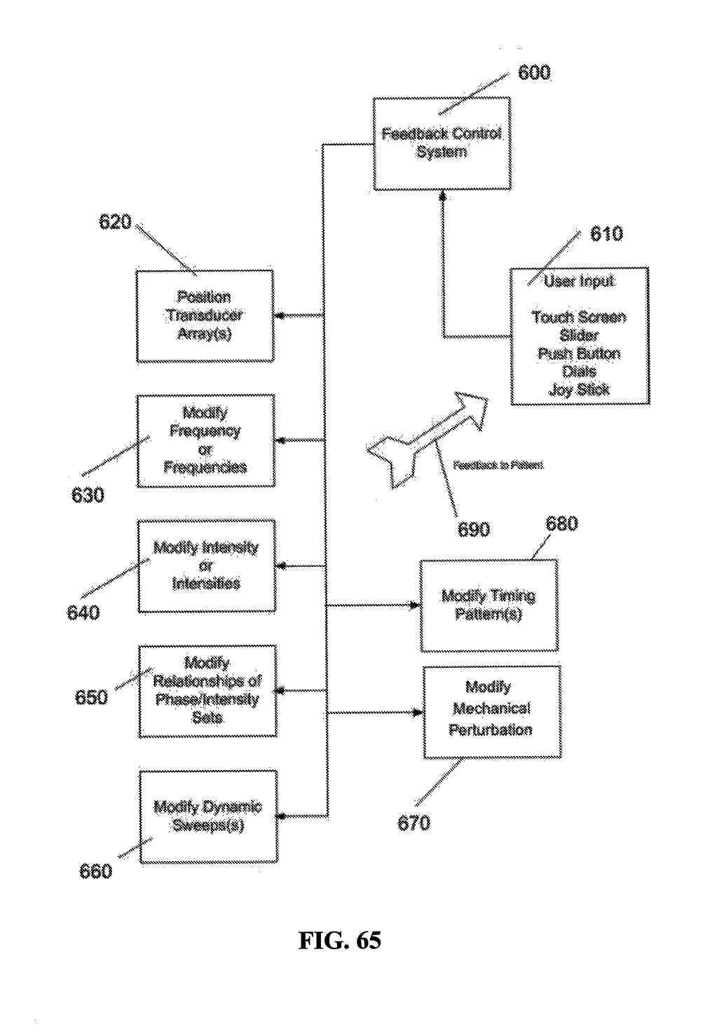

FIG. 65 shows the basic feedback circuit. Feedback Control System 600 receives its input from User Input 610 and provides control output for positioning ultrasound transducer arrays 620, modifying pulse frequency or frequencies 630, modifying intensity or intensities 640, modifying relationships of phase/intensity sets 650 for focusing including spot positioning via beam steering, modifying dynamic sweep patterns 660, modifying mechanical perturbation 670, and/or modifying timing patterns 680. Feedback to the patient 690 occurs based on a measured physiological cognitive, subjective, or other disease- or health-related measurement (for example increase or decrease in pain or decrease or increase on tremor). User Input 520 can be provided via a touch screen, slider, dials, joystick, or other suitable means. Often the user can be the best judge concerning which neuromodulation parameters are most effective, either changing one variable of ultrasound at a time or multiple ultrasound waveform variables. Examples of the application of patient feedback are the patient adjusting neuromodulation parameters to ameliorate pain, depression, and resting tremor. Another is a patient with a transected spinal cord directly turning on the neuromodulation to empty a neurogenic bladder.

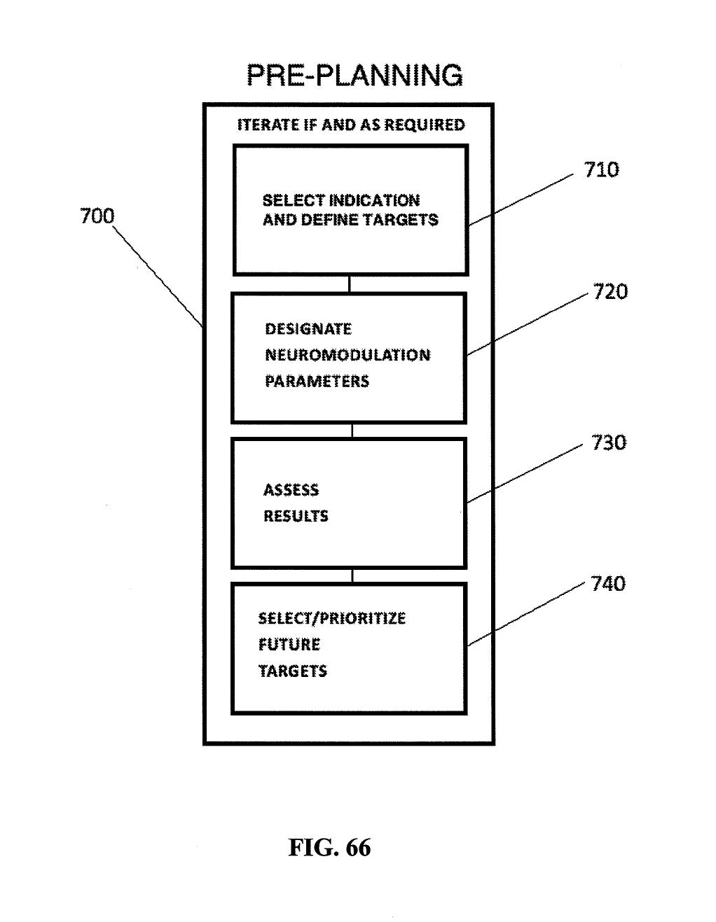

FIG. 66 shows a method 700 of pre-planning for neuromodulation therapy. The neuromodulation therapy may comprise one or more of Ultrasound Neuromodulation, Transcranial Magnetic Stimulation (TMS) or Deep Brain Stimulation (DBS)) or ablative therapy, for example. Each of the steps within method 700 may be performed iteratively, for example. A step 710 comprises selecting an indication for treatment and defining related targets sites. The indication may comprise one or more indications as described herein such as one or more of Parkinson's Disease, Depression/Bipolar Disorder, or Spinal Cord Pain, for example. A step 720 comprises designating ultrasound neuromodulation parameters to apply in either one or multiple neuromodulation sessions, for example. The neuromodulation parameters may comprise one or more known parameters and can be determined by one of ordinary skill in the art based on the embodiments described herein. A step 730 comprises assessing the results in response to the ultrasound neuromodulation in order to determine stimulation effect, if present. The presence of a stimulation effect can confirm the site as suitable for use with treatment. A step 740 comprises one or more of selecting or prioritizing targets for future treatment based on the assessment of the results, such that the sites are confirmed prior to treatment.

Table 1 shows a table suitable for incorporation with pre-planning in accordance with embodiments as described herein.

TABLE 1

| Condition-Input | Target Site Evaluated | Assessment | Subsequent Treatment |
|---|---|---|---|
| Depression | Cingulate Genu | Depression/Normal | DBS targeted to |
| | | | cingulate genu |
| Parkinson's | DBS, STN, GPi | Tremor | levodopa, dopamine |
| | | | agonists, MAO-B |
| | | | inhibitors, and other |
| | | | drugs such as |
| | | | amantadine and |
| | | | anticholinergics |
| Essential Tremor | (Vim) | Tremor | beta blockers, |
| | | | propranolol, |
| | | | antiepileptic agents, |
| | | | primidone, or |
| | | | gabapentin |
| Bipolar Disorder | Nucleus accumbens, the | Structured Clinical | DBS, lithium, valproic |
| | subcallosal cingulate | Interview for DSM-IV | acid, divalproex, |
| | (Area 25) | (SCID), the Schedule for | lamotrigine, quetiapine, |
| | | Affective Disorders and | antidepressants, |
| | | Schizophrenia (SADS), | Symbyax, clonazepam, |
| | | or other bipolar | lorazepam, diazepam, |
| | | assessment tool | chlordiazepoxide, and |
| | | | alprazolam |
| Spinal Cord Pain | Various levels of the | Comparative pain scale | Level of the spinal |
| | spinal column; white | or galvanic skin | column and site for |
| | matter and ganglia | response | electrical stimulation, |
| | | | ultrasound |
| | | | neuromodulation, or |
| | | | surgical intervention |

With regards to the Nucleus accumbens, supportive data can be found be one of ordinary skill in the art on the world wide web (www.clinicaltrials.gov/ct2/show/NCT01372722). With regards to the subcallosal cingulate (Area 25), supportive data can be found be one of ordinary skill in the art on the world wide web (www.dana.org/media/detail.aspx?id=35782). With regards to the Schedule of Affective Disorders and Schizophrenia, supportive data can be found by one of ordinary skill in the art at on the world wide web (www.ncbi.nlm.nih.gov/pmc/articles/PMC2847794/). With regards to treatment and drugs related to bipolar disorder, supportive data can be found on the world wide web by one of ordinary skill in the art (http://www.mayoclinic.com/health/bipolar-disorder/DS00356/DSECTION=treatments-and-drugs).

The method 700 can be used to confirm treatment of the patient based on the patient's response to target site evaluated. For the condition input and target site evaluated, a subsequent treatment can be selected that acts on the target site evaluated, for example as described herein with reference to Table 1.

Although the above steps show method 700 of planning a treatment of a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

One or more of the steps of the method 700 may be performed with the circuitry as described herein, for example one or more of the processor or logic circuitry such as programmable array logic for field programmable gate array. The circuitry may be programmed to provide one or more of the steps of method 700, and the program may comprise program instructions stored on a computer readable memory or programmed steps of the logic circuitry such as the programmable array logic or the field programmable gate array, for example.

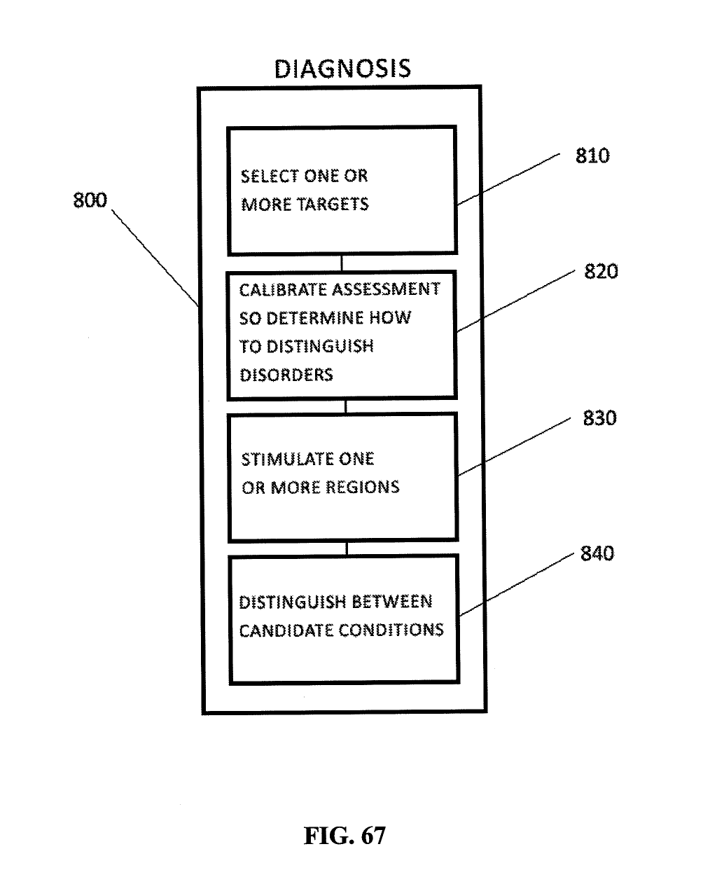

FIG. 67 shows a method 800 of diagnosis of a patient. A step 810 comprises selection of one or more target sites as described herein. A step 820 comprises calibrating an assessment to determine how to distinguish candidate disorders based on elicited effects consistent with one disorder versus another disorder, for example. A step 830 comprises stimulating the one or more target sites with ultrasound as described herein. A step 840 comprises distinguishing among a plurality of candidate conditions. The process 800 provides information for guiding treatment irrespective of the treatment. The treatment may comprise one or more treatments as described herein such as neuromodulation, surgery, or medication, for example. Assessments can be made by direct observation or by instruments such as the known Visual Analog Scale for pain (H. Breivik, H., Borchgrevink, P. C., Allen, S. M., Rosseland, L. A., Romundstad, L., Breivik Hals, E. K., Kvarstein, G., and A. Stubhaug, “Assessment of Pain,” Br J. Anaesth. 2008; 101(1):17-24.) or motor skill assessments for Parkinson's disease (Motor Bruininks-Oseretsky Test of Motor Proficiency, Second Edition (BOT-2), Authors: Robert H. Bruininks, PhD & Brett D. Bruininks, (for ages for four through 21) and Bruininks Motor Ability Test (BMAT), Authors: Brett D. Bruininks & Robert H. Bruininks, PhD (for adults), both by Pearson Education, Inc.).

Table 2 shows a table suitable for incorporation with diagnosis in accordance with embodiments as described herein.

TABLE 2

| | Target Site(s) | | |
|---|---|---|---|
| Symptom-Input | Evaluated-Input | Assessment/Indicator | Condition-Output |
| Depression/Normal | Cingulate Genu | Depression/Normal | Depression |
| Tremor | DBS, STN, or GPi | Tremor | Parkinson's |
| Tremor | Vim | Tremor | Essential Tremor |
| Bipolar behavior | Nucleus accumbens, the | Structured Clinical | Bipolar Disorder |
| | subcallosal cingulate | Interview for DSM-IV | |
| | (Area 25) | (SCID), the Schedule for | |
| | | Affective Disorders and | |
| | | Schizophrenia (SADS), | |
| | | or other bipolar | |
| | | assessment tool | |
| Pain | Spinal Cord; Various | Comparative pain scale | Spinal Cord Pain |
| | levels of the spinal | or galvanic skin | |
| | column; white matter | response | |
| | and ganglia | | |

Although the above steps show method 800 of diagnosing a patient in accordance with embodiments, a person of ordinary skill in the art will recognize many variations based on the teaching described herein. The steps may be completed in a different order. Steps may be added or deleted. Some of the steps may comprise sub-steps. Many of the steps may be repeated as often as if beneficial to the treatment.

One or more of the steps of the method 800 may be performed with the circuitry as described herein, for example one or more of the processor or logic circuitry such as programmable array logic for field programmable gate array. The circuitry may be programmed to provide one or more of the steps of method 800, and the program may comprise program instructions stored on a computer readable memory or programmed steps of the logic circuitry such as the programmable array logic or the field programmable gate array, for example.

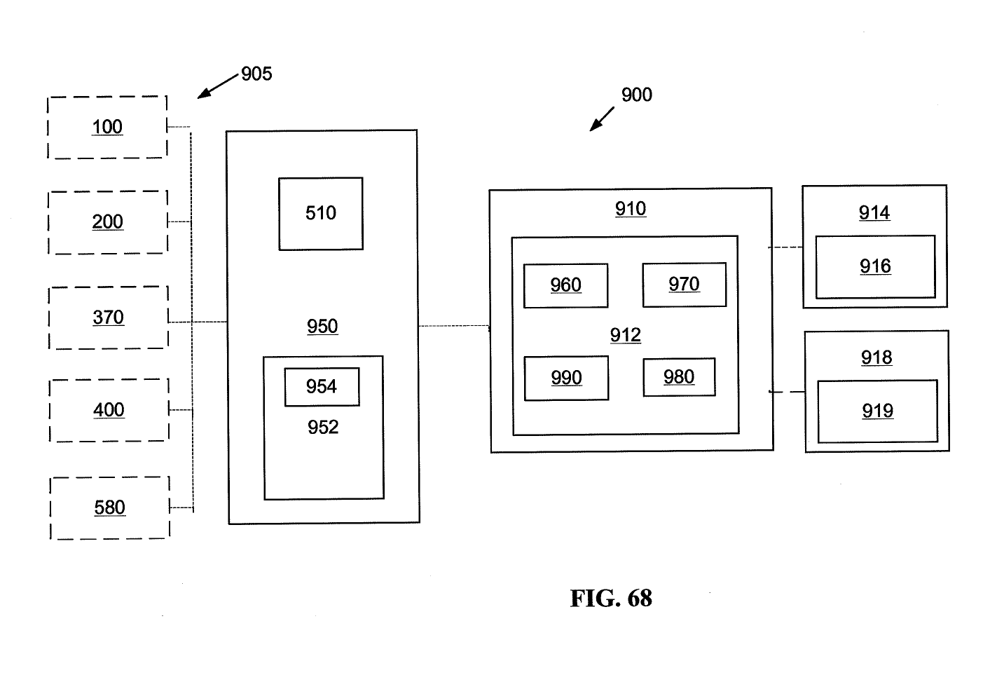

FIG. 68 shows an apparatus 900 for one or more of preplanning or diagnosing the patient, in accordance with embodiments. The apparatus 900 comprises an ultrasound source 905. The ultrasound source 905 comprises a source of ultrasound as described herein. The ultrasound source 905 may comprise a head 100, a head 200, a transducer 370, a transducer 400, or a transducer array 580 as described herein for example.

The apparatus 900 comprises a controller 950 coupled to the ultrasound source 905. The controller 950 comprises a processor 952 having a computer readable medium 954. The computer readable memory 954 may comprise instructions for controlling the ultrasound source. The controller 950 may comprise one or more components of the control system 510 as described herein.

The apparatus 900 comprises a processor system 910. The processor system 910 is coupled with a control system. The processor 910 comprises a computer readable memory 912 having instructions of one or more computer programs embodied thereon. The computer readable memory 912 comprises instructions 960. The instructions 960 comprise one or more instructions of the feedback control system 600 and corresponding methods as described herein. The computer readable memory 912 comprises instructions 970. The instructions 970 comprise one or more instructions to implement one or more steps of the preplanning method 700 as described herein. The computer readable memory 980 comprises instructions to implement one or more steps of the method 980 of diagnosing a patient as described herein. The computer readable memory 912 comprises instructions 990 to coordinate the components as described herein and the methods as described herein. For example, the instructions 990 may comprise a user responsive switch to select preplanning method 970 or instructions to diagnose the patient 980 based on user preference. The computer readable memory may comprise information of one or more of Table 1 or Table 2 so as to plan treatment of the patient and diagnose the patient, in accordance with embodiments as described herein.

The processor system 910 is coupled to a user interface 914. The user interface 914 may comprise a display 916 such as a touch screen display. The user interface 914 may comprise a handheld device such as a commercially available iPhone, Android operating system device, such as, a Samsung Galaxy S3 or other known handheld device such as an iPad, tablet computer, or the like. The user interface 914 can be coupled with a processor system 910 with communication methods and circuitry. The communication may comprise one or more of many known communication techniques such as WiFi, Bluetooth, cellular data connection, and the like. The processor system 910 is configured to communicate with a measurement apparatus 918. The measurement apparatus 918 comprises patient measurement data storage 919 that can be stored on a computer readable memory. The processor system 910 is in communication with the measurement apparatus 918 with communication that may comprise known communication as described herein. The processor system 910 is configured to communicate with the controller 950 to transmit the signals for use with the ultrasound source 905 in for implementation with one or more components of control system 510 as described herein.

The apparatus 900 allows ultrasound stimulation adjustments in variables such as carrier frequency and/or neuromodulation frequency, pulse duration, pulse pattern, mechanical perturbation, as well as the direction of the energy emission, intensity, frequency, phase/intensity relationships to targeting and accomplishing up-regulation and/or down-regulation, dynamic sweeps, and position. The user can input these parameters with the user interface, for example.

Reference is made to the following publications, which are provided herein to clearly and further show that the embodiments of the methods and apparatus as described herein are clearly enabled and can be practiced by a person of ordinary skill in the art without undue experimentation.

Clinical stimulation of the Cingulate Genu in humans is described by Mayberg et al. (Mayberg, Helen S., Lozano, A.M., Voon, Valerie, McNeely, Heather E., Seminowicz, D., Hamani, C., Schwalb, J. M., and S. H., Kennedy, “Deep Brain Stimulation for Treatment-Resistant Depression,” Neuron, Volume 45, Issue 5, 3 Mar. 2005, Pages 651-660), for example.

Patient response to Stimulation of the Subthalamic Nucleus and Globus Pallidus interna can produce measurable patient results suitable for one or more of diagnosis or confirmation as described herein. (Anderson et al. (Anderson, V C, Burchiel, K J, Hogarth, P, Favre, J, and J P Hammerstad, “Pallidal vs subthalamic nucleus deep brain stimulation in Parkinson disease,” Arch Neurol. 2005 April; 62(4):554-60)

The stimulation of deep-brain structures with ultrasound has been suggested previously (Gavrilov L R, Tsirulnikov E M, and I A Davies, “Application of focused ultrasound for the stimulation of neural structures,” Ultrasound Med Biol. 1996; 22(2):179-92. and S. J. Norton, “Can ultrasound be used to stimulate nerve tissue?,” BioMedical Engineering OnLine 2003, 2:6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton describes a strong DC magnetic field as well and describes the mechanism as that given that the tissue to be stimulated is conductive that particle motion induced by an ultrasonic wave will induce an electric current density generated by Lorentz forces, such that ultrasound is suitable for combination with TMS in accordance with embodiments as described herein.

A person of ordinary skill in the art can combine ultrasound with TMS in accordance with the embodiments as described herein.

Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to affect a multi-beam output, suitable for combination in accordance with embodiments as described herein. Transducers may coordinated by a computer and used in conjunction with an imaging system, preferable an fMRI (functional Magnetic Resonance Imaging), but possibly a PET (Positron Emission Tomography) or V-EEG (Video-Electroencephalography) device. The user may interact with the computer to direct the FUP to the desired point in the brain, sees where the stimulation actually occurred by viewing the imaging result, and thus adjusts the position of the FUP accordingly.

### Part XIII: Planning and Using Sessions of Ultrasound for Neuromodulation

In some variations, the purpose of the inventions described herein is to provide methods and systems and methods for neuromodulation of deep-brain targets using ultrasound delivered in sessions. Examples of session types include periodic sessions over extended time typically means a single session of length on the order of 15 to 60 minutes repeated daily or five days per week over one to six weeks. Other lengths of session or number of weeks of neuromodulation are applicable, such as session lengths up to 2.5 hours and number of weeks ranging from one to eight. Period sessions over compressed time typically means a single session of length on the order of 30 to 60 minutes repeated during awake hours with inter-session times of 15 minutes to 60 minutes over one to three days. Other inter-session times such as 15 minutes to three hours and days of compressed therapy such as one to five days are applicable. Maintenance consists of periodic sessions at fixed intervals or on as-needed maintenance tune-ups. Maintenance categories are maintenance post-completion of original treatment at fixed intervals and maintenance post-completion of original treatment with as-needed maintenance tune-ups. An example of the former are with one or more 50-minutes sessions during week 2 of months four and eight, and of the latter is one or more 50-minute sessions during week 7 because a tune up is needed at that time as indicated by return of symptoms. Use of sessions is important for the retraining of neural pathways for change of function, maintenance of function, or restoration of function. Retraining over time, with its ongoing reinforcement, can allow more effectively achievement of desired impacts. Another consideration is the desirability for practical reasons to limit tying up the time of the patient depending on the individual situation. Such neuromodulation systems can produce applicable acute or long-term effects. The latter occur through Long-Term Depression (LTD) or Long-Term Potentiation (LTP) via training. Included is control of direction of the energy emission, intensity, frequency (carrier frequency and/or neuromodulation frequency), pulse duration, pulse pattern, and phase/intensity relationships to targeting and accomplishing up-regulation and/or down-regulation.

The stimulation frequency for inhibition is lower than 400 Hz (depending on condition and patient). The stimulation frequency for excitation is in the range of 600 Hz to 4.5 MHz. In this invention, the ultrasound acoustic frequency is in range of 0.25 MHz to 0.85 MHz with power generally applied less than 65 mW/cm2 but also at higher target- or patient-specific levels at which no tissue damage is caused. The acoustic frequency is modulated at the lower rate to impact the neuronal structures as desired (e.g., say typically 400 Hz for inhibition (down-regulation) or 600 Hz for excitation (up-regulation). The modulation frequency (superimposed on the carrier frequency of say 0.55 MHz or similar) may be divided into pulses 0.1 to 20 msec. repeated at frequencies of 2 Hz or lower for down regulation and higher than 2 Hz for up regulation although this will be both patient and condition specific. The focus area of the pulsed ultrasound js 0.1 to 1 inch in diameter. The number of ultrasound is between 1 and 100. Ultrasound therapy can be combined with therapy using other devices (e.g., Transcranial Magnetic Stimulation (TMS)).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. Keramos-Etalon can supply a 1-inch diameter ultrasound transducer and a focal length of 2 inches that with 0.4 Mhz excitation will deliver a focused spot with a diameter (6 dB) of 0.29 inches. Typically, the spot size will be in the range of 0.1 inch to 0.6 inch depending on the specific indication and patient. A larger spot can be obtained with a 1-inch diameter ultrasound transducer with a focal length of 3.5″ which at 0.4 MHz excitation will deliver a focused spot with a diameter (6 dB) of 0.51.″ Even though the target is relatively superficial, the transducer can be moved back in the holder to allow a longer focal length. Other embodiments are applicable as well, including different transducer diameters, different frequencies, and different focal lengths. Other ultrasound transducer manufacturers are Blatek and Imasonic. In an alternative embodiment, focus can be deemphasized or eliminated with a smaller ultrasound transducer diameter with a shorter longitudinal dimension, if desired, as well. Ultrasound conduction medium will be required to fill the space.

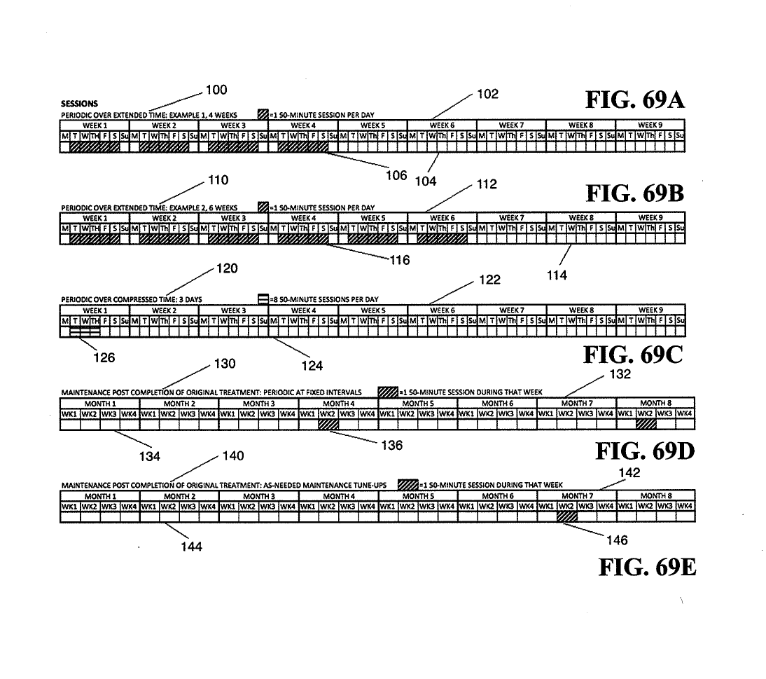

FIGS. 69A-69E shows a diagram of exemplar session types for both initial treatment and maintenance sessions. FIG. 69A illustrates example 100, Periodic Over Extended Time with 4 weeks of treatment where time divisions are weeks 102 divided into days 104 with 50-minute sessions on indicated days 106. For all of these examples, the session length could be longer or shorter than 50 minutes. FIG. 69B illustrates example 110, Periodic Over Extended Time with 6 weeks of treatment where time divisions are weeks 112 divided into days 114 with 50-minute sessions on indicated days 116. FIG. 69C illustrates example 120, Periodic Over Compressed Time with 3 days of treatment where time divisions are weeks 122 divided into days 124 with 50-minute sessions on indicated days 166. FIG. 69D illustrates example 130, Maintenance Post Completion of Original Treatment at Fixed Intervals where time divisions are months 132 divided into weeks 134 with 50-minute sessions during indicated weeks 136. FIG. 69E illustrates example 140, Maintenance Post Completion of Original Treatment with As-Needed Maintenance Tune-Ups where time divisions are months 142 divided into weeks 144 with 50-minute sessions during indicated week 146.

An example of one of the treatment to which sessions would be applicable is depression and bipolar disorder. Multiple targets can be neuromodulated singly or in groups to treat depression or bipolar depression. To accomplish the treatment, in some cases the neural targets will be up regulated and in some cases down regulated, depending on the given neural target. Targets have been identified by such methods as PET imaging, fMRI imaging, and clinical response to Transcranial Magnetic Stimulation (TMS). The Left Prefrontal Cortex would be up regulated (George, M. S., Wassermann, E. M., Williams, W. A., Callahan A., Ketter, T. A., Basser, P., Hallett, M., and R. M. Post, “Daily repetitive transcranial magnetic stimulation (rTMS) improves mood in depression,” Neuroreport 1995; 6:1853-1856), the Right Prefrontal Cortex down regulated (Menkes, D. L., Bodnar, P., Ballesteros, R. A., and M. R. Swenson, “Right frontal lobe slow frequency repetitive transcranial magnetic stimulation (SF r-TMS) is an effective treatment for depression: a case-control pilot study of safety and efficacy,” J Neurol Neurosurg Psychiatry 1999; 67:113-115), Orbito-Frontal Cortex (OFC) (Lee, Seong, et al., 2007 (Lee, B. T., Seong, Whi Cho, Hyung, Soo Khang, Lee. B. C., Choi I. G., Lyoo, I. K., and B. J. Ham, “The neural substrates of affective processing toward positive and negative affective pictures in patients with major depressive disorder,” Prog Neuropsychopharmacol Biol Psychiatry. 2007 Oct. 1; 31(7):1487-92. Epub 2007 Jul. 5)) would be up regulated, the Anterior Cingulate Cortex (ACC) would be up regulated (Lee, Seong, et al., 2007), the Subgenu Cingulate (Johansen-Berg, H., Gutman, D. A., Behrens, T. E., Matthews, P. M., Rushworth, M. F., Katz, E., Lozano, A. M., and H. S. Mayberg, “Anatomical connectivity of the subgenual cingulate region targeted with deep brain stimulation for treatment-resistant depression,” Cereb Cortex. 2008 June; 18(6):1374-83. Epub 2007 Oct. 10.) down regulated, the Right Insula (Lee, Seong, et al., 2007) up regulated, the left Insula (Lee, Seong, et al., 2007) down regulated, the Nucleus Accumbens (Hauptman, J. S., DeSalles, A. A., Espinoza, R., Sedrak, M., and W. Ishida, “Potential surgical targets for deep brain stimulation in treatment-resistant depression.,” Neurosurg Focus. 2008; 25(1):E3) up regulated, the Caudate Nucleus (Lee, Seok et al, 2008 (Lee, B. T., Seok, J. H., Lee, B. C., Cho, S. W., Yoon, B. J., Lee, K. U., Chae, J. H., Choi, I. G., and B. J. Ham, “Neural correlates of affective processing in response to sad and angry facial stimuli in patients with major depressive disorder, “Prog Neuropsychopharmacol Biol Psychiatry. 2008 Apr. 1; 32(3):778-85. Epub 2007 Dec. 23.)) up regulated, the Amygdala (Lee, Seong, et al., 2007) down regulated, and the Hippocampus (Lee, Seok et al, 2008) up regulated. The specific targets and/or whether the given target is up regulated or down regulated, can depend on the individual patient and relationships of up regulation and down regulation among targets, and the patterns of stimulation applied to the targets. In some cases neuromodulation will be bilateral and in others unilateral.

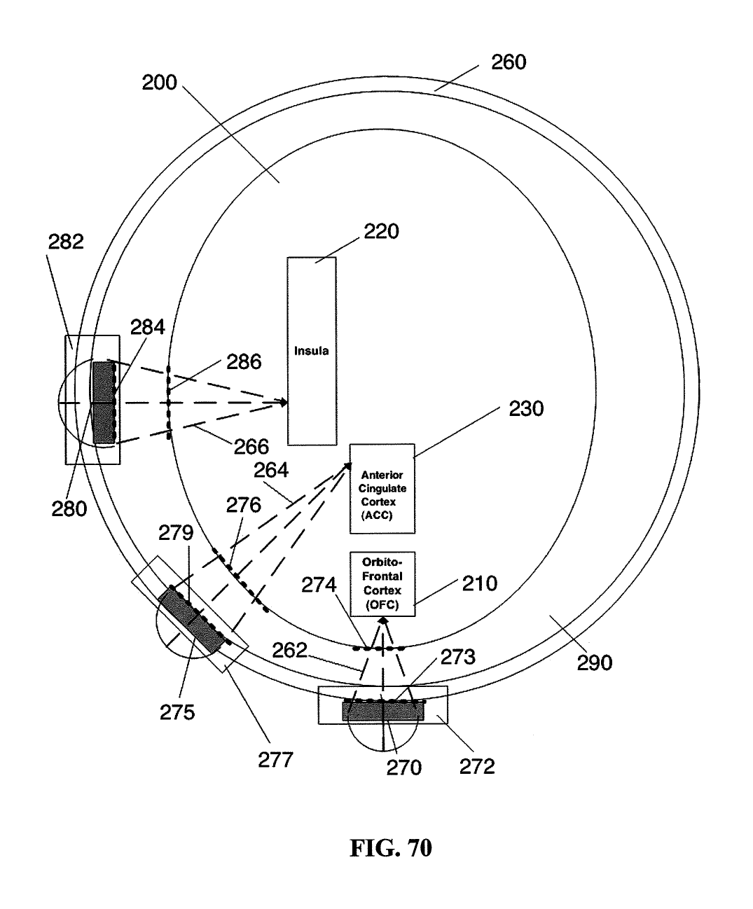

FIG. 70 shows a set of ultrasound transducers targeting to treat depression and bipolar disorder. The head 200 contains the three targets, Orbito-Frontal Cortex (OFC) 210, Insula 220, and Anterior Cingulate Cortex (ACC) 130. These targets are hit by ultrasound from transducers 270 with ultrasound beam 262, 275 with ultrasound beam 264, and 280 with ultrasound beam 266, with their respective holders 272, 277, and 282 fixed to track 260. Ultrasound transducer 270 is shown targeting the OFC 210, transducer 275 is shown targeting the ACC 230, and transducer 280 is shown targeting the Insula 220. Transducer 270 is moved radially in or out of holder 272 and fixed into position. In like manner, transducer 275 is moved radially in or out of holder 277 and fixed into position and transducer 280 is moved radially in or out of holder 282 and fixed into position. In other embodiments, transducers 270, 275, and 280 are directly fixed on track 260. For ultrasound to be effectively transmitted to and through the skull and to brain targets, coupling must be put into place. Ultrasound transmission (for example Dermasol from California Medical Innovations) medium 290 is interposed with one mechanical interface to the ultrasound transducers 270, 275, 280 (completed by a layers of ultrasound transmission gel 273, 279, 284) and the other mechanical interface to the head 100 (completed by a layers of ultrasound transmission gel 274, 276, 286). This figure shows a fixed configuration where the appropriate radial (in-out) positions have determined through patient-specific imaging (e.g., PET or fMRI) and the holders positioning the ultrasound transducers are fixed in the determined positions. To support this embodiment, treatment-planning software is used taking the image-determined target positions and output instructions for manual or computer-aided manufacture of the holders. Alternatively positioning instructions can be output for the operator to position the blocks holding the transducers to be correctly placed relative to the support track. In one embodiment, the transducers positioned using this methodology can be aimed up or down and/or left or right for correct flexible targeting.

Transducer array assemblies of this type may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2nd International Symposium on Therapeutic Ultrasound—Seattle—31/07—Feb. 8, 2002), typically with numbers of ultrasound transducers of 300 or more. Keramos-Etalon in the U.S. is another custom-transducer supplier. The power applied will determine whether the ultrasound is high intensity or low intensity (or medium intensity) and because the ultrasound transducers are custom, any mechanical or electrical changes can be made, if and as required. At least one configuration available from Imasonic (the HIFU linear phased array transducer) has a center hole for the positioning of an imaging probe. Keramos-Etalon also supplies such configurations.

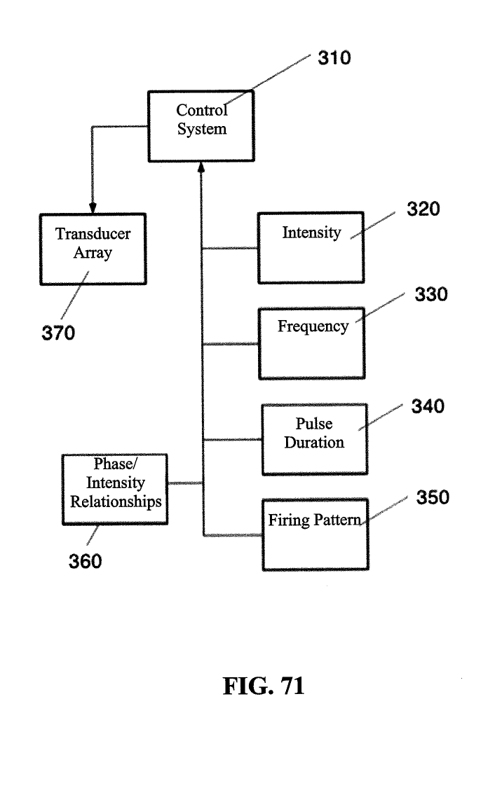

FIG. 71 shows an embodiment of a control circuit. The positioning and emission characteristics of transducer array 370 are controlled by control system 310 with control input with neuromodulation characteristics determined by settings of intensity 320, frequency 330, pulse duration 340, firing pattern 350, and phase/intensity relationships 360 for beam steering and focusing on neural targets.

In another embodiment, a feedback mechanism is applied such as functional Magnetic Resonance Imaging (fMRI), Positive Emission Tomography (PET) imaging, video-electroencephalogram (V-EEG), acoustic monitoring, thermal monitoring, and patient feedback.

In still other embodiments, other energy sources are used in combination with or substituted for ultrasound transducers that are selected from the group consisting of Transcranial Magnetic Stimulation (TMS), deep-brain stimulation (DBS), optogenetics application, radiosurgery, Radio-Frequency (RF) therapy, behavioral therapy, and medications.

The invention allows stimulation adjustments in variables such as, but not limited to, direction of the energy emission, intensity, frequency (carrier frequency and/or neuromodulation frequency), pulse duration, pulse pattern, and phase/intensity relationships to targeting and accomplishing up-regulation and/or down-regulation, dynamic sweeps, and position.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In general, when a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of neuromodulating a patient by applying stimulation.

2. The method of claim 1, wherein applying stimulation comprises neuromodulating one or a plurality of deep-brain targets, the method further comprising using multiple therapeutic modalities, the method further comprising:
applying a plurality of therapeutic modalities to a deep-brain target;
applying power to each of the on-line therapeutic modalities via a control circuit thereby neuromodulating the activity of the deep brain target regions, and
working in coordination with the off-line therapeutic modalities.

3. The method of claim 1, wherein applying stimulation comprises neuromodulating one or a plurality of deep-brain targets by ultrasound stimulation, the method further comprising:
aiming one or a plurality of ultrasound transducers at one or a plurality of deep-brain targets;
applying power to each of the ultrasound transducers via a control circuit thereby neuromodulating the activity of the deep brain target region;
moving one or a plurality of transducers around a track surrounding the mammal's head.

4. The method of claim 1, wherein applying stimulation comprises neuromodulating one or a plurality of deep-brain targets by ultrasound stimulation, the method further comprising:
using a mechanism for aiming one or a plurality of ultrasound transducers at one or a plurality of deep-brain targets;
applying power to each of the ultrasound transducers via a control circuit thereby modulating the activity of the deep brain target region;
providing a mechanism for feedback from the patient based on the acute sensory or motor conditions of the patient; and
using that feedback to control one or more parameters to maximize the desired effect.

5. The method of claim 1, wherein applying stimulation comprises neuromodulating one or a plurality of deep-brain targets by non-invasively stimulating neural structures such as the brain using ultrasound stimulation, the method further comprising:
aiming an ultrasound transducer at the selected neural target;
macro-shaping the pulse outline of the tone burst; and
applying pulsed power to said ultrasound transducer via a control circuit thereby whereby the neural structure is neuromodulated.

6. The method of claim 1, wherein applying stimulation comprises neuromodulating one or a plurality of deep-brain targets by ultrasound neuromodulation, the method further comprising:
providing one or a plurality of ultrasound transducers;
aiming the beams of said ultrasound transducers at one or a plurality of applicable neural targets; and
modulating the ultrasound transducers with patterned stimulation, whereby the one or a plurality of neural targets are each neuromodulated producing regulation selected from the group consisting of up-regulation and down-regulation.

7. The method of claim 1, wherein applying stimulation comprises neuromodulating one or a plurality of deep-brain targets wherein stimulation comprises ultrasound neuromodulation of one or a plurality of deep-brain targets, the method further comprising:
attaching a plurality of ultrasound transducers to a positioning frame; and
aiming the beams from the ultrasound transducers so said beams intersect at the one or plurality of targets, whereby the combination of said ultrasound beams neuromodulates the targeted neural structures producing one or a plurality of regulations selected from the group consisting of up-regulation and down-regulation.

8. The method of claim 1, wherein applying stimulation comprises non-invasively neuromodulating the brain using ultrasound stimulation, the method comprising:
aiming an ultrasound transducer at superficial cortex;
applying pulsed power to said ultrasound transducer via a control circuit thereby neuromodulating the target, whereby results are selected from the group consisting of functional and diagnostic.

9. The method of claim 1, wherein applying stimulation comprises:
providing pulsed ultrasound to one or more neural targets of a neural disorder; and
identifying the neural disorder or planning for treatment of the neural disorder based on a response of the one or more neural targets to the pulsed ultrasound.

10. The method of claim 1, wherein neuromodulating a patient by applying stimulation is performed to alleviate a disease condition, the method further comprising:
aiming at least one ultrasound transducer at a target region of a patient's spinal cord, and
applying pulsed power to the transducer to deliver pulsed ultrasound energy to the target region.

11. An ultrasound transducer for neuromodulation of a deep-brain target comprising:
an ultrasound-generation array with a curvature matched to the depth of the target, and
a shape matched to the shape of the target, whereby said ultrasound transducer neuromodulates the targeted neural structures producing regulation selected from the group consisting of up-regulation and down-regulation.

12. A method for treatment planning for neuromodulation of deep-brain targets using ultrasound neuromodulation, the method comprising:
setting up sets of applications and supported transducer configurations with associated capabilities;
executing treatment-planning sessions;
setting parameters for: the session, system recommendations and user acceptance of changes to applications, targets, up- or down-regulation, stimulation frequencies;
iterating through the sets of applications;
iterating through set of targets;
iterating through and applying in designated order one or more variables selected from the group consisting of position, intensity, firing-timing pattern, phase/intensity relationships, dynamic sweeps; and
presenting treatment plan to user who accepts or changes; whereby the treatment to be delivered is tailored to the patient.

13. A method for altering a permeability of a blood-brain barrier in a patient, the method comprising:
aiming at least one ultrasound transducer at least one target in a brain or a spinal cord of a human or animal; and
energizing at least one transducer to deliver pulsed ultrasound energy to the at least one target, wherein permeability of the blood-brain barrier in the vicinity of the target is altered.

14. A method of deep-brain neuromodulation using ultrasound stimulation, the method comprising:
aiming one or a plurality of ultrasound transducer at one or a plurality of neural targets related to the condition being treated, and
applying pulsed power to the ultrasound transducer via a control circuit,
whereby the ultrasound neuromodulation is delivered in sessions.
